(19) United States
(12) Patent Application Publication
(10) Pub. No.: US 2017/0151355 A1
(43) Pub. Date: Jun. 1, 2017

(54) TARGETED RADIOLABELED COMPOUNDS AND THEIR USE FOR THE TREATMENT AND DIAGNOSIS OF CANCER

(71) Applicant: Board of Regents of The University of Nebraska, Lincoln, NE (US)

(72) Inventors: Janina Baranowska-Kortylewicz, Omaha, NE (US); Zbigniew P. Kortylewicz, Omaha, NE (US)

(21) Appl. No.: 15/431,402

(22) Filed: Feb. 13, 2017

Related U.S. Application Data
(62) Division of application No. 13/512,966, filed on Jul. 9, 2012, now abandoned.
(60) Provisional application No. 61/324,342, filed on Apr. 15, 2010.
(60) Provisional application No. 61/284,737, filed on Dec. 23, 2009.

(51) Int. Cl. A61K 51/04; A61K 49/10; A61K 31/7072

(52) U.S. Cl. CPC A61K 51/0491; A61K 31/7072; A61K 49/10; A61K 51/0493

(57) ABSTRACT

Method of using androgen receptor and/or butyrylcholinesterase targeted radiolabeled compounds, e.g., cycloSalingenyl pyrimidine nucleoside monophosphates, for the treatment and diagnosis of cancer.

### CROSS-REFERENCE TO RELATED APPLICATIONS

The present application claims the benefit of U.S. Provisional Patent Applications Nos. 61/324,342, filed Apr. 15, 2010 and 61/248,737, filed Dec. 23, 2009, the entire disclosures of which are incorporated by reference herein.

Pursuant to 35 U.S.C. Section 202(c), it is acknowledged that the United States Government has certain rights in the invention described herein, which was made in part with funds from the Department of Defense, Grant No. W81XWH-04-1-0463.

### BACKGROUND OF THE INVENTION

The present invention relates generally to therapeutic and diagnostic applications of radiolabeled synthetic compounds, which are effective to (1) kill cancer cells undergoing DNA replication or repair by incorporation into the growing DNA strand resulting in DNA double strand breaks, (2) specifically target two membrane proteins expressed in cancer cells and implicated in tumorogenesis, butyrylcholinesterase (BChE) and the androgen receptor (AR). The compounds of the invention are taken up selectively by malignant tumor cells and are incorporated into the nucleus of such cells, where they produce a cytotoxic effect and/or are detectable via nuclear medicine imaging techniques.

The main treatments for ovarian, breast, prostate and many other cancers are surgery, chemotherapy and radiation therapy. In some cases a combination of two or more of these treatments is recommended. Typically, clinical trials for advanced carcinomas use combination chemotherapy based on established anti-cancer agents. For example, there are numerous active clinical trials (Phase I) dealing with recurrent and progressive ovarian carcinoma that rely on existing drugs such as paclitaxel, carboplatin, cisplatin, floxouridine and similar drugs in a combination chemotherapy. Many of these include autologous stem cell support to combat the side effects brought on by the administration of these drugs. Newer drugs include matrix metalloproteinase inhibitors, vaccines, and antibodies.

The prognosis is relatively poor for patients diagnosed with high-grade gliomas with glioblastoma multiforme patients rarely surviving beyond 12 months. The standard treatments for brain gliomas entail a multifaceted approach providing radiation, surgery, and chemotherapy. Many chemotherapeutic approaches are ineffective, due to the inability of most chemotherapeutics to cross the blood-brain barrier, and/or are overly toxic. Moreover, there is little ability to prevent recurrence following surgical resection. Palliative therapies for glioma besides radiation therapy and surgical resection include Avastin (bevacizumab) and temozolomide in combination with radiation therapy.

Many of the currently available front-line and salvage agents used in cancer therapy are associated with cumulative and/or irreversible toxicities that pose challenges for long-term treatment planning. The irreversible effects associated with some of these therapies include development of multidrug resistance, neurotoxicity, and nephrotoxicity. All of these diminish the probability of improved responses when multiple treatments are needed to keep the cancer under control.

It has previously been proposed to use targeted cytotoxic radioisotopes for the treatment and diagnosis of cancer. One of the intended benefits of targeted therapy is to diminish the incidence and severity of side effects by confining toxic exposure, more or less, to the disease site. Certain radioisotopes, particularly Auger electron-emitting isotopes, such as ¹²³I and ¹²⁵I are known to very toxic to viable cells, but only if they are localized within the nucleus of the cell (Wafters et al., Curr. Top. Stop Rad. Res., 12:389 (1977)). It has been reported that 5′-iodo-2′-deoxyuridine (IUdR), when labeled with the Auger electron emitter ¹²³I or ¹²⁵I exhibits substantial toxicity in mammalian cells in vitro (Makrigiorgos et al., Radiat. Res., 118:532-44 (1989)) and produces a therapeutic effect in animal tumor models (Baranowska-Kortylewicz et al., Int. J. Radiat. Oncol. Biol. Phys., 21:1541-51 (1991)). Furthermore, radiolabeled IUdR has been found to enable scintigraphic detection of animal and human tumors (Baranowska-Kortylewicz, supra). See also U.S. Pat. Nos. 5,094,835 and 5,308,605.

Considerable effort has been devoted to developing antibodies for the targeted delivery of therapeutic and diagnostic agents. However, antibodies themselves have not been capable of reaching the cell nucleus in effective amounts. Most such antibodies react with the cell surface, and are gradually internalized, routed to lysosomes and degraded (Kyriakos et al., Cancer Res., 52:835 (1992)). Degradation products, including any radioisotopes attached thereto, then gradually leave the cell by crossing the lysosomal membrane and then the cell membrane. Although a conventional radioisotope label on an antibody degradation product can theoretically pass through the nuclear membrane and deliver some radioactivity to the nucleus (Woo et al., WO 90/03799) actual observations show that the amount is limited, and in any event, is insufficient to have a toxic effect on tumor cells.

Protein and polypeptide hormones and growth factors, particularly those having cell surface receptors, may be directly radiolabeled and used to target a tumor cell. As in the case of targeting radiolabeled antibodies, however, radioisotopes bound to amino acid residues of hormones, growth factors and the like exit from the cell after catabolism, and do not appreciably bind to nuclear material.

U.S. Pat. No. 7,220,730, which is commonly owned with this application, relates to cancer specific radiolabeled conjugates useful as both therapeutic and diagnostic agents in the treatment of cancer. The radiolabeled conjugates incorporate a component that is effective to target tumor cells, which cells selectively take up and degrade the conjugate. The unmasked radioisotopic moiety is then delivered to the nucleus and incorporated into the nuclear material so as to produce a cytotoxic effect and/or render the cell detectable to nuclear medicine imaging.

Despite the many advances in the field of cancer therapy and diagnosis, a need still exists for innovative cancer treatment and diagnostic methods, which can be safely applied in a repetitive, long-term regimen, without the side effects produced by existing treatments. This is especially true with respect to therapeutic modalities for cancers that have high instances of relapse.

### SUMMARY OF THE INVENTION

The above-noted need is satisfied by the compounds of the present invention which are capable of binding to and being selectively taken up and degraded by a tumor composed of cancer cells having certain markers, and thereby delivering to the cell nucleus a radioisotope capable of being incorporated into the nuclear material, so as to produce a cytotoxic effect and/or to render the tumor cell detectable by nuclear medicine imaging. The compounds of the invention can be safely administered in long-term cancer treatments, without producing significant adverse health effects.

In accordance with one aspect of the present invention, there is provided a method of treatment of a tumor comprising cancer cells in a patient by administrating a therapeutically effective amount of a compound of the formula:

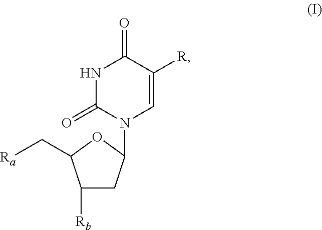

wherein Rₐ represents OH or:

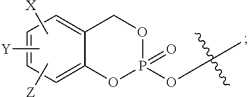

X represents H, F, Cl, or a C₁-C₈ alkyl, or C₁-C₈ alkoxy group;

Y represents H, C₁-C₈ alkyl, C₅-C₁₄ aryl, or a C₅-C₁₄ aryloxy group;

Z represents H, C₁-C₈ alkyl, C₅-C₁₄ aryl, or a C₅-C₁₄ aryloxy group;

R represents halogen, radiohalogen, or a C₁-C₈ alkyl, C₅-C₁₄ aryl, C₁-C₈ alkylthio, C₁-C₈ alkoxy, C₂-C₆ alkenyl, C₂-C₆ alkynyl, C₃-C₁₂ cycloalkyl, or Sn(C₁-C₄ alkyl)₃ group;

R_{b} represents halogen, radiohalogen, or a C₁-C₆ alkoxy or C₁-C₈ alkanoate group, an androgen receptor binding ligand linked to the compound via a cleavable linking moiety, or:

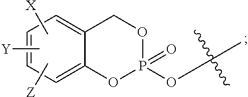

any of said alkyl, alkenyl, alkynyl, alkylthio, alkoxy and cycloalkyl group being optionally substituted by at least one halogen, OH, SH, NH₂, C₁-C₄ monoalkylamino, C₁-C₄ dialkylamino, COOH, CN, NO₂, C₁-C₄ alkyl, C₁-C₄ alkoxy or phenyl group, any of said aryl, aryloxy, and phenyl group being optionally substituted by at least one halogen, OH, SH, NH₂, C₁-C₄ monoalkylamino, C₁-C₄ dialkylamino, COOH, CN, NO₂, C₁-C₄ alkyl or C₁-C₄ alkoxy group; said radiohalogen represents ¹²³I, ¹²⁴I, ¹²⁵I, ¹³¹I, ²¹¹At, ¹⁸F, ⁷⁶Br, ⁷⁷Br, or ^{80m}Br; stereoisomeric forms and pharmaceutically acceptable salts of said at least one compound; and

with the proviso that at least one of the Rₐ and R_{b} substitutents represents:

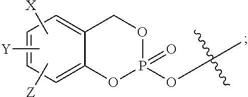

and

the wavy line indicating the point of attachment to the ribose moiety.

In another aspect of the invention, the compounds of this invention can be administered as either fast or slow eluting isomers or a mixture thereof, as will be explained below in further detail.

The compounds of this invention can be used to eradicate residual cancer cells, e.g. in relapsing cancers, with minimal, if any, damage to normal tissues or to tissues in and around the treatment site. The method of the present invention may be applied for treating and diagnosing cancers comprising cells characterized by at least one of BChE expression and androgen binding affinity, including, without limitation, ovarian, breast, prostate, head and neck, pancreatic, glioma, colorectal, and meningioma malignant tumors.

The compounds of the present invention have been designed so as to take advantage of three characteristics of many relapsing cancers, i.e. (1) relapsed/advance cancers have a large portion of rapidly growing and dividing cells (i.e. a large S-phase fraction); (2) AR is expressed in practically all prostate cancer (primary and metastatic), ovarian cancer (>90% positive for AR regardless of the tumor site) and breast cancer (even when estrogen receptor (ER)-negative and progesterone receptor (PR)-negative, breast cancer cells express AR), glioma, head and neck cancer, colorectal cancer, and meningiomas; and (3) BChE is expressed in a variety of cancer types allowing for malignant tumor targeting and avoidance of impacting surrounding healthy tissue thereby enabling the compounds of the invention to be used to treat non-resectable malignant tumors.

The compounds of formula (I), above, can also be used to advantage for diagnosis of malignant tumors. The method comprises administering to a patient a diagnostically effective amount of labeled compound of formula (I), and then imaging the tumor by scintigraphic imaging or magnetic resonance spectroscopy. This method can also be adapted for use in monitoring tumor activity in a subject.

Also in accordance with the invention there is provided a compound of the formula:

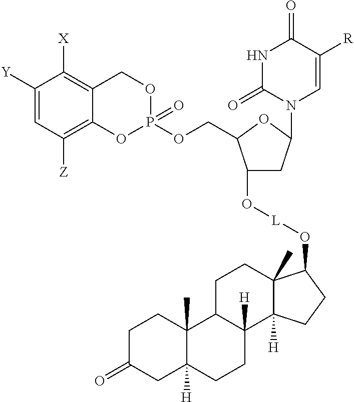

wherein X represents H, F, Cl, or a C₁-C₄ alkyl, or C₁-C₄ alkoxy group;

Y represents H or a C₁-C₄ alkyl group;

Z represents H or a C₁-C₄ alkyl group;

R represents halogen, radiohalogen, or a C₁-C₄ alkyl, C₆-C₁₄ aryl, C₁-C₈ alkylthio, C₁-C₈ alkoxy, C₂-C₆ alkenyl, C₂-C₆ alkynyl, C₃-C₁₂ cycloalkyl, or Sn(C₁-C₄ alkyl)₃ group;

any of said alkyl, alkenyl, alkynyl, alkylthio, alkoxy, and cycloalkyl group being optionally substituted by at least one halogen, OH, SH, NH₂, C₁-C₄ monoalkylamino, C₁-C₄ dialkylamino, COOH, CN, NO₂, C₁-C₄ alkyl, C₁-C₄ alkoxy or phenyl group, any of said aryl and phenyl group being optionally substituted by at least one halogen, OH, SH, NH₂, C₁-C₄ monoalkylamino, C₁-C₄ dialkylamino, COOH, CN, NO₂, C₁-C₄ alkyl or C₁-C₄ alkoxy group; L is a cleavable bifunctional linking moiety; and said radiohalogen represents ¹²³I, ¹²⁴I, ¹²⁵I, ¹³¹I, ²¹¹At, ¹⁸F, ₇₆Br, ⁷⁷Br, or ^{80m}Br; and stereoisomeric forms and pharmaceutically acceptable salts of said compound.

The cytotoxic effects of the methods of the invention are induced only when one or more of the compounds described herein are incorporated into the DNA of rapidly dividing tumor cells. This dependence of radiotoxicity on the participation of the compound in DNA synthesis, in combination with relatively rapid pharmacokinetics, limits the exposure of normal tissue to radiation. In other words, the compound(s) that remain(s) in systemic circulation, or enter(s) normal tissue or organs, is (are) essentially innocuous. Accordingly, the compounds of the invention may be administered frequently and without appreciable adverse effects.

A kit comprising a vessel containing a compound of formula (I), above, and a pharmaceutically acceptable carrier medium is also provided.

### BRIEF DESCRIPTION OF THE DRAWINGS

FIG. 1 shows HPLC traces of the (a) fast and (b) slow isomers of 5-[¹²⁵I]-iodo-5′-O-cycloSaligenyl-2′-deoxyuridine monophosphate. (a) HPLC analysis: separated diastereomer (6b fast), t_{R}=29.8 min (≧98% pure, Bioscan NaI(T)); injection: 14.4 μCi in 15 μL of 50% MeCN; column: Jupiter C18, 300 Å(5 μm, 4.6×250 mm); eluent: solvent A 10% MeCN in water, solvent B MeCN; eluted at 0.8 mL/min with a linear gradient of B from 0-20% over 33 min, then a linear gradient of B from 20-95% for 5 min, and 95% B kept 15 min. (b) HPLC analysis: separated diastereomer (6b slow), t_{R}=30.8 min (≧98% pure, Bioscan NaI(T)); injection: 21.2 μCi in 25 μL of 50% MeCN; column: Jupiter C18, 300 Å (5 μm, 4.6×250 mm); eluent: solvent A 10% MeCN in water, solvent B MeCN; eluted at 0.8 mL/min with a linear gradient of B from 0-20% over 33 min, then a linear gradient of B from 20-95% for 5 min, and 95% B kept 15 min.

FIG. 2 shows HPLC traces of the (a) fast and (b) slow isomers of 5-[¹²⁵I]-iodo-5′-O-cyco(3-methylSaligenyl)-2′-deoxyuridine monophosphate. HPLC analysis: separated diastereomers (a) 7b fast, t_{R}=25.3 min and (b) 7b slow t_{R}=26.8 min (≧98% pure, Bioscan NaI(T)); injection: 10.5 μCi in 20 μL of 50% MeCN; column: Jupiter C18, 300 Å (5 μm, 4.6×250 mm); eluent: 20% MeCN in water, eluted at 0.8 mL/min for a period of 45 min.

FIG. 3 shows HPLC traces of the (a) fast and (b) slow isomers of 5-[¹²⁵I]-iodo-5′-O-[cyclo-3,5-di(tert-butyl)-6-fluoroSaligenyl]-2′-deoxyuridine monophosphate. HPLC separation of diastereomers: (a) 8b fast t_{R}=30.3 min and (b) 8b slow t_{R}=32.8 min (≧98% pure, Bioscan NaI(T)). Complete separation of diastereomers in a single HPLC run was achieved if the injection of 8b was ≦220 μCi. Column: ACE C18, 100 Å (5 μm, 4.6×250 mm); eluted at 0.8 mL/min with 45% MeCN in water. The co-injected mixtures of corresponding diastereomers: 8 fast/8 slow (˜10 μg) and radioiodinated 8b fast/8b slow (˜30 μCi) in 25 μL of 50% MeCN were analyzed, monitoring radioactivity (Bioscan NaI(T)) and UV at 280 nm.

FIG. 4 shows an HPLC trace demonstrating the separation of the fast and slow isomers of 5-[¹²⁵I]-iodo-5′-O-[cyclo-3,5-Di-(tert-butyl)-6-fluoroSaligenyl]-3′-fluoro-2′,3′-dideoxyuridine. HPLC analysis: the separated and dried product residue was reconstituted in dry MeCN (˜1 μCi/μL) and 12 μCi (10 μL) injected on an analytical column. The analysis showed 24b fast at t_{R}=31.0 min and 24b slow; t_{R}=31.9 min (≧98% pure, Bioscan NaI(T)). Column: ACE C18, 100 Å (5 μm, 4.6×250 mm); eluent: solvent A 50% MeCN in water, solvent B MeCN; eluted at 1.0 mL/min with a linear gradient of B from 0-95% over a period of 90 min.

FIG. 5 shows an HPLC trace demonstrating the separation of the fast and slow isomers of 5-O-[cyclo-3,5-Di-(tert-butyl)-6-fluoroSaligenyl]-3′-deoxy-3′-fluorothymidine monophosphate. HPLC analysis: a mixture of diastereomers 23 fast, t_{R}=45.5 min; 23 slow, t_{R}=47.0 min (≧97% pure, UV at 280 nm); column: ACE C18, 100 Å (5 μm, 4.6×250 mm); eluent: solvent A 50% MeCN in water, solvent B MeCN; eluted at 0.8 mL/min with solvent A for 60 min (isocratic) and then a linear gradient of B from 0-95% B over 30 min.

FIG. 6 shows clearance curves for human colorectal cancer LS174T xenografts extirpated from athymic mice injected simultaneously with 6b and ¹³¹IUdR. FIG. 6A shows the clearance curves for 6b slow and ¹³¹IUdR. FIG. 6B shows the clearance curves of 6b fast and ¹³¹IUdR.

FIG. 7 shows planar images acquired 24 h, 48 h, and 72 h after IV administration of 6b as a diastereomeric mixture in athymic mice bearing subcutaneous human colorectal adenocarcinoma xenografts LS174T. The planar images were taken with a Technicare gamma camera.

FIG. 8 shows planar images of female athymic mice with subcutaneous human colorectal adenocarcinoma LS174T xenografts acquired 24 h, 96 h, and 120 hours after IV administration of 8b fast. The planar images were taken with a Technicare gamma camera.

FIG. 9 is a set of graphs comparing blood, tumor, and several normal tissue clearance curves for 8b fast.

FIG. 10 is a set of graphs comparing blood, tumor, and several normal tissue clearance curves for 8b slow.

FIG. 11 is a (A) graphical representation of the weights of solid tumors and tumor cells recovered in ascites of OVCAR-3 bearing mice seven weeks after the first dose of the radiolabeled diastereomeric mixture of compounds 6b (6b mix), 7b (7b mix), or the parent compound ¹²⁵IUdR, and (B) graphical representation of the whole body radioactivity of the mice two weeks after treatment.

FIG. 12 is a graphical representation of the retention of radioactivity in solid tumor deposits and in the cancer cells in ascites 42 days after the administration of the radioactive compounds 6b mix, 7b mix, and the parent compound ¹²⁵IUdR.

FIG. 13 shows weights of tumors recovered during the necropsy of OVCAR-3-bearing athymic mice conducted seven weeks after tumor implant and treated with fractionated doses of 8b slow.

FIG. 14 is a set of dose response curves for the and solid tumor burden (A) and cancer cells in ascites fluid (B) in OVCAR-3-bearing athymic mice treated with fractionated doses of 8b slow.

FIG. 15 is the summary of the hematological values in OVCAR-3-bearing mice treated with fractionated doses of 8b slow. Hemoglobin and hematocrit values in athymic mice bearing IP OVCAR-3 tumor implants 48 days after treatment with the fractionated doses of 8b slow.

FIG. 16 is a graphical representation of the results of statistical analyses of tumor burden and hematological parameters in the fractionated therapy study. Bolded values indicate the statistically significant differences, i.e. P<0.05.

FIG. 17 is a graphical representation of OVCAR-3 tumor weights obtained during post-therapy necropsy. The left panel shows the weight of solid tumor deposits. The right panel shows the weight of the cancer cell pellet recovered with the peritoneal lavage. There is a demonstrated dose-dependent response to 8b slow.

FIG. 18 is a graphical representation of in vitro kinetics of 7b uptake in OVCAR-3 human adenocarcinoma cells. The average radioactive concentration in each well was 0.74±0.04 μCi/mL (27.3±1.4 kBq/mL).

FIG. 19 is a graphical representation of in vitro kinetics of 7b uptake in LS174T human colorectal cancer cells. The average radioactive concentration in each well was 0.76±0.06 μCi/mL (28.2±2.2 kBq/mL).

FIG. 20 is a graphical representation of changes in the surviving fraction of LS174T cells grown with 6b fast and 6b slow. Clonogenic assay was performed on cells exposed to radioactive compounds for 4 h followed by additional 24 h culture in nonradioactive media before the cell harvest and plating at densities suitable for clonogenic assay.

FIG. 21 is a graphical representation of the surviving fraction of LS174T colorectal adenocarcinoma cells treated with 7b fast and 7b slow for 4 hours at a compound concentration of 5 μCi/mL (185 kBq/mL). Cells were harvested immediately after the exposure to the compound and re-plated at densities suitable for the clonogenic assay.

FIG. 22 is a graphical representation of the subcellular distribution of 6b mix in OVCAR-3 cancer cells measured at 1 hour and 24 hours.

FIG. 23 is a graphical representation of the surviving fraction of U-87 human glioblastoma cells treated with 6b slow and 6b fast at 37 kBq/mL for 24 hours (bars represent average; capped lines are standard deviation).

FIG. 24 is a graphical representation of concentration-dependent survival of U-87 human glioblastoma cells treated with 6b as isolated fast and slow isomers.

FIG. 25 is a graphical representation of cellular uptake and subcellular distribution of 6b in U-87 human glioblastoma cells after 24 hour exposure to 6b as isolated fast and slow isomers.

FIG. 26 is a graphical representation of the subcellular distribution and retention of 6b fast and 6b slow in DNA of U-87 human glioblastoma cells.

FIG. 27 is a graphical representation of the concentration-dependent uptake of 6b, as isolated fast and slow isomers, by U-87 human glioblastoma cells.

### DETAILED DESCRIPTION OF THE INVENTION

The compounds of formula (I), above, are composed of one component which is effective for killing cancer cells undergoing rapid DNA replication in addition to one or more specific targeting components capable of targeting BChE and/or AR expressing malignant tumor cells. Additionally, these compounds bind sex-hormone binding globulin (SHBG), which increases their half-life in the serum and allows uptake of the compounds in the cell via the SHBG receptor

Iodine-125, for example, damages DNA and efficiently kills cells only when it is located in the cell nucleus near or within DNA. The use of this radioisotope is beneficial because it is practically harmless when present in extracellular spaces. Thus, these compounds, if used alone or in combination therapies, will not increase the overall toxicity of the primary treatment.

Thymidine analogs, such as IUdR, when radiolabeled with an Auger emitter, are essentially innocuous outside the cell and ineffective at killing cells inside the cytoplasm. IUdR may also be radiolabeled with alpha-and beta-emitters. Unlike Auger electron emitters, these radioisotopes are radiotoxic even when outside the cell. Such isotopes would allow for the irradiation of neighboring cells, i.e., a bystander effect, which is beneficial, particularly if AR, BChE, and/or SHBG expression is not uniform. IUdR is, for the most part, taken up selectively by dividing malignant tumor cells. These cells are located within a niche of nondividing cells and the radioactive compound(s) can be indefinitely retained within the nucleus of the cancer cell following DNA incorporation. Nondividing cells will not incorporate radiolabeled IUdR into their DNA and most of the radiolabeled IUdR that is not taken up by cancerous cells will be catabolized/dehalogenated rapidly (t_{1/2} measureable in minutes) and thus, will not incorporate in the DNA of distant non-cancerous dividing cells.

Furthermore, since radiolabeled IUdR is a small molecule it will not induce an immune response, which permits repeated injections, continuous infusion, or similar modes of administration. In order to provide cancer cell specificity and enhanced delivery, in certain embodiments of this invention the radiolabeled thymidine analogs are conjugated to a BChE selective ligand. In other embodiments of this invention, the radiolabeled thymidine analogs are conjugated to an AR specific ligand. Additionally, in other embodiments of this invention, the radiolabeled thymidine analogs are conjugated to both a BChE and an AR ligand.

Radiolabeled IUdR, in one embodiment of this invention, may be chemically coupled to a cycloSaligenyl phosphotriester moiety having binding affinity for BChE. BChE plays a role in tumorigenesis and is expressed predominantly on the membrane and in the cytosol of many malignant tumor cells. BChE genes are amplified, mutated, and/or aberrantly expressed in a variety of human tumor types. BChE contains the consensus peptide motif S/T-P-X-Z, which is found in many substrates of cdc2-related protein kinases suggesting that phosphorylation by cdc2-related kinases may be the molecular mechanism linking BChE to tumor proliferation. Studies have also demonstrated BChE upregulation in brain tumors (Rios, et al., Surg Neurol 55:106-12 (2001)), ovarian carcinomas (Zakut et al., J. Clin. Invest. 86:900-908 (1990)), breast cancer (Bernardi et al., Cancer Genetics and Cytogenetics 197:158-165 (2010)), and glioma (Saez-Valero et al., 206:173-176 (1996)).

The term “differential butyrylcholinesterase expression” as used herein, refers to at least one recognizable difference in protein or nucleic acid expression. It may be a quantitatively measureable, semi-quantitatively estimatable or qualitatively detectable difference in cells, tissue, or bodily fluid protein expression. Thus, differentially expressed butyrylcholinesterase may be strongly expressed in cells, tissue, or bodily fluid in the normal state and less strongly expressed or not expressed at a measureable level in the damaged state. Conversely, it may be strongly expressed in cells, tissue, or bodily fluid in the damaged state, and less strongly expressed or not expressed at all in the normal state.

Radiolabeled IUdR, in another embodiment, may be conjugated to an androgen receptor binding ligand such as 4-dihydrotestosterone (DHT; also known as 17β-hydroxy-5α-androstan-3-one, 4,5α-dihydrotestosterone, androstanolone, stanolone). Importantly, AR is expressed on cells from a variety of cancers, such as 50-90% of breast tumors (Bryan, R. M., et al. (1984) Cancer, 54:2436-2440; Lea, O. A., et al. (1989) Cancer Res., 49:7162-7167; Soreide, J. A., et al. (1992) Eur. J. Surg. Oncol., 18:112-118). DHT, in addition to providing specific targeting of the compound to cells expressing AR, has also demonstrated anti-cancer effects in breast cancer experimental models (see, for example, Poulin, R., et al. (1988) Breast Cancer Res. Treat., 12:213-225) and other androgens, such as fluoxymesterone, have produced anti-cancer effects in administration to patients (see, for example, Ingle, J. N., et al. (1991) Cancer, 67:886-891).

Preferred compounds in accordance with the present invention have the formula:

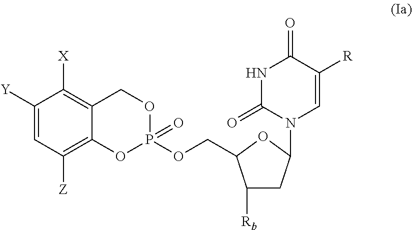

wherein X represents H, F, Cl, or a C₁-C₄ alkyl, or C₁-C₄ alkoxy group;

Y represents H or a C₁-C₄ alkyl group;

Z represents H or a C₁-C₄ alkyl group;

R represents halogen, radiohalogen, or a C₁-C₄ alkyl, C₁-C₄ alkoxy or phenyl group;

R_{b} represents halogen, radiohalogen, OH or C₁-C₄ alkoxy, or an androgen receptor binding ligand linked to the compound via a cleavable linking moiety.

In formula (Ia), any of the alkyl, alkoxy and phenyl group is optionally substituted by at least one halogen, OH, SH, NH₂, C₁-C₄ monoalkylamino, C₁-C₄ dialkylamino, COOH, CN, NO₂, C₁-C₄ alkyl or C₁-C₄ alkoxy group; and the radiohalogen represents ¹²³I, ¹²⁴I, ¹²⁵I, ¹³¹I, ²¹¹At, ¹⁸F, ⁷⁶Br, ⁷⁷Br, or ^{80m}Br; and stereoisomeric forms and pharmaceutically acceptable salts thereof.

It should be appreciated that compounds of formula (I) and (Ia), above, may have one or more asymmetric centers and thus exist as stereoisomers, including diastereomers, with stereocenters named according to the Cahn-Ingold-Prelog system (R/S designation of stereocenters). Although the structural formulas set forth above are represented without regard to stereochemistry, it is intended to include all possible stereoisomers, which may be diastereomeric mixtures, as well as resolved, substantially pure optically active and inactive forms, and pharmaceutically acceptable salts thereof.

Stereoisomers of the compounds used in the practice of this invention can be selectively synthesized or separated into pure, optically-active or inactive form using conventional procedures known to those skilled in the art of organic synthesis. For example, mixtures of stereoisomers may be separated by standard techniques including, but not limited to, resolution of diastereomeric forms, normal, reverse-phase, and chiral chromatography, preferential salt formation, recrystallization, and the like, or by asymmetric synthesis either from enantiomerically or diastereomerically pure starting materials or by deliberate synthesis of target enantiomers or diastereomers. All of the various isomeric forms of the compounds of formulas (I) and (Ia), above, are within the scope of this invention. Nonstereoselective syntheses produce the diastereometric mixture of cycloSaligenyl-phosphotriesters. Isomers may be separated by reverse phase HPLC and resolved according to their retention time as the slow and the fast diastereomers, as described in further detail below. The slow diastereomers are more potent inhibitors of BChE in contrast to the fast diastereomers.

The phrase “enantiomeric excess” or “ee” is a measure, for a given sample, of the excess of one enantiomer over a racemic sample of a chiral compound and is expressed as a percentage. Enantiomeric excess is defined as 100*(er−1)/(er+1), where “er” is the ratio of the more abundant enantiomer to the less abundant enantiomer.

The phrase “diastereomeric excess” or “ee” is a measure, for a given sample, of the excess of one diastereomer over a sample having equal amounts of diastereomers and is expressed as a percentage. Diastereomeric excess is defined as 100*(dr−1)/(dr+1), where “dr” is the ratio of a more abundant diastereomer to a less abundant diastereomer. The term does not apply if more than two diastereomers are present in the sample.

Preferably, where the “substantially pure” compound of formula (I), above, is provided as a diastereomer, the diastereomer is present at an diastereomeric excess of greater than or equal to about 80%, more preferably, at an diastereomeric excess of greater than or equal to about 90%, more preferably still, at an diastereomeric excess of greater than or equal to about 95%, more preferably still, at an diastereomeric excess of greater than or equal to about 98%, most preferably, at an diastereomeric excess of greater than or equal to about 99%.

As used herein, the term “alkyl” refers to saturated straight and branched chain hydrocarbon radicals, having 1-8 and preferably 1-4 carbon atoms. The term “alkenyl” is used to refer to unsaturated straight and branched chain hydrocarbon radicals including at least one double bond, and having 2-6. Such alkenyl radicals may be in trans (E) or cis (Z) structural configurations. The term “alkynyl” is used herein to refer to both straight and branched unsaturated hydrocarbon radicals including at least one triple bond and having 2-6.

The term “cycloalkyl” as used herein refers to a saturated cyclic hydrocarbon radical with one or more rings, having 3-12.

Any alkyl, alkenyl, alkynyl or cycloalkyl moiety of a compound described herein may be substituted with one or more groups, such as halogen, OH, SH, NH₂, C₁-C₄ monoalkylamino, C₁-C₄ dialkylamino, COOH, CN, NO₂, C₁-C₄ alkyl or C₁-C₄ alkoxy.

The term “aryl” as used herein refers to an aromatic hydrocarbon radical composed of one or more rings and having 5 or 6-14 carbon atoms and preferably 5 or 6-10 carbon atoms, such as phenyl, naphthyl, biphenyl, fluorenyl, indanyl, or the like. Any aryl moiety of a compound described herein may be substituted with one or more groups, such as halogen, OH, SH, NH₂, C₁-C₄ monoalkylamino, C₁-C₄ dialkylamino, COOH, CN, NO₂, C₁-C₄ alkyl or C₁-C₄ alkoxy. The aryl moiety is preferably substituted or unsubstituted phenyl.

The term “halogen” or “halo” as used herein refers to Fl, Cl, Br and I.

The term “radiohalogen” as used herein refers to an isotopic form of halogen that exhibits radioactivity. The radiohalogen is preferably selected from the group consisting of ¹²³I, ¹²⁴I, ¹²⁵I, ¹³¹I, ²¹¹At, ¹⁸F, ⁷⁶Br, ⁷⁷Br, and ^{80,}Br.

The term “alkoxy” refers to alkyl-O—, in which alkyl is as defined above.

The term “alkylthio” refers to alkyl-S—, in which alkyl is as defined above.

The term “carboxy” refers to the moiety —C(═O)OH.

The term “aryloxy” refers to the moiety —O-aryl, in which aryl is defined above.

The term “alkanoate” refers to the moiety —O—C(═O)-alkyl, in which alkyl is as defined as above.

The term “monoalkylamino” refers to the moiety —NH(alkyl), in which alkyl is as defined as above.

The term “dialkylamino” refers to the moiety —N(alkyl)₂, in which alkyl is as defined as above.

The cleavable linking moiety, L, can be a diester or a phosphate. The diester moiety may be represented as —O—C(═O)—(CH₂)ₙ—C(═O)—O—, wherein n=2−10. The preferred cleavable linking moiety is a succinate moiety.

The term “androgen receptor binding ligand”, is defined as an androgen receptor agonist or an androgen receptor antagonist.

The androgen receptor agonists that may be used in accordance with the present invention includes, without limitation, 4-dihydrotestosterone (DHT), testosterone, mibolerone, methyltrienolone, and methyltestosterone. The androgen receptor antagonists that may be used in accordance with the present invention includes, without limitation, hydroxyflutamide, flutamide, cyproterone acetate, spironolactone, ketoconazole, and finasteride. In accordance with the present method of the invention, synthetic modification of known androgen receptor agonists and antagonists, to allow for linkage to the compounds used in the method of the invention, would be well understood by a person having ordinary skill in synthetic organic chemistry. The preferred ligand is DHT bound through its hydroxyl substituent.

Based on BChE inhibition studies (IC₅₀), structure-activity relationships (SAR) have elucidated key structural features providing enhanced BChE selectivity and inhibition. By appropriate selection of the X substituent of the compound of formula (Ia), above, the rate of hydrolysis of the cycloSal-phospho-triester moiety can be varied. For example, the possibilities for X could include hydrogen, methyl, chloro, fluoro, or methoxy radicals. Increased BChE inhibitory activity and enhanced selectivity for BChE among slow/fast diastereomers at the phosphorus atom is achieved through selection of a tert-butyl group on the cycloSal-phospho-triester phenyl ring as the Y and Z substituent. There is also increased BChE inhibitory activity and enhanced selectivity for BChE among slow/fast diastereomers when R is iodide. Increased BChE inhibitory activity has also been observed for the compound of formula (I), above, when R_{b} is a fluoride.

Accordingly, particularly preferred are the compounds of formula (Ia), above, wherein the possibilities for X, Y, and Z on the cycloSal-phospho-triester phenyl ring are as follows: X═H or F, Y═H or tert-butyl, and Z═H, CH₃ or tert-butyl. Preferred also are the compounds of formula (I) wherein R═CH₃ or ¹²⁵I and R_{b}=O-L-DHT, OH, or F. The slow diastereomers also represent the preferred isomers of the present invention.

The term “pharmaceutically acceptable salts” as used herein refers to salts derived from non-toxic, physiologically compatible acids and bases, which may be either inorganic or organic. Useful salts may be formed from physiologically compatible organic and inorganic bases, including, without limitation, alkali and alkaline earth metal salts, e.g., Na, Li, K, Ca, Mg, as well as ammonium salts, and salts of organic amines, e.g., ammonium, trimethylammonium, diethylammonium, and tris-(hydroxymethyl) methylammonium salts. The compounds of the invention also form salts with organic and inorganic acids, including, without limitation, acetic, ascorbic, lactic, citric, tartaric, succinic, fumaric, maleic, malonic, mandelic, malic, phthalic, salicyclic, hydrochloric, hydrobromic, phosphoric, nitric, sulfuric, methane sulfonic, naphthalene sulfonic, benzene sulfonic, para-toluene sulfonic and similar known, physiologically compatible acids. In addition, when a compound of Formula I contains both a basic moiety and an acidic moiety, zwitterions (“inner salts”) may be formed and are included within the term “salt(s)” as used herein.

The compounds of the invention, as stated hereinabove, may be administered alone, containing both therapeutic and diagnostic moieties, or alternatively, as two compounds with one compound acting as a therapeutic and a second compound acting as a diagnostic. The two compounds could be coadministered concurrently or sequentially. These compounds may be administered as separate dosage units or formulated for administration together, according to procedures well known to those skilled in the art. See, for example, Remington: The Science and Practice of Pharmacy, 20^{th} ed., A. Genaro et al., Lippencot, Williams & Wilkins, Baltimore, Md. (2000).

Therapeutic preparations comprising the compounds of this invention may be conveniently formulated for administration with a biologically acceptable vehicle, which may include the patient's own serum or serum fractions. Other suitable vehicles include liposomes and similar injectable suspensions, saline, activated carbon absorbents, and solutions containing cyclodextrins such as alphadex and betadex. Additionally, IUdR analogs may be derivatized, e.g. by esterification of available hydroxyl groups, with long chain fatty acids to increase the circulation half-life of the compounds. The concentration for diagnostic uses of the compound in the chosen vehicle should normally be from about 0.1 mCi/mL to about 10 mCi/mL. The concentration for therapeutic uses of the compound in the chosen vehicle should normally be from about 1 mCi/mL to about 100 mCi/mL. These concentrations may vary depending on whether the method of administration is intravenous, intraperitoneal, or intratumoral, which are the preferred routes of administration. In all cases, any substance used in formulating a therapeutic or diagnostic preparation in accordance with this invention should be virus-free, pharmaceutically pure and substantially non-toxic.

For therapeutic applications, the compound will typically be administered in a therapeutically effective amount, which will normally be a dose that provides from about 1 mCi (37 MBq)−20 mCi (740 MBq) of radioactivity per 24 hours. A diagnostically effective amount of the compound administered for diagnostic applications will generally be an amount sufficient to provide between 0.1 mCi and 10 mCi of radioactivity. For the determination of AR expression, the imaging can commence immediately after the administration. To detect DNA uptake, imaging may begin 1 hour after administration. Notably, when using longer lived radioisotopes, imaging can occur at least daily for 7 days or longer to assess the tumor growth kinetics. The determination of an appropriate dose of the compound, either therapeutic or diagnostic, for a particular patient will, of course, be determined based on the type and stage of the patient's cancer and the judgment of the attending medical oncologist or radiologist, as the case may be.

For both therapeutic and diagnostic applications, the compounds useful in the method of the invention can be imaged in vitro, ex vivo, and in vivo by using magnetic resonance spectroscopy or scintigraphic imaging depending upon the moiety attached to the compound which enables said imaging. For instance, by labeling a compound of the method of the invention with ^{[19]}F, the compound could be imaged in vitro, ex vivo, and in vivo using magnetic resonance spectroscopy (MRS). For methods of the invention that utilize radiolabeled compounds, those methods could utilize scintigraphic imaging techniques such as positron emission tomography (PET) or single photon emission computed tomography (SPECT).

As used herein, the expression “tumor activity”, refers to a tumor's presence, progression, regression or metastasis in a subject, or to a reduction of tumor size due to therapeutic intervention.

As used herein, the expression, “tumor size”, includes all methods of quantifying the size of a tumor which include, but are not limited to, weight, mass, and volume of the tumor ex vivo and in vivo. Therefore, “baseline tumor size”, as used herein, refers to the size of the tumor at or near the time of initial diagnosis, and prior to any form of treatment, so as to provide a starting point from which changes, or lack thereof, to the tumor's size can be quantified.

As used herein, the term “diagnosis” or “diagnostic”, includes the provision of any information concerning the existence, non-existence or probability of a malignant tumor or a tumor composed of cancer cells in a patient. It further includes the provision of information concerning the type or classification of the disorder or of symptoms which are or may be experience and in connection with it. It encompasses prognosis of the medical course of the condition.

If necessary, the action of contaminating microorganisms may be prevented by various anti-bacterial and/or anti-fungal agents, such as parabens, chlorbutinol, phenyl, sorbic acid, thimerosal and the like. It will often be preferable to include in the formulation isotonic agents, for example, glucose or sodium chloride. Additionally, free-radical scavengers and antioxidants such as ascorbic acid and the like may be employed to allow for a longer storage of the radioactive compound.

The compounds of the invention will typically be administered from 1-4 times a day, so as to deliver the above-mentioned daily dosage. However, the exact regimen for administration of the compounds and compositions described herein will necessarily be dependent on the needs of the individual subject being treated, the type of treatment administered and the judgment of the attending medical specialist. As used herein, the terms “patient” and “subject” include both humans and animals.

The compounds of the invention may be administered as such, or in a form from which the active agent can be derived, such as a prodrug. A prodrug is a derivative of a compound described herein, the pharmacologic action of which results from the conversion by chemical or metabolic processes in vivo to the active compound. Prodrugs include, without limitation, ester, acetal, imine, carbamate, succinate, and phosphate derivatives of the compounds of formula I, above. Other prodrugs may be prepared according to procedures well known in the field of medicinal chemistry and pharmaceutical formulation science. See, e.g., Lombaert et al., J. Med. Chem., 37: 498-511 (1994); and Vepsalainen, Tet. Letters, 40: 8491-8493 (1999).

As used herein, the expression “pharmaceutically acceptable carrier medium” includes any and all solvents, diluents, or other liquid vehicle, dispersion or suspension aids, surface agent agents, isotonic agents, thickening or emulsifying agents, preservatives, and the like as suited for the particular mode of administration desired. Remington: The Science and Practice of Pharmacy, 20^{th} edition, A. R. Genaro et al., Part 5, Pharmaceutical Manufacturing, pp. 669-1015 (Lippincott Williams & Wilkins, Baltimore, Md./Philadelphia, Pa.) (2000)) discloses various carriers used in formulating pharmaceutical compositions and known techniques for the preparation thereof. Except insofar as any conventional pharmaceutical carrier medium is incompatible with the compounds of the present invention, such as by producing an undesirable biological effect or otherwise interacting in an deleterious manner with any other component(s) of a formulation comprising such compounds, its use is contemplated to be within the scope of this invention. It is noted in this regard that administration of the compounds of this invention with any substance that competes therewith for BChE and/or AR binding is to be avoided.

The method of treating cancer described herein will normally include medical follow-up to determine the effectiveness of the compound(s) of formula (I), above, in eradicating the tumor in a patient undergoing treatment. The term “treatment”, as used herein, refers to methods of treating malignant tumors or tumors comprising cancer cells including surgical excision, chemotherapy, and/or radiation therapy.

Synthetic routes for the preparation of the compounds of formula (I) are exemplified hereinbelow.

The targeted delivery of radionuclides to cancer cells in the manner described herein produces strong cytotoxic activity, in that the radionuclide is introduced into the DNA of the multiplying cells, where it induces DNA strand breaks in the double helix. Moreover, by delivering radiolabeled agents to a specific site and relying on mechanisms operational at the site of delivery to release the radiolabeled agent, the usual in vivo degradation pathways are by-passed, bioavailability of the radiolabeled agent is improved and more tumor cells are exposed to the cell killing effect of the radiation as they enter into the S phase.

A kit comprising a vessel containing a compound of formula (I), above, and a pharmaceutically acceptable carrier medium is also provided. The kit may optionally include one or more of catheter tubing, syringe, antibacterial swabs, all antiseptically packaged, as well as instructions for practicing the above-described methods.

The following examples describe the synthesis of the compounds of the present method of the invention, as well as biological testing of certain of the compounds. These examples are provided for illustrative purposes only and are not intended to limit the scope of the invention in any way.

### EXAMPLE 1

### Synthesis of cycloSaligenyl Monophosphate Analogs of 5-[¹²⁵I]-iodo-2′-deoxyuridine (cycloSal-[¹²⁵I]IUdRMP) and 5-[¹²⁵I]iodo-3′-fluoro-2′,3′-dideoxyuridine(cycloSal-[¹²⁵I]FIUdRMP)

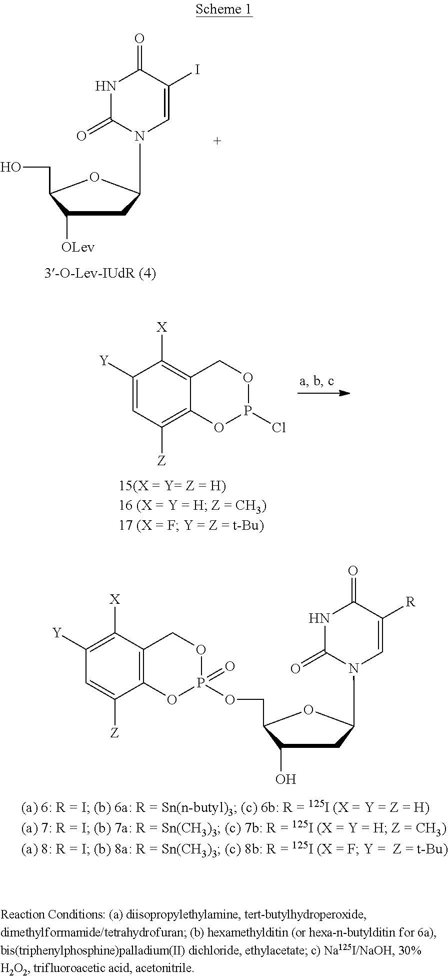

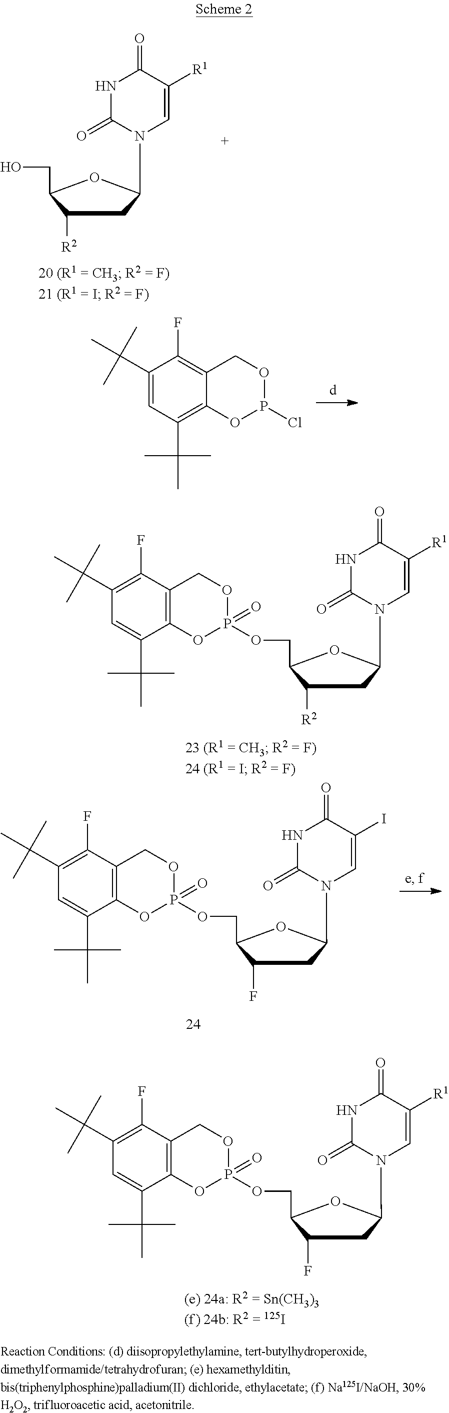

The cycloSaligenyl monophosphates of 5-[¹²⁵I]-iodo-2′-deoxyuridine (cycloSal-[¹²⁵I]IUdRMP) and 5-[¹²⁵I]iodo-3′-fluoro-2′,3′-dideoxyuridine (cycloSal-[¹²⁵I]FIUdRMP) were synthesized in consecutive steps, as shown in Schemes 1 and 2. Nonradioactive iodo-analogs were treated with hexamethylditin under palladium catalysis to provide the corresponding 5-trimethylstannyl cycloSaligenyl derivatives. These organotin compounds provided the starting materials for the target [¹²⁵I]-radioiodinated cycloSaligenyl phosphotriesters 6b-14b, and 24b. All radioiodolabelings, proceeding via electrophilic iododestannylation, were carried out at the non-carrier-added level.

The cycloSal-moiety was initially inserted into the scaffold as a chlorophosphite. Thus, 5-iodo-3′-O-levulinyl-2′-deoxyuridine 4, or optionally IUdR, was treated with cyclic chlorophosphites 15-17 (Scheme 1) in the presence of diisopropylethylamine. The reactions led to corresponding phosphites which directly oxidized with tert-butylhydroperoxide and produced the expected diastereomeric mixtures of cycloSaligenyl products. The same preparation scheme was used to introduce the cycloSal-moiety into 3′-fluoro-3′-deoxythymidine 20 and 5-iodo-3′-fluoro-2′,3′-dideoxyuridine 21, to yield cycloSaligenyl monophosphates 23 and 24, respectively (Scheme 2). Performing phosphitylation of IUdR without protecting the 3′- or 5′-OH group on uridine, simplified the synthesis and allowed for the concurrent access to the 5′-O— and 3′-O-cycloSal-5-[¹²⁵I]IUdRMPs, and a parallel evaluation of biophysical properties in both groups of regioisomers. Typically, when phosphitylation of IUdR by chlorophosphite (1.05-1.25 molar equivalent) was carried out at a temperature below −40° C., a mixture of 5′-O— and 3′-O-cycloSaligenyl phosphotriesters was formed, with only a marginal regioselectivity. The 3′,5′-O,O-diphosphorylation occurred in the range of 16-22%. The separation of regioisomers, by flash column chromatography on silica gel, furnished pure diastereomeric 5′-O—, 3′-O— and 3′,5′-O,O-cycloSal-products in fair (39-46%) overall yield. Phosphitylations conducted using IUdR derivatives with protected 3′-OH or 5′-OH groups, did not greatly improve the overall efficiency of the synthesis due to required protection/deprotection steps. These independently synthesized regioisomers however, aided the regioisomer's elution order verification, in purifications of the reaction mixtures originated from unprotected IUdR. Thus, the 5′-O-phosphitylation of 4, along with one-pot oxidation, followed by the deprotection of 3′-O-levulinate group, led to the corresponding phosphotriesters 6-8 in 52-62% of the isolated yield.

The present inventors also examined the introduction of the cycloSal-component via the direct coupling of cyclic chlorophosphites, performed at the non-carrier-added concentration level. This approach permits a straightforward, one-step synthesis of many radiolabeled cycloSaligenyl monophosphates, starting from derivatives of deoxyuridine or thymidine already labeled with radionuclide, particularly practical for [¹⁸F]-fluorine, e.g., 3′-[¹⁸F]-fluoro-3′-deoxythymidine ([¹⁸F]FLT). 5-[¹²⁵I]-Iodo-5′-O-[cyclo-3,5-Di-(tert-butyl)-6-fluoroSaligenyl]-3′-fluoro-2′,3′-dideoxyuridine 24b was prepared through the routine [¹²⁵I]-iododestannylation of corresponding stannanes (Scheme 2) These results confirmed, that the coupling of cyclic chlorophosphites can be successfully performed at the non-carrier-added concentration level and may be fully applicable in the preparation of cycloSaligenyl-[¹⁸F]FLT derivatives, or implemented to other [¹⁸F]-radiofluorinated analogs.

The organotin precursors 6a-14a, and 24a, were acquired using hexamethylditin (except 6a wherein a tri-n-butyl derivative was used) in the reactions catalyzed by bis(triphenylphosphine)palladium(II) dichloride. Stannylations were carried out under nitrogen in boiling dioxane or ethyl acetate at 60° C., depending on the solubility of the starting iodotriester. Under these milder conditions, the proton dehalogenation was reduced from ˜20% to ˜9% at 60° C. Two major products were consistently obtained. A first product, with a shorter retention time on TLC and proven to be the trimethylstannyl derivative, was isolated in 52-77% yield. A second product (with a longer retention time on TLC) was identified as proton deiodinated starting phosphotriester. All cycloSaligenyl-5-trimethylstannyl-2′-deoxyuridine phosphotriesters synthesized in the method of this invention were amenable to no-carrier-added radio-iododestannylations providing excellent isolated yields of [¹²⁵I]-iodolabeled products. Moreover, a high hydrophobicity of synthesized stannanes in respect to corresponding iodo-derivatives, allowed for a consistent and complete separation of the trimethyltin precursor excess from the [¹²⁵]-iodo product, even if a large volume of crude reaction mixture (up to 1 mL) was loaded onto the HPLC column in final purification.

All [¹²⁵I]-radioiodinations were conducted within 0.25-12 mCi range, using acetonitrile as a solvent and ˜120 μg of the stannyl precursor. The reaction mixtures were acidified with TFA solution in acetonitrile and hydrogen peroxide was used to oxidize [¹²⁵I] sodium iodide. Modified radio-iododestannylation procedure provided a consistently excellent radiochemical yield of 85-93% and a radiochemical purity of ≧95%, across the series of cycloSaligenyl-phosphotriesters. The proton destannylated product (3-8%) was found in all of the crude reaction mixtures. The majority of it originated from frozen tin precursor samples, and increased after the prolonged storage period of the stannane (sometimes exceeding six months). In order to confirm the radiochemical purity and precisely measure the specific activity of products, the reaction mixtures were purified by HPLC, with parallel monitoring of the radioactivity and absorbance (220/280 nm). Radiolabeled compounds, if kept in a solution of aqueous acetonitrile overnight (at concentrations of ˜1 μCi/μL) were routinely re-purified just before conducting intended experiments. However, the HPLC analysis performed within 24 h generally indicated the product retained ≧95% of radiochemical purity. The HPLC co-injections of [¹²⁵I]-radiolabeled products: 6b-14b, 24b and corresponding nonradioactive iodo-analogs prepared independently, positively confirmed the identity of radioiodinated compounds. Characterization of all non-radioactive phosphotriesters was carried out by means of ¹H, ¹³C, ¹¹⁹Sn and ³¹P NMR, as well as high resolution mass spectroscopy and thorough HPLC analyses.

### Experimental Procedures

Chemicals and reagents were purchased from commercial suppliers and used without further purification. Anhydrous diethyl ether was distilled from sodium wire with benzophenone as an indicator and dichloromethane was distilled from CaH₂ under nitrogen. [¹²⁵I]NaI in 1×10⁻⁵ NaOH (pH 8-11) was obtained from PerkinElmer. Radioactivity was measured with Minaxi γ-counter (Packard, Waltham, Mass.), a dose calibrator (Cap Intec Inc., Ramsey, N.J.). Analytical TLC was carried out on precoated plastic plates, normal phase Merck 60 F₂₅₄ with a 0.2 mm layer of silica, and spots were visualized with either short wave UV or iodine vapors. Radioactive compounds on TLC and ITLC plates were analyzed on a Vista-100 plate reader (Radiomatic VISTA Model 100, Radiomatic Instruments & Chemical Co., Inc., Tampa, Fla.). Flash column chromatography was carried out using Merck silica gel 60 (40-60 μM) as stationary phase. Compounds were resolved and their purity confirmed by the HPLC analyses on Gilson (Middleton, Wis.) and ISCO (Lincoln, Nebr.) systems using 5-μm, 250×4.6 mm, analytical columns, either Columbus C8 (Phenomenex, Torrance, Calif.) or ACE C18 (Advanced Chromatography Technologies, www.ace-hplc.com). Columns were protected by guard filters and were eluted at a rate of 0.8 mL/min with various gradients of CH₃CN (10-95%) in water with or without TTA (0.07%, w/v). Variable wavelength UV detectors UVIS-205 (Linear, Irvine, Calif.) and UV116 (Gilson) were used with the sodium iodide crystal Flow-count detector (Bioscan, Washington, D.C.) connected in-line at the outlet of the UV detector. Both signals were monitored and analyzed simultaneously. NMR spectra were recorded at ambient temperature in (CD₃)₂SO or CDCl₃ with a Varian NOVA 500 MHz NMR instrument spectrometer (Palo Alto, Calif.). Chemical shifts are given as δ (ppm) relative to TMS as internal standard, with J in hertz. Deuterium exchange and decoupling experiments were performed in order to confirm proton assignments. ³¹P NMR and ¹¹⁹Sn NMR spectra were recorded with proton decoupling. High resolution (ESI-HR) positive ion mass spectra were acquired on an LTQ-Orbitrap mass spectrometer with electrospray ionization (ESI). Samples were dissolved in 70% methanol. Two μL aliquots were loaded into a 10-μL loop and injected with a 5μL/min flow of 70% acetonitrile, 0.1% formic acid. FAB high-resolution (FAB-HR) mass spectra analyses (positive ion mode, 3nitrobenzyl alcohol matrix) were performed by the Washington University Mass Spectrometry Resource (St. Louis, Mo.) and at the University of Nebraska Mass Spectrometry Center (Lincoln, Nebr.).

### General Procedure A (Synthesis of Saligenyl Chlorophosphites)

To a solution of a dried salicyl alcohol derivative in Et₂O stirred at −16° C. under a nitrogen atmosphere, newly distilled PC1₃ was added. After 15 min, while maintaining the same temperature, a solution of pyridine in Et₂O was added dropwise over a period of 1 h. The reaction mixture was allowed to reach ambient temperature and the stirring was continued for further 2 h. To facilitate a complete separation of pyridinium chloride, the mixture was stored in a tightly covered reaction flask at −20° C. overnight. After filtration under pressure of dry nitrogen, a solvent was evaporated in vacuum and the resulting crude product was used in the next step of synthesis without a delay.

### General Procedure B (Synthesis of Nonradioactive cycloSaligenyl Phosphotriesters of 5-Iodo-2′-deoxyuridine 6-14, Using Unprotected IUdR and Chlorophosphites).

All reactions were performed under anhydrous conditions and a dry nitrogen or argon atmosphere. To a stirred solution of IUdR and DIPEA (˜2.5 molar excess) in DMF/THF (2:1 mixture), cooled to or below −40° C., the THF solution of the appropriate crude chlorophosphite (1.05-1.25 molar equiv.) was added in small portions. Chlorophosphites (obtained using General Procedure A) were transferred directly from its original reaction vessel, by means of an argon pressure and the syringe equipped with a long double needle. The reaction mixture was then slowly warmed to ambient temperature and a further stirring continued for 30 min, to ensure a completion of the reaction (TLC monitoring with DCM/MeOH 10:1.0-1.2 range). The reaction mixture was cooled to −40° C. once again and a solution of tert-butyl hydroperoxide, 5-6 M (2.1-2.5 molar equiv.) in n-decane was added. The resulting mixture slowly warmed to room temperature, with the stirring continued for about 1 h (the reaction progress was followed by TLC). The solvent was evaporated under reduced pressure and the residue was treated with DCM. The precipitate of unreacted IUdR was filtered off, a precipitate washed with DCM, dried under high vacuum. The recovered IUdR was proven suitable for an immediate reuse. The filtrate was evaporated under reduced pressure and the residue purified by flash column chromatography on a silica gel, using a gradient of MeOH in DCM (0.7-1.0:10) and/or a gradient of MeOH in EtOAc (0.2-0.7:10), to yield each of three formed cycloSaligenyl regioisomers: 5′-O—, 3′-O— and di-5′,3′-O,O— substituted, separated. Several diastereomers of 5′-O-cycloSaligenyl-, as well as 3′-O— cycloSaligenyl-phosphotriesters were later separated, by means of the HPLC, giving small quantities (˜30 mg) of each individual diastereomer.

### General Procedure C (Synthesis of Nonradioactive cycloSaligenyl Phosphotriesters of 5-Iodo-2′-deoxyuridine 6-14, Thymidines and 5-Iodo-3′-dideoxy-3′-fluorouridine, Using Protected IUdR 4 or Uridines, and Chiorophosphites)

Under argon or a dry nitrogen atmosphere, DIPEA and the crude saligenyl chlorophosphite, dissolved in MeCN, were added to a stirred solution of IUdR 4 in MeCN at −40° C. The reaction mixture was slowly warmed to ambient temperature and the reaction progress monitored by the TLC (DCM/MeOH 10:0.7). The reaction mixture cooled one more time to −40° C. was treated with a solution of tent-butyl hydroperoxide 5-6 M (˜2.5 molar equiv.) in n-decane. The mixture was warmed up slowly to room temperature and stirred 1-2 h (the reaction progress was followed by TLC). The solvent was removed under reduced pressure, the residue treated with DCM (80 mL) or EtOAc (60 mL) and filtered. The filtrate was washed with the 0.3% aqueous solution of NaHSO₃ (20 mL), brine (20 mL), dried over MgSO₄ and evaporated. Deprotection Procedures: in reactions conducted with 4 the resulted solid was dissolved in pyridine (2 mL) and added to a stirred, cooled on an ice-water bath, solution of hydrazine hydrate (1.5 mL) in pyridine (3 mL), containing acetic acid (2.2 mL). The stirring continued for 5 min, and then water (40 mL) and EtOAc (50 mL) were added. The organic layer was separated and washed with the 10% aqueous solution of NaHCO₃ (20 mL), water (20 mL), dried over MgSO₄ and evaporated, and the residue was purified on a silica gel column.

### General Procedure D (Synthesis of Trialkyltin Precursors 6a-14a, 21, 24a)

The solution of appropriate iodouridine 6-14, 21 or 24 (1.0 equiv), the hexamethylditin (hexa-n-butylditin was used in the preparation of 6a) (1.25-1.50 equiv) and dichlorobis(triphenylphosphine) palladium II catalyst (0.10 equiv) in ethyl acetate or dioxane (for 6 and 7) was refluxed (2-6 h) under a nitrogen atmosphere (until the starting material disappeared). The reaction progress was monitored by TLC. Two major products were formed in all the reactions. The first product, with a higher TLC mobility, isolated in 50-72% yield, was the trialkylstannyl derivative, and a second one (with a low TLC mobility) was a proton deiodinated starting compound. After cooling to ambient temperature a mixture was freed from an excess of the catalyst and partially purified by the filtration through a thin pad of silica (EtOAc/hexanes, 2:1). The resulting crude product was purified by repeating a silica gel column chromatography, using a gradient of EtOAc in hexanes (2-5:10) and/or a gradient of MeOH in DCM or CHCl₃ (0.4-0.7:10). Anhydrous samples of pure tin precursors (˜100 μg) were stored up to eight months, with the exclusion of light, under nitrogen at −20° C.; not showing the excessive decomposition (≦7% by the HPLC analysis) and were suitable for the immediate radio-iododestannylation. Diastereomers of 5′-O-cycloSaligenyl-, as well as 3′-O-cycloSaligenyl-5-trimethyltin-phosphotriesters were later separated, by the HPLC using a reverse phase columns, to give small quantities (˜20 mg) of each, individual diastereomer.

### General Procedure E (Synthesis of [¹²⁵I]-Radioiodinated cycloSaligenyl Phosphotriesters of 5-[¹²⁵I]Iodo-2′-deoxyuridine 6b-14b, and 24b)

Into a glass tube containing the appropriate tin precursor 6a-14a, or 24a (100-120 μg, 130-150 μmol), obtained as described immediately above dissolved in MeCN (50-100 μL), a solution of Na¹²⁵I/NaOH (10-100 μL, 1-10 mCi) was added, followed by a 30% aqueous H₂O₂ solution (5 μL), and followed by TFA solution (50 μL, 0.1 N in MeCN) added with a 2 min delay. The mixture was briefly vortexed and left for 15 min at room temperature. The reaction was quenched with Na₂S₂O₃ (100 μg in 100 μL of H₂O) and taken up into a syringe. The reaction tube was washed twice with 50 μL of H₂O/MeCN (9:1) solution. The previously withdrawn reaction mixture, plus washes were injected onto the HPLC system and separated, by means of C8 or C18 reverse phase column. Eluent from a column (1 mL fractions collected) was monitored using a radioactivity detector, connected to the outlet of UV detector (detection at 220 and 280 nm). Eluted fractions containing a product, combined and evaporated with a stream of dried nitrogen, were reconstituted in an appropriate solvent and concentration, and were filtered through a sterile (Millipore 0.22 μm) filter into a sterile evacuated vial. Identity of radiolabeled products was confirmed by the evaluation of UV signals of nonradioactive iodo-analogs (prepared independently, not through the iododestannylation reaction) with the radioactive signals, and/or by comparing R_{f} obtained from the radio-TLC and t_{R} from the radio-HPLC analysis. The specific activities were determined by the UV absorbance of radioactive peaks, as compared to the standard curves of unlabeled reference compounds. Radiolabeled products, if kept in a solution overnight at ambient temperature, were repurified before conducting further experiments, even though HPLC analysis rarely indicated less than 95% of the radiochemical purity.

### Synthesis of 5-Iodo-5′-O-cycloSaligenyl-2′-deoxyuridine Monophosphate (6 and 9)

Method I: General Procedure C with 3′-O-levulinyl IUdR 4 (1.22 g, 2.7 mmol), DIPEA (1.3 mL, 0.96 g, 7.44 mmol) and crude chlorophosphite 15 (950 mg, ˜6 mmol), was conducted in 25 mL of MeCN and the oxidation carried out with a solution of t-BuOOH (0.86 mL, ≧4 mmol) after 40 min of phosphitylation. Before the deprotection of 3′-O-Lev group, a small amount of a crude product (19 mg) was purified by HPLC, on Columbus C18, 100 Å (5 μm, 10×250 mm) column, eluted at 2.5 mL/min with 37% MeCN in water. The HPLC analysis showed two diastereomers, (47:53 ratio) t_{R}=24.4 min, t_{R}=24.8 min (≧98% pure, UV at 280 nm), ACE C18, 100 Å (5 82 m, 4.6×250 mm) column; eluent: solvent A 10% MeCN in water, solvent B MeCN; eluted at 1.0 mL/min with a linear gradient of B from 0-95% over 45 min, and 95% B for 15 min. ¹H NMR (DMSO-d₆) δ=11.39 (bs, 1H, NH), 8.11, 8.08 (2 s, 1H, uridine-H6), 7.26-7.19 (m, 1H, aryl-H4), 7.12-7.06 (m, 3H, aryl-H3, aryl-H5 aryl-H6), 6.08, 6.05 (2 d, 1H, H1′, J=7.2 Hz), 5.47 (dd, 1H, H_{A}-benzyl), 5.45 (dd, 1H, H_{B}-benzyl), 5.45 (dd, 1H, H_{B}-benzyl), 5.42 (dd, 1H, H_{A}-benzyl), 5.40 (dd, 1H, H_{B}-benzyl), 5.17-5.09 (m, 1H, H5″), 4.44-4.39 (m, 2H, H3′, H5′), 4.19-4.13 (m, 1H, H4′), 3.33-3.29 (m, 2H, Lev-C3-CH₂), 2.76-2.74 (m, 2H, Lev-C2-CH₂), 2.40-2.23 (m, 1H, H2″), 2.22, 2.20 (2 s, 3H, Lev-C5-CH₃), 2.21-2.16 (m, 1H, H2′) ppm. ³¹P NMR (DMSO-d₆) δ=−8.29 −8.32 (2 s, diastereomeric mixture) ppm. The deprotection of 3′-O-Lev group was completed in less then 10 min (a single band on TLC) and the crude product was purified on a silica gel column DCM/MeO gradient, 10:0.7-1.0), to give 6 (735 mg, 52%) as colorless foam; R_{f} value 0.42 (DCM/MeOH, 10:0.7). HPLC analysis has shown a diastereomeric mixture (4:5 ratio): t_{R}=20.2 and t_{R}=22.2 min (˜98% pure, UV at 280 nm), conducted on Jupiter C18 300 Å (5 μm, 4.6×250 mm) column; eluent: solvent A 20% MeCN in water, solvent B MeCN; eluted at 0.8 mL/min with A for 25 min, then a linear gradient of B from 0-95% over 10 min, and finally 95% B for 10 min. ¹H NMR (DMSO-d₆) δ=11.68, 11.63 (2 s, 1H, NH), 7.89, 7.78 (2 s, 1H, uridine-H6), 7.33 (tt, 1H, aryl-H4, J=7.5 Hz), 7.25-7.19 (m, 2H, aryl-H6, aryl-H5), 7.17-7.12 (m, 1H, aryl-H3), 6.03, 5.98 (2 t, 1H, 1′, ³J_{H,H}=7.0 Hz), 5.22-5.19 (m, 2H, benzyl), 5.42 (d, 1H, C3′-OH, J=4.6 Hz), 4.36-4.23 (m, 2H, H5″, H5′), 4.22-4.18 (m, 1H, H3′), 3.94-3.89 (m, 1H, H4′), 2.23-2.12 (m, 1H, H2″), 2.10-2.05 (m, 1H, H2′) ppm. ³¹P NMR (DMSO-d₆) δ=−9.18, −9.33 (2 s, diastereomeric mixture) ppm. MSFAB-HR (m/z): [M+Li]⁺calcd for C₁₆H₁₆N₂O₈PILi, 528.9849, found 528.9837. Diastereomers of 6 were separated by preparative HPLC on Columbus C18, 100 Å (5 μm, 10×250 mm) column, eluted at 2.6 mL/min with 20% MeCN in water. The amount of 59 mg of the diastereomeric mixture used for the separation, gave 19.1 mg of 6 fast and 17.2 mg of 6 slow. Analytical data of diastereomer 6 fast are as follows: ¹H NMR (DMSO-d₆) δ=11.69 (s, 1H, NH), 7.98 (s, 1H, uridine-H6), 7.35 (tt, 1H, aryl-H4, ³J_{HH}=7.8 Hz, ⁴J_{HH}=1.0 Hz), 7.27, 7.25 (2 d, 1H, aryl-H6, J=1.5 Hz), 7.18 (tt, 1H, aryl-H5, ³J_{HH}=7.5 Hz, ⁴J_{HH}=1.0 Hz), 7.14, 7.12 (2 d, 1H, aryl-H3, ³J_{HH}=7.6 Hz, ⁴J_{HH}=1,0 Hz), 6.07 (dd, 1H, H1′, J_{1′-2″}=7.4 Hz, J_{1′-2″}=6.5 Hz), 5.55-5.44 (m, 2H, benzyl), 5.41 (d, 1H, C3′-OH, J=4.4 Hz), 4.39 (dd, 1H, H5″, ²J_{HH}=11.3 Hz, ³J_{HH}=7.1 Hz, ³J_{HP}=3.7 Hz), 4.29 (dd, 1H, H5′, ²J_{HH}=11.6 Hz, ³J_{HH}=6.7 Hz, ³J_{HP}=4.6 Hz), 4.18-4.16 (m, 1H, H3′), 3.94-3.91 (m, 1HH, H4′), 2.19-2.16 (m, 1H, H2″), 2.09-2.05 (m, 1H, H2′) ppm. ¹³C NMR (DMSO-d₆) δ=160.77 (C4), 150.38 (C2), 149.35 (C2-aryl), 144.33 (C6), 129.62 (C4-aryl), 126.0 (C6-aryl), 124.31 (C5-aryl), 120.93 (C1-aryl), 118.0 (C3-aryl), 109.42 (C5), 84.75 (C1′), 84.32 (C4′), 69.92 (C3′), 68.86 (C-benzyl), 67.64 (C5′), 38.72 (C2′) ppm. ³¹P NMR (DMSO-d₆) δ=−9.19 ppm. Analytical data of diastereomer 6 slow are as follows: ¹H NMR (DMSO-d₆) δ=11.71 (s, 1H, NH), 7.99 (s, 1H, uridine-H6), 7.37 (tt, 1H, aryl-H4, ³J_{HH}=7.7 Hz, ⁴J_{HH}=1.1 Hz), 7.28 (dd, 1H, aryl-H6, J=1.5 Hz), 7.20 (tt, 1H, aryl-H5, ³J_{HH}=7.6 Hz, ⁴J_{HH}=1.0 Hz), 7.15 (dd, 1H, aryl-H3, ³J_{HH}=7.6 Hz, ⁴J_{HH}=1,0 Hz), 6.10 (dd, 1H, H1′, J_{1′-2″}=7.5 Hz, J_{1′-2″}=6.4 Hz), 5.53-5.45 (m, 2H, benzyl), 5.43 (d, 1H, C3′-OH, J=4.5 Hz), 4.35-4.27 (m, 2H, H5″ and H5′), 4.22-4.18 (m, 1H, H3′), 3.94-3.90 (m, 1H, H4′), 2.22-2.17 (m, 1H, H2″), 2.10-2.05 (m, 1H, H2′) ppm. ¹³C NMR (DMSO-d₆) δ=160.81 (C4), 150.42 (C2), 149.39 (C2-aryl), 144.41 (C6), 129.59 (C4-aryl), 126.12 (C6-aryl), 124.29 (C5-aryl), 120.87 (C1-aryl), 118.90 (C3-aryl), 110.02 (C5), 84.83 (C1′), 84.29 (C4′), 70.13 (C3′), 68.90 (C-benzyl), 67.76 (C5′), 38.59 (C2′) ppm. ³¹P NMR (DMSO-d₆) δ=−9.32 ppm.

Method II: General Procedure B conducted with IUdR (1.12 g, 3.16 mmol), dissolved in 15 mL of DMF, DIPEA (1.14 mL, 0.84 g, 6.5 mmol), and newly distilled chlorophosphite 15 (604 mg, 3.83 mmol) diluted with 6 mL of dry THF and transferred in three (2 mL) portions; the oxidation with a solution of t-BuOOH (0.86 mL, ≧4.3 mmol). The time of phosphitylation 2 h, the oxidation was carried out for 1 h. All three phosphotriesters purified on a silica gel column (DCM/MeOH gradient, 10:0.7-1.0), were collected in a form of colorless foam: 5′,3′-O,O′-dicycloSal-5-IUdRMP 9 (R_{f} 0.72), 350 mg (16%); 3′-O-cycloSal-IUdRMP 12 (R_{f} 0.58), 611 mg (37%); 5′-O-cycloSal-5-IUdRMP 6 (R_{f}0.42), 759 mg (46%). The analytical data of product 6 were identical with those reported above for 6 obtained in Method I.

### Synthesis of 5-Iodo-5′-O-cyclo(3-methylSaligenyl)-2′-deoxyuridine Monophosphate (7 and 10)

Method I: General Procedure C with 3′-O-levulinyl IUdR 4 (1.36 g, 3.0 mmol), DIPEA (1.5 mL, 1.11 g, 8.6 mmol) and the crude chlorophosphite 16 (1.04 g, ˜6 mmol), was conducted in 27 mL of dry MeCN. The reaction time was extended to 4 h (TLC monitoring), the oxidation proceeded with a solution of t-BuOOH (0.9 mL, ≧4.5 mmol). Before the deprotection of 3′-O-Lev group, a small sample of a crude product (˜11 mg) was purified by HPLC, on Columbus C18, 100 Å (5 μm, 10×250 mm) column, eluted at 2.5 mL/min with 40% MeCN in water. The HPLC analysis showed two diastereomers, (45:55 ratio) t_{R}=26.3 min, t_{R}=26.6 min (≧98% pure, UV at 280 nm), ACE C18, 100 Å (5 μm, 4.6×250 mm); eluent: solvent A 10% MeCN in water, solvent B MeCN; eluted at 1.0 mL/min with a linear gradient of B from 0-95% over 45 min, and 95% B for 15 min. Purified 3′-O-Lev derivative of 7, was further analyzed: ¹H NMR (DMSO-d₆) δ=11.77 (bs, 1H, NH), 8.08, 8.05 (2 s, 1H, uridine-H6), 7.26-7.14 (m, 1H, aryl-H5), 7.10-7.08 (m, 2H, aryl-H6, aryl-H4), 6.09 (dd, 1H, H1′, J_{1′-2′}=7.3 Hz, J_{1′-2″}=6.4 Hz), 5.53-5.42 (m, 2H, benzyl), 5.15-5.09 (m, 1H, H3′), 4.41-4.36 (m, 2H, H4′, H5″), 4.15-4.11 (m, 1H, H5′), 3.35-3.33 (m, 2H, Lev-C3-CH₂), 2.74-2.72 (m, 2H, Lev-C2-CH₂), 2.41-2.24 (m, 4H, H2″, Lev-C5-CH₃), 2.23-2.18 (m, 1H, H2′), 2.11 (s, 3H, aryl-C3-CH3) ppm. ³¹P NMR (DMSO-d₆) δ=−8.23, −8.27 (2 s, diastereomeric mixture) ppm. MSFAB-HR (m/z): [M+H]⁺calcd for C₂₂H₂₅N₂O₁₀PI, 635.0292, found 635.0277. Subsequent cleavage of 3′-O-Lev group completed in <5 min and the crude product was purified on a silica gel column (DCM/MeOH, 10:0.7), to give 7 (933 mg, 58%) as colorless foam; R_{f} value 0.46 (DCM/MeOH, 10:0.7). The HPLC analysis showed a mixture of diastereomers: t_{R}=18.2 min, t_{R}=18.8 min (≧98% pure, UV at 280 nm), ACE C18, 100 Å (5 μm, 4.6×250 mm) column; eluent: solvent A 10% MeCN in water, solvent B MeCN; eluted at 1.0 mL/min with a linear gradient of B from 0-95% over 45 min, and 95% B for 15 min. ¹H NMR (DMSO-d₆) δ=11.73, 11.68 (2 s, 1H, NH), 7.99, 7.86 (2 s, 1H, uridine-H6), 7.25-7.21 (m, 1H, aryl-H5), 7.12-7.07 (m, 2H, aryl-H6, aryl-H4), 6.08, 6.05 (dd, 1H, H1′), 5.51-5.39 (m, 2H, benzyl), 5.41 (d, 1H, C3′-OH′, J=3.7 Hz), 4.34-4.26 (m, 1H, H5″, H3′), 4.22-4.18 (m, 1H, H5′), 3.93-3.89 (m, 1H, H4′), 2.25-2.19 (m, 4H, H2″,), 2.23 (s, 3H, C3-aryl-CH₃), 2.10-2.05 (m, 1H, H2″) ppm. ¹³C NMR (DMSO-d₆) δ=162.87 (C4), 150.31 (C2), 148.55, 148.51 (C2-aryl), 141.23, 141.18 (C6), 132.86 (C5-aryl), 129.18 (C4-aryl), 126.33 (C6-aryl), 120.73, 120.66 (C1-aryl), 117.35 (C3-aryl), 109.67 (C5), 84.75, 84.65 (C1′), 84.32, 84.22 (C4′), 69.98 (C3′), 68.82 (C-benzyl), 67.64 (C5′), 38.72 (C2′), 21.11 (CH₃-aryl) ppm.³¹P NMR (DMSO-d₆) δ=−8.89, −8.93 (2 s, diastereomeric mixture) ppm. MSFAB-HR (m/z): [M+H]⁺calcd for C₁₆H₁₉N₂O₈PI, 536.9924, found 536.9899. Diastereomers were separated by preparative HPLC on Columbus C18, 100 Å (5 μm, 10×250 mm) column; eluted with 22% MeCN in water at 2.5 mL/min flow rate. From the total of 66 mg diastereomeric 7 used for separation, 14.1 mg of 7 fast and 12.2 mg of 7 slow was isolated. Diastereomer 7 fast eluted within 72-76 min, and 7 slow within 79-81 min after the injection, and each isomer was collected in ˜9 mL of eluent. A solvent was evaporated to dryness in high vacuum; the product residue was reconstituted in MeCN and analyzed one more time on the analytical HPLC. Each diastereomer was found to be ≧97% pure (UV at 220 and 280 nm). Analytical data of 7 fast are as follows:¹H NMR (DMSO-d₆) δ=11.70 (s, 1H, NH), 7.98 (s, 1H, uridine-H6), 7.23-7.20 (m, 1H, aryl-H5), 7.17 (dd, 1H, aryl-H4, J=1.5 Hz), 7.12-7.06 (m, 1H, aryl-H6), 6.06 (dd, 1H, H1′, J=6.6 Hz), 5.51-5.43 (m, 3H, benzyl, C3′-OH), 4.38-4.33 (m, 1H, H5″), 4.27-4.22 (m, 1H, H5′), 4.17-4.15 (m, 1H, H3′), 3.92-3.90 (m, 1H, H4′), 2.21-2.19 (m, 3H, C3-aryl-CH₃), 2.18-2.13 (m, 1H, H2″), 2.10-2.05 (m, 1H, H2′) ppm. ³¹P NMR (DMSO-d₆) δ=−8.89 ppm. Analytical data of 7 slow are as follows: ¹HNMR (DMSO-d₆) δ=11.69 (s, 1H, NH), 7.88 (s, 1H, uridine-H6), 7.24 (dd, 1H, aryl-H5), 7.09-7.06 (m, 2H, aryl-H6, aryl-H4), 6.07 (dd, 1H, H1′, J=6.5 Hz), 5.49-5.38 (m, 3H, benzyl, C3′-OH), 4.33-4.25 (m, 2H, H5″, H5′), 4.21-4.17 (m, 1H, H3′), 3.92-3.89 (m, 1H, H4′), 2.24-2.18 (m, 4H, H2″, C3-aryl-CH₃), 2.11-2.06 (m, 1H, H2′) ppm. ³¹P NMR (DMSO-d₆) δ=−8.94 ppm.

Method II: General Procedure B was conducted with IUdR (2.51 g, 7.09 mmol) dissolved in 18 mL of DMF and DIPEA (1.54 mL, 1.14 g, 8.8 mmol). The crude chlorophosphite 16 (1.39 g, ˜8 mmol) was dissolved in 6 mL of dry THF transferred in 3×2 mL portions. A solution of t-BuOOH (1.65 mL, ≧8.25 mmol) was added after 3 h of phosphitylation. The oxidation was carried out for 2 h. Phosphotriesters were purified on a silica gel column (DCM/MeOH gradient, 10:0.7-1.0) and followed by a second purification (DCM/MeOH, 10:0.4), to achieve a complete separation of the closely eluting 3′-O-isomer 13. All three products: 5′,3′-O,O′-dicyc/oSal-5-IUdRMP 10 (R_{f} 0.77), 1.12 g (22%); 3′-O-cycloSal-IUdRMP 13 (R_{f} 0.64), 1.17 g (31%); 5′-O-cycloSal-5-IUdRMP 7 (R_{f}0.47), 1.47 g (39%) were obtained as colorless foams. The analytical data of product 7 were identical with those reported above for 7 obtained according to Method I.

### Synthesis of 5-Iodo-5′-O-[cyclo-3,5-di-(tert-butyl)-6-fluoroSaligenyl]-2′-deoxyuridine Monophosphate (8 and 11)

Method I: General Procedure C was carried out with 3′-O-levulinyl IUdR 4 (1.04 g, 2.3 mmol), DIPEA (1.25 mL, 0.93 g, 7.15 mmol) and a crude chlorophosphite 17 (1.14 g, ˜3.6 mmol) in MeCN (20 mL). The oxidation with a solution of t-BuOOH (1.0 mL, ≧5 mmol) was started after 1 h of phosphitylation. A small portion (˜14 mg) of the crude product was purified by HPLC, on Columbus C18, 100 Å (5 μm, 10×250 mm) column, eluted at 3.0 mL/min with 43% MeCN in water. The purified 3′-O-Lev derivative of 8 (˜7 mg), was further analyzed by HR-MS: MSFAB-HR (m/z): [M+Li]⁺calcd for C₂₉H₃₇N₂O₁₀PIFLi, 757.1375, found 757.1355. The ¹³C peak was observed at 758.1393, within −2.0 ppm of the expected value of 758.1409. The HPLC analysis showed a mixture of two diastereomers: t_{R}=22.2 min, t_{R}=22.7 min (≧98% pure, UV at 280 nm), performed on ACE C18, 100 Å (5 μm, 4.6×250 mm) column; eluent: solvent A 50% MeCN in water, solvent B MeCN; eluted at 1.0 mL/min with a linear gradient of B from 0-55% over 25 min, then 55-95% B for the period of 20 min and 95% B for 15 min. Cleavage of 3′-O-Lev group was completed in <10 min (TLC monitoring) and the crude product was purified on a silica gel column (DCM/MeOH gradient, 10:0.7-0.9), to give 8 (930 mg, 62%) in a form of colorless foam; R_{f} value 0.52 (DCM/MeOH, 10:0.7). The HPLC analysis confirmed again a presence of two diastereomers: t_{R}=20.9 min and t_{R}=22.8 min (≧98% pure, UV at 220 and 280 nm). The analysis was conducted using Columbus C8, 100 Å (5 μm, 4.6×250 mm) column; eluent: solvent A 50% MeCN in water, solvent B MeCN; a column eluted at 1.0 mL/min with A for 25 min, then a linear gradient of B from 0-95% over 15 min, and 95% B for the period of 20 min.¹H NMR (DMSO-d₆) δ=11.11, 11.04 (2 s, 1H, NH), 7.94, 7.92 (2 s, 1H, uridine-H6), 7.28-7.25 (m, 1H, aryl-H4), 6.25-6.20 (m, 1H, H1′), 5.52-5.38 (m, 2H, benzyl), 4.64-4.57 (m, 2H, C3′-OH, H3′), 4.37-4.24 (m, 1H, H5″, H5′), 4.17-4.14 (m, 1H, H3′), 2.52-2.45 (m, 1H, H4′), 2.25-2.20 (m, 1H, H2″), 2.18-2.12 (m, 1H, H2′), 1.41 (s, 9H, 3×CH₃-t-Bu), 1.37 (s, 9H, 3×CH₃-t-Bu) ppm.¹³C NMR (DMSO-d6) δ=163.27 (C4), 155.13 (C6-aryl), 150.91 (C2), 148.51 (C2-aryl), 141.18 (C6), 133.46 (C5-aryl), 130.38 (d, C3-aryl, ⁴J_{C,F}=4.1 Hz), 127.15 (C4-aryl), 110.66 (C1-aryl), 109.77 (C5), 89.65 (C1′), 84.56, 84.45 (C4′), 71.06 (C3′), 69.22 (C-benzyl), 67.55 (C5′), 38.67 (C2′), 34.71 (1×C-tBu), 34.60 (1×C-tBu), 34.38 (d, 2×C-tBu, ⁴J_{C,F}=3.2 Hz), 29.91 (d, 2×CH₃-tBu, ⁴J_{C,F}=3.2 Hz), 29.86 (1×CH₃-tBu), 29.81 (1×CH₃-tBu) ppm. ³¹P NMR (DMSO-d₆) δ=−8.67, −8.93 (2 s diastereomeric mixture) ppm. MSFAB-HR (m/z): [M+H]⁺calcd for C₂₄H₃₂N₂O₈PFI, 653.0925, found 653.0930. The ¹³C peak was measured at 654.0945, within −2.0 ppm of the expected value.

Diastereomers were isolated by preparative HPLC (≦6 mg of 8 per injection), on Columbus C18, 100 Å (5 μm, 10×250 mm) column; eluted at 2.5 mL/min with 47% MeCN solution in water. The separation started with 96 mg of the diastereomeric 8 and 34.4 mg of 8 fast and 43.2 mg of 8 slow was isolated. Diastereomer 8 fast eluted within 26-27.5 min, and 8 slow within 28.5-30.5 min after the injection, and each isomer was collected in ˜5 mL of eluent. Solvent was evaporated to dryness in high vacuum; the product residue was reconstituted in MeCN and analyzed again on the analytical HPLC, showing the purity of ≧98% for each diastereomer. Analytical data of 8 fast are as follows:¹HNMR (DMSO-d6) δ=11.70 (s, 1H, NH), 7.98 (s, 1 H, uridine-H6), 7.22 (d, 1H, aryl-H4, ⁴J_{HF}=9.4 Hz), 6.06 (dd, 1H, H1′, J=6.6 Hz), 5.51-5.43 (m, 2H, benzyl), 5.42 (bd, 1H, C3′-OH, J=3.6 Hz), 4.38-4.33 (m, 1H, H3′), 4.27-4.22 (m, 1H, H4′), 4.17-4.15 (m, 1H, H5′), 3.92-3.90 (m, 1H, H5″), 2.21 (s, 3H, aryl C5-CH₃), 2.18-2.13 (m, 1H, H2′), 2.10-2.05 (m, 1H, H2″), 1.43 (s, 9H, 3×CH₃-t-Bu), 1.35 (s, 9H, 3×CH₃ t-Bu) ppm. ³¹P NMR (DMSO-d₆) δ=−8.89 ppm. Analytical data of 8 slow are as follows: ¹HNMR (DMSO-d₆) δ=11.69 (s, 1H, NH), 7.99 (s, 1H, uridine-H6), 7.25-7.22 (m, 1H, aryl-H5), 7.09-7.06 (m, 2H, aryl-H3, aryl-H4), 6.07 (dd, 1H, H1′, J=6.5 Hz), 5.49-5.38 (m, 3H, benzyl, C3′-OH), 4.33-4.25 (m, 2H, H3′, H4′), 4.21-4.17 (m, 1H, H5′), 3.92-3.89 (m, 1H, H5″), 2.24-2.18 (m, 4H, H2′, aryl C5-CH₃), 2.18-2.13 (m, 1H, H2′), 2.10-2.06 (m, 1H, H2″), 1.37 (s, 9H, 3×CH₃-t-Bu), 1.34 (s, 9H, 3×CH₃-t-Bu) ppm. ³¹P NMR (DMSO-d₆) δ=−8.93 ppm.

Method II: General Procedure B was conducted with IUdR (5.0 g, 14.1 mmol) in 30 mL of DMF and DIPEA (3.1 mL, 2.29 g, 17.7 mmol), crude chlorophosphite 17 (5.4 g, ˜17 mmol, dissolved in 10 mL of dry THF), transferred slowly in 2×5 mL portions. The oxidation with a solution of t-BuOOH (4 mL, ≧20 mmol) was carried out for 1 h. Phosphotriesters of IUdR were purified on a silica gel column (DCM/MeO gradient, 10:0.7-0.9). All three products were obtained in a form of colorless rigid foam: 5′,3′-O,O′-dicyc/oSal-5-IUdRMP 11 (R_{f} 0.81), 2.81 g (21%); 3′-O-cycloSal-IUdRMP 14 (R_{f} 0.66), 3.31 g (36%); 5′-O-cycloSal-5-IUdRMP 8 (R_{f} 0.52), 3.95 g (43%). The analytical data of product 8 were identical with those reported above for 8 obtained using Method I.

### Synthesis of 5′-O-[cyclo-3,5-Di-(tert-butyl)-6-fluoroSaligenyl]-3′-deoxy-3′-fluorothymidine Monophosphate (23)

General Procedure C with 3′-deoxy-3′-fluorothymidine 20 (454 mg, 1.86 mmol) in DMF (3 mL), DIPEA (820 μL, 0.61 g, 4.7 mmol) and crude chlorophosphite 17 (0.65 g, ˜2 mmol, 2 mL of THF solution) was conducted for 1 h and the phosphitylated mixture oxidized with a solution of t-BuOOH (400 μL, ≧2 mmol). The crude product was separated on a silica gel column (DCM/MeOH gradient, 10:0.7-0.9) to give 23 (806 mg, 80%) as colorless rigid foam; R_{f} value 0.69 (DCM/MeOH, 10:0.7). The HPLC analysis showed a mixture of diastereomers: 23 fast, t_{R}=45.4 min and 23 slow, t_{R}=47.0 min (≧97% pure, UV at 280 nm); column: ACE C18, 100 Å (5 μm, 4.6×250 mm); eluent: solvent A 50% MeCN in water, solvent B MeCN; eluted at 0.8 mL/min with solvent A for 60 min (isocratic) and then a linear gradient of B from 0-95% B over 30 min. ¹HNMR (DMSO-d₆) δ=11.31 (s, 1H, NH), 7.47 (s, 1H, uridine-H6), 7.43 (s, 1H, uridine-H6), 7.25 (s, 114, aryl-H4), 7.23 (s, 1H, aryl-H4), 6.22-6.16 (m, 1H, H1′), 5.59-5.46 (m, 3H, benzyl, H3′), 4.43-4.39 (m, 2H, H5′, H5″), 3.95-3.91 (m, 1H, H4′), 2.17-2.10 (m, 2H, H2′, H2″), 1.71 (s, 3H, uridine-C5-CH₃), 1.66 (s, 3H, uridine-C5-CH₃), 1.34, 1.31 (overlapped s, 18H, aryl-C3-3×CH₃-tBu and aryl-C5-3×CH₃-tBu) ppm. ³¹P NMR (DMSO-d₆) δ=−9.46, −9.62 ppm. MSFAB-HR (m/z): [M+H]⁺calcd for C₂₅H₃₅N₂O₇PF₂, 544.2149, found 544.2152. The ¹³C isotope peak measured 545.2183; −2.0 ppm of the expected value.

### Synthesis of 5-Iodo-5′-O-[cyclo-3,5-Di-(tert-butyl)-6-fluoroSaligenyl]-3′-fluoro-2′,3′-dideoxyuridine Monophosphate (24)

General Procedure C with 5-iodo-3′-fluoro-2′,3′-dideoxyuridine 21 (390 mg, 1.1 mmol) in DMF (3 mL), DIPEA (480 μL, 355 mg, 2.75 mmol) and crude chlorophosphite 17 (1.95 g, ˜6 mmol, 4.4 mL of THF solution) was conducted for 45 min. Phosphitylation was followed by the oxidation with a solution of t-BuOOH (650 μL, ≧3.25 mmol). The crude product was separated on a silica gel column (DCM/MeOH gradient, 10:0.6-0.9) to give 24 (181 mg, 76%) as colorless rigid foam; R_{f} value 0.73 (DCM/MeOH, 10:0.7). The HPLC analysis showed a mixture of diastereomers: 24 fast, t_{R}=25.9 min and 24 slow, t_{R}=26.7 min (≧95% pure, UV at 280 nm); column: ACE C18, 100 Å (5 μm, 4.6×250 mm); eluent: solvent A 50% MeCN in water, solvent B MeCN; the column eluted with a linear gradient of B from 0-95% B over 60 min, and 95% B for 30 min at 1 mL/min. ¹HNMR (DMSO-d₆) δ=11.78 (s, 1H, NH), 8.12 (s, 1H, uridine-H6), 8.08 (s, 1H, uridine-H6), 7.25 (s, 1H, aryl-H4), 7.24 (m, 1H, aryl-H4), 6.14-6.10 (m, 1H, H1′), 5.57-5.46 (m, 2H, benzyl), 5.37-5.25 (m, 1H, H3′), 4.51-4.32 (m, 3H, H4′, H5′, H5″), 2.51-2.43 (m, 2H, H2′, H2″), 1.35, 1.34, 1.33, 1.32 (overlapped s, 18H, aryl-C3-3×CH₃ tBu and aryl-C5-3×CH₃-tBu) ppm. ³¹P NMR (DMSO-d₆) δ=−9.35, −9.79 ppm. MSFAB-HR (m/z): [M+Li]⁺calcd for C₂₄H₃₀N₂O₇PF₂ILi, 661.0964, found 661.0953 The ¹³C peak measured 662.0986, which was within −1.6 ppm of the expected value of 662.0998. Diastereomers were separated by preparative HPLC (≦4 mg of 24 per injection), on Columbus C18, 100 Å (5 μm, 10×250 mm) column, eluted at 2.2 mL/min with 57% MeCN in water. Starting with 61 mg of diastereomeric 24, 14.1 mg of 24 fast and 16.0 mg of 24 slow was isolated. Diastereomer 24 fast eluted within 37-39 min, and 24 slow within 39.5-41.3 min after the injection. The HPLC analysis: 24 fast, t_{R}=37.7 min and 24 slow, t_{R}=40.2 min (each isomer was ≧98% pure, UV at 280 nm); column: ACE C18, 100 Å (5 μm, 4.6×250 mm); eluent: solvent A 50% MeCN in water, solvent B MeCN; eluted with a linear gradient of B from 0-10% B over 60 min at 0.8 mL/min. Analytical data of 24 fast are as follows:¹HNMR (DMSO-d₆) δ=11.76 (s, 1H, NH), 8.14 (s, 1H, uridine-H6), 7.27 (s, 1H, aryl-H4), 6.14-6.10 (m, 1H, H1′), 5.57-5.49 (m, 2H, benzyl), 5.37-5.29 (m, 1H, H3′), 4.53-4.41 (m, 3H, H4′, H5′, H5″), 2.53-2.45 (m, 2H, H2′, H2″), 1.36, 1.32 (overlapped s, 18H, aryl-C3-3×CH₃-tBu and aryl-C5-3×CH₃-tBu) ppm. ³¹P NMR (DMSO-d₆) δ=−9.36 ppm. Analytical data of 24 slow are as follows: ¹HNMR (DMSO-d₆) δ=11.72 (s, 1H, NH), 8.08 (s, 1H, uridine-H6), 7.23 (m, 1H, aryl-H4), 6.12-6.09 (m, 1H, H1′), 5.53-5.41 (m, 2H, benzyl), 5.34-5.23 (m, 1H, H3′), 4.55-4.44 (m, 2H, H4′, H5′), 3.98-3.94 (m, 1H, H5″), 2.51-2.41 (m, 2H, H2′, H2″), 1.33, 1.31 (overlapped s, 18H, aryl-C3-3×CH₃-tBu and aryl-C5-3×CH₃-tBu) ppm. ³¹P NMR (DMSO-d₆) δ=−9.77 ppm.

### Synthesis of 5-Tri-n-butylstannyl-5′-O-cycloSaligenyl-2′-deoxyuridine Monophosphate (6a)

General Procedure D was conducted with 5-iodo-5′-O-cycloSaligenyl-2′-deoxyuridine monophosphate 6 (520 mg, 1.0 mmol), hexa-n-butylditin (750 μL, 868 mg, 1.5 mmol) and the palladium(II) catalyst (77 mg, 0.11 mmol) in dioxane (35 mL) for 70 min. The crude product was purified by column chromatography on a silica gel, using a gradient of EtOAc in hexanes (2-7:10). Further drying, by the repeated evaporation with anhydrous MeCN and the exposure to high vacuum, gave 6a (360 mg, 52%) as rigid colorless foam; R_{f} value 0.69 (EtOAc/hexanes, 2:1). The HPLC analysis showed a mixture of diastereomers 6a fast, t_{R}=73.9 min and 6a slow, t_{R}=74.4 min (≧97% pure, UV at 220 and 280 nm); column: ACE C18, 100 Å (5 μm, 4.6×250 mm); eluent: solvent A water, solvent B MeCN; eluted at 0.8 mL/min with a linear gradient of B from 10-50% over 30 min, then 50% B (isocratic) for the period of 30 min, and a linear gradient of B from 50-95% for 30 min. Diastereomers were not separated on a preparative scale.¹HNMR (CDCl₃) δ=8.32 (s, 1H, NH), 8.27 (s, 1H NH), 7.33 (s, 1H, uridine-H6), 7.31 (s, 1H, uridine-H6), 7.15 (t, 1H, aryl-H5, J=7.6 Hz), 7.13-7.06 (m, 3H, aryl-H3, H4, H6), 6.15-6.12 (m, 1H, H-1′), 5.39-5.34 (m, 2H, benzyl),4.58-4.54 (m, 1H, H3′), 4.45-4.37 (m, 2H, H5′, H5″), 4.12-4.09 (m, 1H, H4′), 3.23 (bd, 1H, C3′-OH, exchangeable with D₂O, J=4.5 Hz), 2.46-2.40 (m, 1H, H2′), 2.35-2.25 (m, 1H, H2″),1.56-1.42 (m, 3×2H, 3×CH₂-n-Bu), 1.35-1.26 (m, 3×2H, 3×CH₂-n-Bu), 1.02-0.98 (m, 3×2H, 3×CH₂-n-Bu), 0.92-0.87 (m, 3×3H, 3×3×CH₃-n-Bu) ppm. ³¹P NMR (CDCl₃) δ=−8.23, −8.08 ppm. ¹¹⁹Sn NMR (CDCl₃) δ=−1.97 ppm. MSFAB-HR (m/z): [M+Li]⁺calcd for C₂₈H₄₃N₂O₈PSnLi, 693.1939, found 693.1943.

### Synthesis of 5-Trimethylstannyl-5′-O-cyclo(3-methylSaligenyl)-2′-deoxyuridine Monophosphate (7a)

General Procedure D was conducted with 5-iodo-5′-O-cyclo(3-methylsaligenyl)-2′-deoxyuridine monophosphate 7 (710 mg, 1.32 mmol), hexamethylditin (0.64 g, 1.95 mmol) and the palladium(II) catalyst (90 mg, 0.13 mmol) in dioxane (40 mL) for 2 h. Purification of the crude product on a silica gel column (CHCl₃/MeOH, 10:0.8) and the repeated twice evaporation from dried MeCN, gave pure stannane 7a (374 mg, 49%) as colorless foam; R_{f} value 0.67 (DCM/MeOH, 10:0.8). Purified 7a was analyzed on HPLC, using ACE C18, 100 Å (5 μm, 4.6×250 mm) column; eluent: solvent A 10% MeCN in water, solvent B MeCN; eluted at 1 mL/min with a linear gradient of B from 0-70% over 90 min. The analysis showed a mixture of diastereomers 7a fast t_{R}=55.2 min, 7a slow t_{R}=55.4 min (≧98% pure, UV at 280 nm). Diastereomers were not separated preparatively. ¹HNMR (CDCl₃) δ=8.13 (s, 1H, NH), 8.11 (s, 1H NH), 7.33 (s, 1H, uridine-H6, ³J_{H,Sn}=18.4 Hz), 7.29 (s, 1H, uridine-H6, ³J_{H,Sn}=18.4 Hz), 7.17 (m, 1H, aryl-H5), 7.12-7.04 (m, 3H, aryl-H3, aryl-H4, aryl-H6), 6.20-6.15 (m, 1H, H1′), 5.41-5.34 (m, 2H, benzyl), 4.55-4.52 (m, 1H, H3′), 4.47-4.39 (m, 2H, H4′, H5′), 4.16-4.11 (m, 1H, H5″), 3.57 (bd, 1H, C3′-OH, exchangeable with D₂O), 2.45-2.36 (m, 1H, H2′), 2.34-2.27 (m, 1H, H2″), 2.21-2.19 (m, 3H, aryl-C3-CH₃), 0.49 (s, 9H, 3×CH₃, ²J_{Sn,H}=29.2 Hz) ppm.³¹P NMR (CDCl₃) δ=−8.37, −8.18 ppm. ¹¹⁹Sn NMR (CDCl₃) δ=−1.07 ppm. MSFAB-HR (m/z): [M+H]⁺calcd for C₂₀H₂₈N₂O₈PSn, 575.0605, found 575.0593. For the ¹³C peak measured 576.0640; −0.2 ppm.

### Synthesis of 5-Trimethylstannyl-5′-O-[cyclo-3,5-di-(tert-butyl)-6-fluoroSaligenyl]-2′-deoxyuridine Monophosphate (8a)

General Procedure D was conducted with 5-iodo-5′-O-[cyclo-3,5-di-(tert-butyl)-6-fluorosaligenyl]-2′-deoxyuridine monophosphate 8 (1.51 g, 2.3 mmol), hexamethylditin (0.98 g, 3.0 mmol) and the palladium(II) catalyst (160 mg, 0.22 mmol) in EtOAc (80 mL) for 2 h. Purification of the crude product required two silica gel columns: DCM/MeOH, 10:0.7 and EtOAc/MeOH gradient, 10:0-0.2, and a final evaporation from dried MeCN, to give a pure stannane 8a (1.22 g, 76%) as colorless foam; R_{f} value 0.77 (DCM/MeOH, 10:0.7). The HPLC analysis showed a mixture of diastereomers: 8a fast, t_{R}=43.7 min and 8a slow, t_{R}=44.1 min (≧98% pure, UV at 220 and 280 nm); column: ACE C18, 100 Å (5 μm, 4.6 ×250 mm); eluent: solvent A 50% MeCN in water, solvent B MeCN; eluted at 0.8 mL/min with A for 25 min, then a linear gradient of B from 0-95% over 20 min, and 95% B for 15 min. ¹HNMR (CDCl₃) δ=8.21 (s, 1H, NH), 8.18 (s, 1H NH), 7.26 (m, 1H, aryl-H5), 7.20 (s, 1H, uridine-H6, ³J_{H,Sn}=18.5 Hz), 7.16 (s, 1H, uridine-H6, ³J_{H,Sn}=18.5 Hz), 6.20-6.17 (m, 1H, H1′), 5.49-5.32 (m, 2H, benzyl), 4.62-4.56 (m, 1H, H3′), 4.54-4.34 (m, 2H, H4′, H5′), 4.14-4.12 (m, 1H, H5″), 3.05 (bs, 1H, C3′-OH, exchangeable with D₂O), 2.81 (bs, 1H, C3′-OH, exchangeable with D₂O), 2.47-2.41 (m, 1H, H2′), 2.34-2.27 (m, 1H, H2″), 1.39, 1.37, 1.35 (3×s, 18H, aryl C3-3×CH₃-tBu, aryl C5-3×CH₃-tBu), 0.24 (t, 9H, 3×CH₃Sn²J_{Ssn,H}=29.0 Hz), 0.22 (t, 9H, 3×CH₃Sn ²J_{Sn,H}=29.0 Hz) ppm. ³¹P NMR (CDCl₃) δ=−8.53, −8.38 ppm. ¹¹⁹Sn NMR (CDCl₃) δ=−1.17 ppm. MSFAB-HR (m/z): [M+H]⁺calcd for C₂₇J₄₁N₂O₈PFSn, 691.1607, found 691.1640. For ¹³C peak measured 692.1653; 1.8 ppm.

Diastereomers were separated by preparative HPLC (≦440 μg of 8a per injection), using a tandem of two ACE C18, 100 Å columns (5 μm, 4.6×250 mm); eluent: solvent A 50% MeCN in water, solvent B MeCN; eluted at 0.7 mL/min with a linear gradient of B from 0-10% B over 110 min. After numerous HPLC injections, 16.4 mg a total amount of 8a fast and 23.2 mg of 8a slow was isolated. Diastereomer 8a fast eluted within 87.5-90 min and 8a slow within 90.5-93 min, past the injection. After each separation run, the isolated isomer (120-200 μg) was collected in ˜2.5 mL of eluent. The solvent was evaporated to dryness in high vacuum; the combined product residue was reconstituted in MeCN and analyzed again on the analytical HPLC (each diastereomer was ≧98% pure, UV at 220 and 280 nm). Analytical data of 8a fast are as follows: NMR (CDCl₃) δ=7.84 (s, 1H, NH), 7.26 (m, 1H, aryl-H5), 7.20 (s, 1H, uridine-H6, ³J_{H,Sn}=18.5 Hz), 6.18 (t, 1H, H1′, J=6.5 Hz), 5.46 (d, 1H, 1×H-benzyl, ²J_{H,H}=14.0 Hz), 5.34 (d, 1H, 1×H-benzyl, ²J_{H,H}=14.0 Hz), 4.62-4.58 (m, 1H, H3′), 4.44-4.41 (m, 2H, H4′, H5′), 4.13-4.11 (m, 1H, H4′), 2.51 (bs, 1H, C3′-OH, exchangeable with D₂O), 2.45-2.41 (m, 1H, H2′), 2.31 -2.25 (m, 1H, H2″), 1.37, 1.35 (2×s, 18H, aryl C3-3×CH₃-tBu, aryl C5-3×CH₃-tBu), 0.23 (t, 9H, 3×CH₃Sn, ²J_{H,Sn}=29.0 Hz) ppm. ³¹P NMR (CDCl₃) δ=−8.52 ppm. Analytical data of 8a slow are as follows: ^{I}HNMR (CDCl₃) δ=7.86 (s, 1H, NH), 7.27 (m, 1H, aryl-H5), 7.16 (s, 1H, uridine-H6, ³J_{H,Sn}=18.5 Hz), 6.16 (t, 1H, H1′, J=6.5 Hz), 5.47-5.34 (m, 2H, benzyl), 4.63-4.58 (m, 1H, H3′), 4.55-4.50 (m, 2H, H5′), 4.38-4.33 (m, 2H, H5″), 4.07-4.04 (m, 1H, H4′), 2.80 (bs, 1H, C3′-OH, exchangeable with D₂O), 2.47-2.42 (m, 1H, H2′), 2.35-2.30 (m, 1H, H2″), 1.39, 1.36 (2×s, 18H, aryl C3-3×CH₃tBu, aryl C5-3×CH₃-tBu), 0.24 (t, 9H, 3×CH₃Sn, ²J_{H,Sn}=29.0 Hz) ppm. ³¹P NMR (CDCl₃) δ=−8.39 ppm.

### Synthesis of 5-Trimethylstannyl-5′-O-[cyclo-3,5-Di-(tert-butyl)-6-fluoroSaligenyl]-3′-fluoro-2′,3% dideoxyuridine Monophosphate (24a)

General Procedure D was carried out with 5-iodo-5′-O-[cyclo-3,5-di-(tert-butyl)-6-fluorosaligenyl]-3′-fluoro-2′,3′-dideoxyuridine 24 (166 mg, 0.25 mmol), hexamethylditin (112 mg, 0.34 mmol) and the palladium(II) catalyst (36 mg, 0.052 mmol) in EtOAc (14 mL) until starting 24 despaired (˜45 min) on TLC (EtOAc/MeOH, 2:1). The crude product was initially separated and partially purified on a silica gel column (DCM/MeOH gradient, 10:0.2-0.5) and the purification was continued on the HPLC, equipped with a semi preparative Columbus C18, 100 Å (5 μm, 10×250 mm) column; eluent: solvent A 45% MeCN, solvent B MeCN; eluted with a linear gradient of B from 0-95% over 60 min, at 2.5 mL/min flow rate. Combined fractions after the repetitive evaporation from dry MeCN, gave pure stannane 24a (87 mg, 49%) as colorless foam; R_{f} value 0.84 (DCM/MeOH, 10:0.5). The HPLC analysis showed a mixture (47:53 ratio) of diastereomers: 24a fast, t_{R}=37.6 min and 24a slow, t_{R}=38.2 min (≧95% pure, UV at 280 nm); column: ACE C18, 100 Å (5 μm, 4.6×250 mm); eluent: solvent A 50% MeCN in water, solvent B MeCN; eluted with a linear gradient of B from 0-95% B over 60 min and then 95% B for 30 min at 1 mL/min. Diastereomers were not separated preparatively. ¹HNMR (DMSO-d₆) δ=11.26 (s, 1H, NH), 7.296 (s, 1H, uridine-H6, ³J_{H,Sn}=18.5 Hz), 7.293 (s, 1H, uridine-H6, ³J_{H,Sn}=18.5 Hz), 7.25 (s, 1H, aryl-H4), 7.23 (m, 1H, aryl-H4), 6.18-6.11 (m, 1H, H1′), 5.58-5.29 (m, 3H, benzyl, H3′), 4.70-4.32 (m, 3H, H4′, H5′, H5″), 2.54-2.42 (m, 2H, H2′, H2″), 1.331, 1.324, 1.319, 1.314 (overlapped s, 18H, aryl-C3-3×CH₃-tBu and aryl-C5-3×CH₃-tBu), 0.183 (t, 3×3H, 3×CH₃Sn, ²J_{H,Sn}=29.0 Hz), 0.169 (t, 3×3H, 3×CH₃Sn, ²J_{H,Sn}=29.0 Hz) ppm. ³¹P NMR (DMSO-d₆) δ=−9.48, −9.90 ppm. ¹¹⁹Sn NMR (DMSO-d₆) δ=−1.33 ppm. MSFAB-HR (m/z): [M+Li]⁺calcd for C₂₇H₃₉N₂O₇PF₂SnLi, 699.1645, found 699.1637. The ¹³C isotope peak measured 700.1682, 0.4 ppm of the expected value of 700.1679.

### Synthesis of 5-[¹²⁵I]-Iodo-5′-O-cycloSaligenyl-2′-deoxyuridine Monophosphate (6b)

General Procedure E was conducted within 0.54-11.7 mCi range, to give ˜42 mCi of 6b after six consecutive radioiodinations of the stannyl precursor 6a. An average isolated yield of the product was 88%. The latest preparation was carried out with stannane 6a (120 μg) and [¹²⁵I]NaI/NaOH (94 μL, 10.2 mCi). The HPLC purification of the product proceeded on Jupiter C18, 300 Å (5 μm, 4.6×250 mm) column; eluent: solvent A 10% MeCN in water, solvent B MeCN and 0.8 mL/min elution rate of a linear gradient of B from 0-20% over 33 min, followed by a linear gradient of B from 20-95% for 5 min, and finally 95% B for the period of 15 min. The main radioactivity peak (9.3 mCi, 91% yield) was eluted and collected in four fractions (a total volume ˜3.3 mL), within 28-32 min after the injection of 460 μL (˜10.1 mCi) of the reaction mixture. An excess of unreacted tin precursor 6a was separated from the radioiodinated product without difficulty, eluting ˜20 min later (t_{R}=50.6 min). If required, diastereomers of 6b were separated using the same HPLC conditions. The diastereomer 6b fast eluted at t_{R}=29.8 min and 6b slow at t_{R}=30.8 min. When a solution of 20% MeCN in water was used as solvent A and the column was eluted at the 0.8 mL/min flow rate with solvent A for the period of 25 min, followed by a linear gradient of B from 0-95% over 10 min, and finally with 95% B for 10 min; diastereomers were eluted faster and fully separated: 6b fast, t_{R}=20.6 min and 6b slow, t_{R}=22.4 min. In a single HPLC run, the complete separation (each diastereomer≧98% pure, Bioscan NaI(T) detector) was achieved, if a total amount of injected 6b was ≦230 μCi. Larger batches of individual diastereomers were acquired by repeating the HPLC injections, or using a semi preparative column: Columbus C18, 100 Å (5 μm, 10×250 mm), eluted at the 2.6 mL/min flow rate with a 20% MeCN solution in water. Diastereomer 6b fast eluted within 75-77 min and 6b slow 83-87 min, past the injection, and each was collected in ˜8 mL volume of an eluent. The solvent was evaporated to dryness in high vacuum at 30° C., using the SpeedVac system. The HPLC co-injections of the purified 6b with its nonradioactive analog 6, and the parallel monitoring of the radioactivity and UV signal, confirmed the identical elution of both compounds.

### Synthesis of 5-[¹²⁵I]-Iodo-5′-O-cyclo(3-methylSaligenyl)-2′-deoxyuridine Monophosphate (7b)

General Procedure E was conducted within 0.52-10.1 mCi range, to give ˜33 mCi of 7b in six consecutive radioiodinations of the stannyl precursor 7a. An average isolated yield was 81%. The latest radioiodination was performed with stannane 7a (˜110 μg) and [¹²⁵I]NaI/NaOH (90 μL, 9.3 mCi). The HPLC purification of the product continued on Jupiter C18, 300 Å (5 μm, 4.6×250 mm) column; eluent: solvent A 18% MeCN in water, solvent B MeCN; and 0.8 mL/min flow rate. The column was eluted with solvent A for the period of 30 min, then with a linear gradient of B from 0-95% over 10 min, and 95% B for 20 min. The product 7b (8.1 mCi, 87%), which eluted within 21.5-24.7 min after the injection of 410 μL (˜9.1 mCi) of the reaction mixture, was collected in three fractions (2.5 mL a total volume). An excess of unreacted tin precursor 7a was fully separated, eluting between 27.3-27.7 min. The HPLC co-injections of the purified 7b with its nonradioactive analog 7, and monitoring the radioactivity (Bioscan NaI(T) detector) and UV signal at 280 nm, verified the same HPLC mobility of both compounds. Diastereomers of 7b were separated on Jupiter C18, 300 Å (5 μm, 4.6×250 mm) column, eluted with 20% MeCN solution in water, for the period of 45 min at the 0.8 mL/min flow rate. Individual diastereomers: 7b fast (t_{R}=25.3 min) and 7b slow (t_{R}=26.8 min) were ≧98% pure (Bioscan NaI(T)). In the course of a single HPLC run, the full separation of diastereomers was limited to the total of ˜270 μCi 7b loaded onto a column. Larger batches of resolved diastereomers were attainable through the repetitive HPLC injections, or using a larger column: Columbus C18, 100 Å (5 μm, 10×250 mm); eluent: a solution of 22% MeCN in water and the flow rate of 2.5 mL/min. Diastereomer 7b fast eluted within 72-77 min, and 7b slow within 79-81 min past the injection. The solvent was evaporated to dryness in high vacuum, on the SpeedVac system. The product residue was analyzed again on the analytical HPLC shortly before conducting scheduled experiments and was reconstituted in an appropriate solvent.

### Synthesis of 5-[¹²⁵I]-Iodo-5′-O-[cyclo-3,5-di-(tert-butyl)-6-fluoroSaligenyl]-2′-deoxyuridine Monophosphate (8b)

The overall 52 mCi of 8b was acquired in eleven successive radioiodinations, using one of the purified tin precursors: 8a, 8a fast or 8a slow, and conducting General Procedure E within the 0.25-10.7 mCi range. An average isolated yield of the product was 88%. The latest radiolabeling was performed with diastereomeric stannane 8a (˜115 μg) and [¹²⁵I]NaI/NaOH (40 μL, 3.91 mCi). The HPLC purification of the product proceeded on ACE C18, 100 Å (5 μm, 4.6×250 mm) column; eluent: solvent A 50% MeCN in water, solvent B MeCN; and the flow rate of 1.0 mL/min. A column was eluted with solvent A for the period of 60 min, then with a linear gradient of B from 0-95% over 10 min, and 95% B for 20 min. The product 8b (3.48 mCi, 89%), which eluted within 25-29 min after the injection of 350 μL (˜3.76 mCi) of the reaction mixture, was collected in four fractions. An excess of unreacted stannane 8a was eluting ˜15 min later. The mixture of purified 8b (˜12 μCi, 10 μLt) and the corresponding nonradioactive analog 8 (˜15 μg, 20 μL) was prepared in acetonitrile, and injected onto the HPLC, using the same column and conditions as during the separation of the product. Both compounds eluted together, showing the identical retention times. Diastereomers of 8b were separated on ACE C18, 100 Å (5 μm, 4.6×250 mm) column, eluted at the 0.8 mL/min flow rate, with a 45% MeCN solution in water, for the period of 45 min. Each diastereomer: 8b fast (t_{R}=30.3 min) and 8b slow (t_{R}=32.8 min), was ≧98% pure (Bioscan NaI(T)). The complete separation of diastereomers in the single HPLC run was reached, if the total amount of 8b loaded onto the column was ≦220 μCi. Larger batches of the individual diastereomers were obtained by the repetitive HPLC injections of purified 8b, or by conducting the radioiodination using a single diastereomer of the tin precursor 8a fast or 8a slow. Fractions containing the product were combined and the solvent was evaporated in high vacuum on the SpeedVac system. The product residue was reconstituted in MeCN and analyzed again on the analytical HPLC, shortly before conducting planned experiments.

### Synthesis of 5-[¹²⁵I]-Iodo-5′-O-[cyclo-3,5-Di-(tert-butyl)-6-fluoroSaligenyl]-3′-fluoro-2′,3′-dideoxyuridine (cycloSal[¹²⁵I]IUdRFMP) (24b).

The overall amount of prepared 24b was 10.4 mCi, acquired in four consecutive radioiodinations. General Procedure E was carried out within the 0.5-5.2 mCi range and an average isolated yield was 93%. The largest conducted radiolabeling proceeded with stannane 24a (˜120 μg) and [¹²⁵I]NaI/NaOH (60 μL, 5.2 mCi). The HPLC purification of the crude product was best achieved on ACE C18, 100 Å (5 μm, 4.6×250 mm) column; eluent: solvent A 50% MeCN in water, solvent B MeCN. A column was eluted at 1.0 mL/min of the flow rate, with a linear gradient of B from 0-95% over 60 min, followed by 95% B for the period of 30 min. The product 24b (4.85 mCi, 92%) collected within 26-28 min after the injection of 500 μL (˜5.1 mCi) of the reaction mixture, was fully separated from an excess of the tin precursor 24a, which eluted ˜9 min later (37.0-37.6 min). Fractions containing the product were combined, solvent evaporated with a stream of nitrogen, and the residue further dried in high vacuum. The mixture of purified 24b (˜12 μCi, 10 μL) and its nonradioactive analog 24 (˜15 μg, 20 μL) was prepared in acetonitrile and analyzed on the HPLC, using the same setting as during the separation of the product. The analysis showed diastereomer 24b fast at t_{R}=26.1 min and 24b slow; t_{R}=26.7 min, co-eluting with the [¹²⁷I]-iodoanalog: 24 fast, t_{R}=25.9 min and 24 slow, t_{R}=26.5 min. Diastereomers of 24b were separated on ACE C18, 100 Å (5 μm, 4.6×250 mm) column; eluent: solvent A 50% MeCN in water, solvent B MeCN. A column was eluted with a linear gradient of B from 0-10% over 60 min at the 0.8 mL/min flow rate. Each diastereomer, 24b fast (t_{R}=38.1 min) and 24b slow (t_{R}=40.7 min), was ≧98% pure (Bioscan NaI(T) detector). In the single HPLC run a full separation was possible, if the total amount of 24b loaded onto a column was ≦120 μCi. Larger lots of single diastereomers were obtained by the repetitive HPLC injections or using a larger column: Columbus C18, 100 Å (5 μm, 10×250 mm); eluted at the 2.2 mL/min flow rate with 57% MeCN in water.

### EXAMPLE 2

### Chromatographic resolution of fast and slow cycloSaligenyl-phophotriester diastereomers

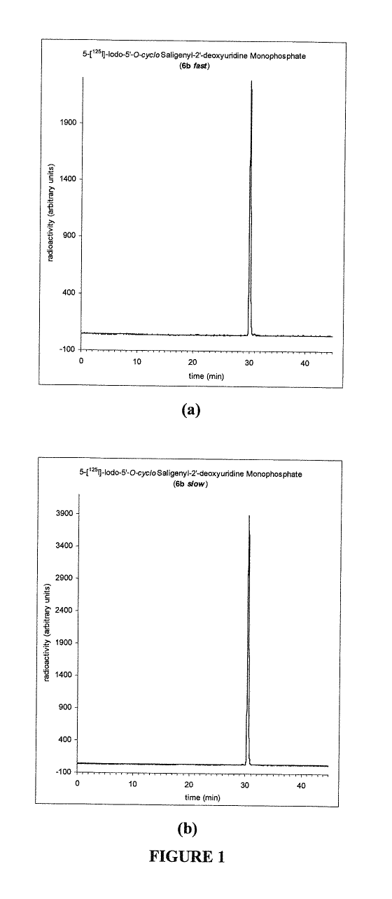

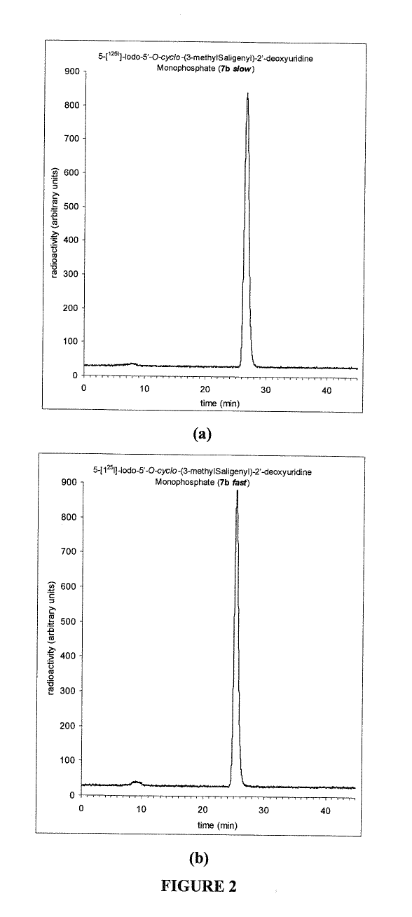

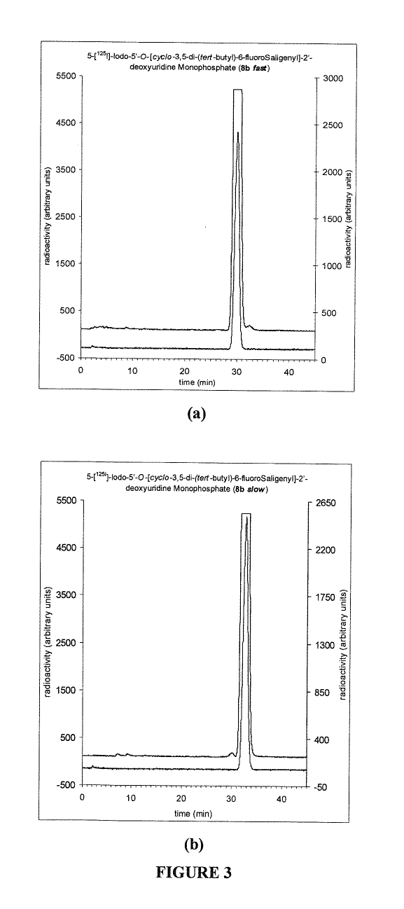

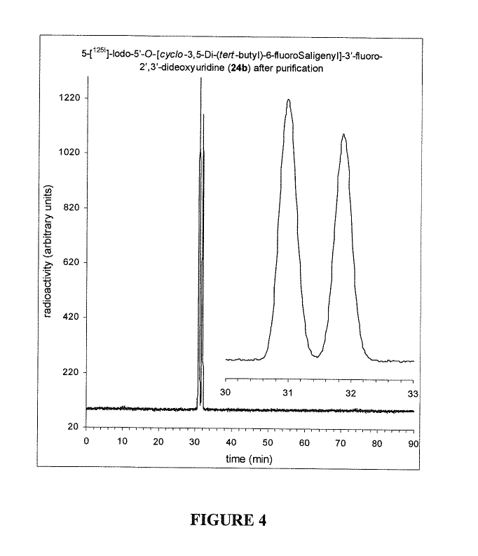

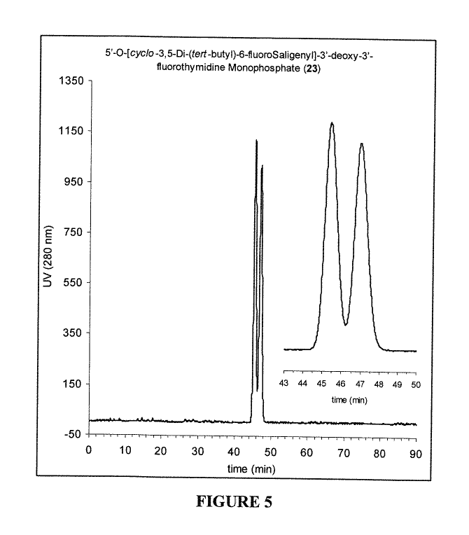

Since the synthesis of cycloSaligenyl-phosphotriesters is not stereoselective, all products are obtained as mixtures of two diastereomers (Sₚ and Rₚ configuration, approximately 1:1 ratio) at each phosphorus center. The diastereomers are differentiated and named as the -fast and the -slow diastereomer, in correlation to the HPLC retention time (t_{R}) of each isomer. Only a tentative assignment of the Rₚ/Sₚ stereochemistry at the phosphorus can be made, by the association to previously synthesized cycloSal-compounds, with already known stereochemical configuration (Balzarini J, Aquaro S, Knispel T, Rampazzo C, Bianchi V, Perno C F, De Clercq E, Meier C. Cyclosaligenyl-2′,3′-didehydro-2′,3′-dideoxythymidine monophosphate: efficient intracellular delivery of d4TMP. Mol Pharmacol. 2000 November; 58(5):928-35), as a reference. Diastereomers of all synthesized 5′-O-cycloSaligenyl-phosphotriesters could be separated by the reverse phase HPLC. The compounds of the present invention, 6b-14b, 23, and 24b were analyzed by reverse phase HPLC to determine their diastereomeric purity (FIGS. 1-5).

The individual [¹²⁵I]-radioiodinated diastereomers of 5′-O-cycloSal-triesters 6b-8b and 3′-O-cycloSal-triesters 12b-14b were prepared in one of two accessible ways: 1) conducting [¹²⁵I]-iododestannylation with a single diastereomer of trimethylstannyl-precursors 6a slow-8a slow or 6a fast-8a fast, or by 2) using a diastereomeric mixture of stannane and the separation of isomers during a final purification of the [¹²⁵I]-radioiodolabeled product. The complete separation of [¹²⁵I]-labeled diastereomers was limited however, to a total of ˜560 μCi injected in a single HPLC run. Therefore, if a larger batch of a single [¹²⁵I]-iodolabeled diastereomer was required; the individual diastereomer of trimethyltin precursor was preferred, to speed up the preparation. Diastereomers of ¹²⁵I-radioiodolabeled cycloSaligenyl-triesters were regularly obtained in quantities of up to 10 mCi, starting with the selected diastereomer of appropriate tin precursor.

All 3′,5′-O,O-di-cycloSaligenyl-phosphotriesters 9-11 products, with two different stereogenic centers formed during the synthesis, were expected to exist as mixtures of four stereoisomers (with configurations: Sₚ/Sₚ, Rₚ/Sₚ, Sₚ/Rₚ and Rₚ/Rₚ) all in a ratio of 1:1. Indeed, the HPLC analysis and ³¹P NMR spectroscopy of 3′,5′-di-substituted iodides 9-11 and stannanes 9a -11a pointed out to mixtures of four diastereomers. These diastereomers were practically inseparable and all of the applied HPLC methods led only to a partial separation of the slowest or the fastest isomer. Consequently, the ¹²⁵I-labeled target 3′,5′-O,O-dicycloSaligenyl-phosphotriesters 9b-11b were purified and isolated as mixtures of diastereomers.

### EXAMPLE 3

### Synthesis of a O-succinyl-dihydrotestosterone analog of a corresponding 5′-O-cyclosaligenyl-2′-deoxyuridine monophosphate

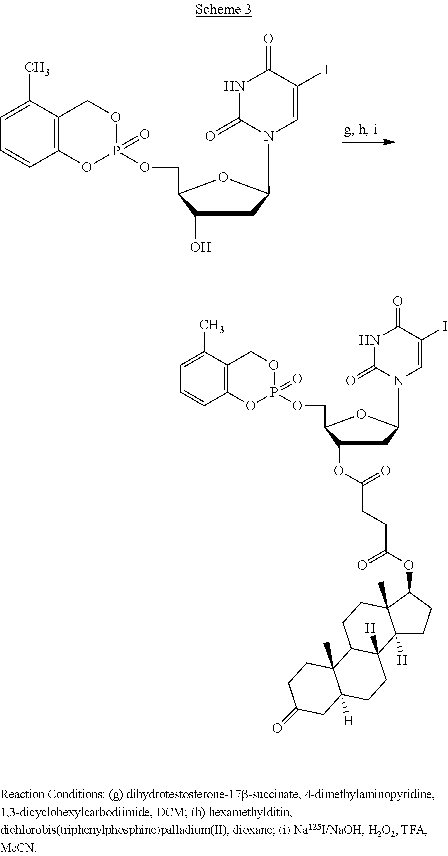

Nonradioactive analogues, containing the androstan-3-one moiety were all prepared by esterification of dihydrotestosterone 17β-succinate with the corresponding 5′-O-cycksaligenyl-2′-deoxyuridine monophosphates (Scheme 3). Synthesis of radiolabelled analogues was based on the non-carrier-added electrophilic destannylation of the related trialkyl-organotin precursors, which were prepared by the stannylation of iodouridines, using hexamethylditin, and were carried out in the presence of palladium(II) catalyst.

### Synthesis of 5-Iodo-5′-O-cyclo(3-methylsaligenyl)-3′-O-(17β-succinyl-5α-androstan-3-one)-2′-deoxyuridine Monophosphate

To a stirred solution of dihydrotestosterone 17β-succinate (0.50 g, 1.30 mmol) and 5-iodo-5′-O-cyclo(3-methylsaligenyl)-2′-deoxyuridine monophosphate (0.70 g, 1.31 mmol) containing 4-dimethylaminopyridine (0.017 g, 0.14 mmol) in dry dichloromethane (35 mL) at 0° C., 1,3-dicyclohexylcarbodiimide (0.29 g, 1.40 mmol) was added. The solution was warmed slowly (1 h) to room temperature and stirring continued for an additional 2 h (TLC monitoring). The mixture was diluted with n-hexane/CH₂Cl₂ (3:2, v/v) mixture (40 mL) and filtered. The filtrate was washed consecutively with 5% aqueous citric acid (20 mL), 10% NaHCO₃ (20 mL), and water (2×25 mL) and dried over MgSO₄. The solvent was removed under reduced pressure. The resulting crude product was purified by column chromatography on a silica gel (CHCl₃/CH₃OH gradient, 10:0.2-0.4) to give a title compound (0.88 g, 74%) as a colorless foam. The HPLC analysis did not indicate a presence of diastereomers: t_{R}=33.4 min (≧97% pure, UV at 220 and 280 nm). The analysis was conducted using Jupiter C18, 300 Å (5 μm, 4.6×250 mm) column; eluent: solvent A 20% MeCN in water, solvent B MeCN; and a column eluted at 0.8 mL/min with a linear gradient of B from 0-95% over 40 min, and 95% B for the period of 20 min. ¹H NMR (DMSO-d₆) δ=11.73, 11.68 (2 s, 1H, NH), 7.99, 7.86 (2 s, 1H, uridine-H₆), 7.25-7.21 (m, 1H, aryl-H5), 7.12-7.07 (m, 2H, aryl-H6, aryl-H4), 6.08, 6.05 (dd, 1H, H1′), 5.51-5.39 (m, 2H, benzyl), 4.62 (t, 1H, H17-DHT, J=8.5 Hz), 4.34-4.26 (m, 1H, H5″, H3′), 4.22-4.18 (m, 1H, H5′), 3.93-3.89 (m, 1H, H4′), 2.71-2.61 (m, 4H, H2 and H3 succinyl), 2.45-2.39 (m, 4H, H2″), 2.32-0.76 (m, 28H, from DHT with 1.13 (s), 3H, H18-DHT, 0.82 (s), 3H, H19-DHT), 2.23 (s, 3H, C3-aryl-CH₃), 2.10-2.05 (m, 1H, H2″) ppm. ³¹P NMR (DMSO-d₆) δ=−8.89, −8.93 (2 s, diastereomeric mixture) ppm. MSFAB-HR (m/z): [M+H]⁺calcd for C₄₀H₅₁N₂O₁₂PI, 909.2146 found 909.2099.

### Synthesis of 5-Trimethylstannyl-5′-O-cyclo(3-methylsaligenyl)-3′-O-(17β-succinyl-5α-androstan-3-one)-2′-deoxyuridine Monophosphate

A solution of 5-iodo-5′-O-cyclo(3-methylsaligenyl)-3′-O-(17β-succinyl-5α-androstan-3-one)-2′-deoxyuridine monophosphate (0.34 g, 0.374 mmol), hexamethylditin (0.21 g, 0.60 mmol) and dichlorobis(triphenylphosphine)palladium(II) (0.024 g, 0.03 mmol) in dioxane (14 mL) was refluxed under a nitrogen atmosphere, until the starting iodide disappeared (about 2h). Dioxane (20 mL) was added after cooling and the reaction mixture was filtered through a MgSO4 (4 g) plug and the filtrate was evaporated under reduced pressure and the resulted residue kept under high vacuum, was purified further by column chromatography on a silica gel (CHCl₃/CH₃OH gradient, 10:0.1-0.4) and gave a crude product (0.31 g, 87% yield) as a colorless foam. The collected was ˜89% pure by HPLC analysis. Final purification was accomplished by preparative HPLC of a crude product (286 mg, 66 mg per injection), on Columbus C18, 100 Å (5 μm, 10×250 mm) column eluted at 2.5 mL/min with 67% MeCN solution in water. Combined fractions were evaporated to dryness in high vacuum; the product residue (143 mg, 40%) was reconstituted in MeCN and analyzed again on the analytical HPLC. The HPLC analysis did not indicate diastereomers: t_{R}=36.8 min (≧97% pure, UV at 280 nm). The analysis was conducted using Jupiter C18, 300 Å (5 μm, 4.6×250 mm) column; eluent: solvent A 20% MeCN in water, solvent B MeCN; and a column eluted at 0.8 mL/min with a linear gradient of B from 0-95% over 40 min, and 95% B for the period of 20 min. Analytical data are as follows: ¹HNMR (CDCl₃) δ=8.13 (s, 1H, NH), 8.11 (s, 1H NH), 7.33 (s, 1H, uridine-H6, ³J_{H,Sn}=18.4 Hz), 7.29 (s, 1H, uridine-H6, ³J_{H,Sn}=18.4 Hz), 7.17 (m, 1H, aryl-H5), 7.12-7.04 (m, 3H, aryl-H3, aryl-H4, aryl-H6), 6.20-6.15 (m, 1H, H1′), 5.41-5.34 (m, 2H, benzyl), 4.62 (t, 1H, H17-DHT, J=8.5 Hz), 4.55-4.52 (m, 1H, H3′), 4.47-4.39 (m, 2H, H4′, H5′), 4.16-4.11 (m, 1H, H5″), 2.69-2.58 (m, 4H, H2 and H3 succinyl), 2.45-2.36 (m, H1, H2′), 2.34-2.27(m, 1H, H2″), 2.32-0.74 (m, 28H from DHT), 2.21-2.19 (m, 3H, aryl-C3CH₃), 0.49 (s, 9H, 3×CH₃, ²J_{Sn,H}=29.2 Hz) ppm.³¹P NMR (CDCl₃) δ=−8.39, −8.21 ppm. ¹¹⁹Sn NMR (CDCl₃) δ=−1.09 ppm. MSFAB-HR (m/z): [M+H]⁺calcd for C₄₃H₅₉N₂O₁₂PSn, 947.2828, found 947.3012.

### Synthesis of 5-[¹²⁵I]Iodo-5′-O-cyclo(3-methylsaligenyl)-3′-O-(17β-succinyl-5α-androstan-3-one)-2′-deoxyuridine Monophosphate

Into a glass tube containing 5-trimethylstannyl-5′-O-cyclo(3-methylsaligenyl)-3′-O-(17β-succinyl-5α-androstan-3-one)-2′-deoxyuridine monophosphate (120 μg, ˜150 μmol) dissolved in MeCN (50 μL), a solution of Na¹²⁵I/NaOH (10 μL, 0.6-2.0 mCi) was added, followed by a 30% water solution of H₂O₂ (5 μL), and by TFA solution (50 μL, 0.1 N in MeCN) added with a 2 min delay. The mixture was briefly vortexed and left for 15 min at room temperature. The reaction was quenched with Na₂S₂O₃ (100 μg in 100 μL of H₂O) and taken up into a syringe. The reaction tube was washed twice with 50 μL of H₂O/MeCN (9:1) solution. The previously withdrawn reaction mixture, plus washes were injected onto the HPLC system and separated, by means of Jupiter C18, 300 Å (5 μm, 4.6×250 mm) column; eluent: solvent A 50% MeCN in water, solvent B MeCN; and a column eluted at 0.8 mL/min with a linear gradient of B from 0-50% over 45 min. The eluent from a column (1 mL fractions collected) was monitored using a radioactivity detector, connected to the outlet of UV detector (detection at 220 and 280 nm). The reaction was conducted four times within 0.64-1.87 mCi range and an average isolated yield of the product was 88%. The main radioactivity peak (80-91%) was eluted and collected in four fractions (a total volume ˜3.3 mL), within 29.5-33 min after the injection of 460-500 μL of the reaction mixture. An excess of unreacted tin precursor was separated from the radioiodinated product without difficulty, eluting ˜12 min later. Eluted fractions containing a product, combined and evaporated with a stream of dried nitrogen, were reconstituted in MeCN (˜1 μCi/1 μL concentration) and were filtered through a sterile (Millipore 0.22 gm) filter into a sterile evacuated vial. Identity of radiolabeled product was confirmed by evaluating the UV signal of nonradioactive iodo-analog with the radioactivity signal of the product, from the radio-HPLC analysis. The specific activities were determined by the UV absorbance of radioactive peaks, as compared to the standard curves of unlabeled reference compound.

### EXAMPLE 4

### Determination of BChE Inhibition (IC₅₀) by Representative Compounds of Formula (I)

Assays employed to determine IC₅₀ were developed by the present inventors and utilized UV based detection. For UV-based assays, a BChE solution in 0.1 M potassium phosphate, pH 7.0 was placed in the desired numbers of wells of a 96-well plate (0.05 mL/well). The investigated compound was diluted in DMSO to produce concentrations from 0 to 10 μM in DMSO and 0.002 mL/well of these dilutions was added to BChE-containing wells. Reaction mixtures were incubated at room temperature for 30 min. The reagent consisting of BChE substrate, 1 mM (2-mercaptoethyl)trimethylammonium iodide butyrate, and 0.5 mM 5,5′-dithio-bis(2-nitrobenzoic acid) in 0.1 M potassium phosphate, pH 7.0 (prepared fresh for each assay) was added, 0.25 mL per well. The mixture was incubated at room temperature and the OD at λ=405 nm was read at 1, 2, 3, 4, 5, 10, and 15 min after the addition of this reagent using the Opsys MR™plate reader (Dynex Technologies, Chantilly, Va.). Data were analyzed using the IC₅₀ nonlinear regression function provided by GraphPad Prism (GraphPad Software, La Jolla, Calif.).

The compound binding of several non-radioactive compounds of the method of the invention were analyzed by the aforementioned human BChE compound binding study to determine their affinity for the therapeutic target (Table 1).

TABLE 1

| Compound | IC₅₀ (nM) |
|---|---|
| 6 - fast | 1284 |
| 6 - slow | 9.2 |
| 7 - fast | >7,000 |
| 7 - slow | 61.2 |
| 8 - fast | >60,000 |
| 8 - slow | 50.1 |
| 24 - fast | 549.2 |
| 24 - slow | 19.8 |
| 23 - fast | 202.6 |
| 23 - slow | 42.6 |

The binding study demonstrates a definite preferential trend for the slow isomer rather than the fast isomer of the given compounds of the invention.

EXAMPLE 5

### Biodistribution and Pharmacokinetics of Representative Radioactive Compounds of Formula (I)

The in vivo properties and pharmacology of several compounds of the present invention were initially tested in athymic mice bearing subcutaneous LS174T tumors (human colorectal adenocarcinoma), which have several desirable properties such as rapid growth when implanted subcutaneously (SQ), good vascularization and the availability of considerable historical data for the parent compound IUdR. The clearance rates from blood, tumor, and associated organs were analyzed to determine the structure-pharmacokinetics relationship. The first compounds studied were 6b-fast and 6b-slow. Four-to-six-weeks old female athymic NCr-nu/nu mice (NCI-Frederic, MD, USA) were allowed to acclimate in Applicants' facility after delivery for no less than 5 days. All protocols involving animals were approved by the University of Nebraska Institutional Animal Care and Use Committee. Mice were housed in microisolator cages with free access to sterilized standard rodent diet and water. LS174T cells in 0.1 mL of serum-free medium were implanted SQ at 5×10⁶ cells/mouse. One week later identification transponders were implanted, also SQ. Body weights and tumor sizes were monitored twice weekly, and approximately 10 days after the cell implant, mice were randomized for biodistribution studies.

Each of the two isomers, 6b-fast and 6b-slow, was tested independently in separate groups of mice. The comparison to the parent compound, IUdR, was made by co-injecting ¹³¹IUdR with a particular ¹²⁵I-labeled diastereomer. Compounds were administered intravenously (IV) via a tail vein.

Biodistribution was conducted at 1 h, 4 h, 24 and 48 h after the administration of radiolabeled compounds (n=6 per time point). The injection doses contained approximately 1 μCi (37 kBq) ¹³¹IUdR and 5 μCi (185 kBq) ¹²⁵I-labeled 6b-fast or 6b-slow dissolved in 0.2 mL phosphate buffered saline containing 0.1% bovine serum albumin, pH 7.2 (PBS). All syringes were weighted after loading with the compound solution and immediately after the injection to determine the weight of the injected dose. Triplicate standards of the injected dose were prepared in PBS and counted in a gamma counter just before the beginning of the experiment. The radioactive content of these standards was also determined alongside all tissues after the necropsy to correct the tissue uptake for the decay of the radioisotope. Blood, liver, spleen, heart, lungs, kidneys, brain, tumor and tail were collected during necropsy. Tissues were rinsed in ice-cold saline, patted dry and weighed. The tail was collected to validate the quality of the IV injections.

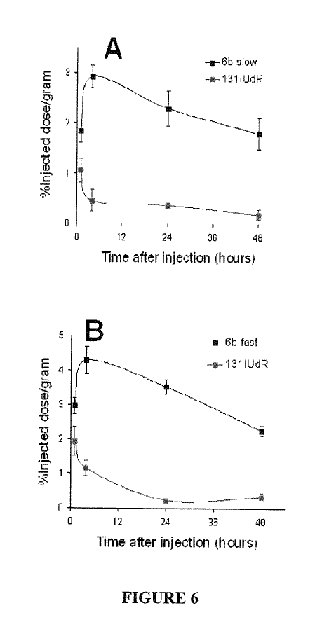

Clearance curves of 6b slow (FIG. 6A) and 6b fast (FIG. 6B) in tumor, harvested from athymic mice bearing SQ LS174T xenografts were prepared. The most significant finding shown in FIG. 6 is that the tumor uptake and retention of 6b is nearly 14× and 7× higher compared to the tumor uptake of the parent drug ¹³¹IUdR at 24 hours and 48 hours after administration, respectively. Liver and lungs also retain high levels of radioactivity. The clearance from these tissues appears to parallel the levels of 6b in blood.

Biodistribution studies of 6b slow, 7b fast, 7b slow, 8b fast, and 8b slow have also been completed. The in vivo fate 7b fast and 7b slow is very similar to the behavior of 6b fast and 6b slow and the observed differences follow the hydrophobicity of these two derivatives.

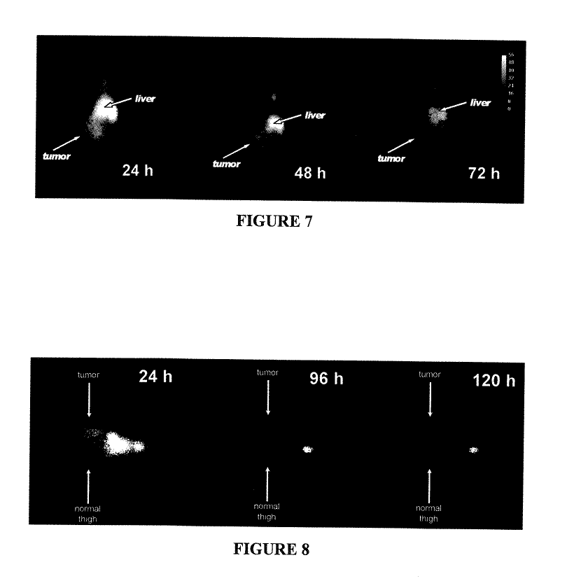

FIG. 7 presents whole body images for a typical time course distribution (24 h, 48 h, and 72 h) of 6b administered as a diastereomeric mixture in LS174T-bearing athymic mice. The images were acquired 1, 2, and 3 days after the administration of the compound. It is apparent that the radioactivity in tumor and several normal organs persists, however, it is also apparent that normal organs such as liver clear the compound at a much faster rate than the tumor, which retains the compound. It is also apparent that based on pharmacokinetics, the use of these compounds can be tailored to a specific malignancy and its anatomical location.

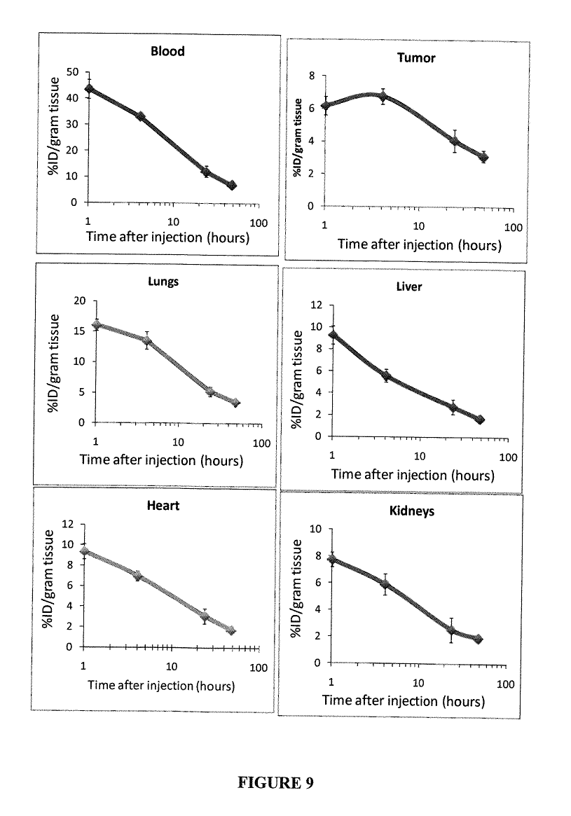

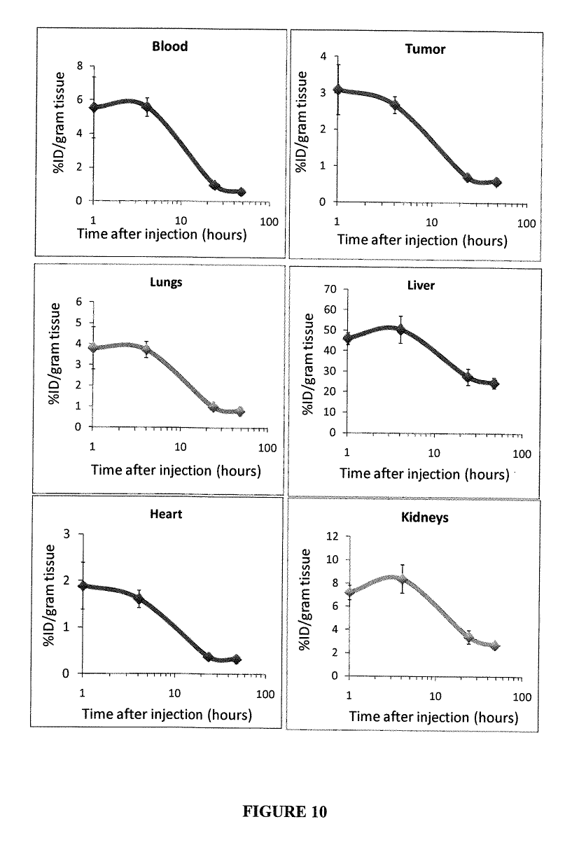

Images shown in FIG. 8 illustrate the fate of 8a fast in LS174T tumor model indicating that the compounds with lower BChE activity are useful in rapidly proliferating tumors. Compounds 8b fast and 8b slow have a distinct distribution pattern, unlike any of the other isomers. The distinct behavior of these compounds in the tumor, blood, and several normal tissues is illustrated in FIGS. 9 and 10 for compounds 8b fast and 8b slow, respectively. Based on the biodistribution and imaging studies, it is apparent that depending on the application, the compounds of the invention have “ideal” in vivo properties, i.e., 8b slow has a rapid clearance from blood and normal tissues and therefore it is suitable for the imaging of tumor response to therapy or for cancer diagnosis via a systemic administration. Other compounds have prolonged presence and therefore are more suited for loco-regional administration and are ideal for therapy.

Four compounds, 6b fast, 6b slow, 7b fast, and 7b slow, were initially selected for a pilot therapy protocol in the intraperitoneal model of ovarian adenocarcinoma using OVCAR-3-bearing athymic mice. Based on similar biodistribution of 6b fast, 6b slow, and 7b fast, 7b slow, pairs, the therapy was conducted using the diastereomeric mixtures. 8b fast and 8b slow are evaluated individually in therapy trials. It was particularly important to have these two isomers separated because their in vivo behavior is radically different, including their blood clearance, and liver and lung uptake levels after the IV administration.

### EXAMPLE 6

### Therapy Studies Utilizing Compounds of Formula (I) in an Ovarian Cancer Tumor Model

In pilot therapy experiments, the effects of unmodified ¹²⁵IUdR on the growth of IP OVCAR-3 tumors were compared to the therapeutic effects of the 6b diastereomeric mixture and 7b diastereomeric mixture. The tumor model used in this study, OVCAR-3 cell line, was established from the malignant ascites of a patient with progressive adenocarcinoma of the ovary after many types of chemotherapy including cyclophosphamide, adriamycin, and cisplatin. OVCAR-3 is resistant in vitro to clinically relevant concentrations of adriamycin, melphalan, and cisplatin (Hamilton T C, Young R C, Louie K G, Behrens B C, McKoy W M, Grotzinger K R, Ozols R F. Characterization of a xenograft model of human ovarian carcinoma which produces ascites and intraabdominal carcinomatosis in mice. Cancer Res. 1984; 44:5286-90.). In immunodeficient mice, IP injected OVCAR-3 cells produce malignant ascites, peritoneal carcinomatosis, and serosal and visceral seeding that, if left untreated, leads to death from respiratory compromise, hemorrhage from invasion of intra-abdominal blood vessels, and bowel obstruction. Substantial literature data (nearly 100 publications) confirm that this tumor model has pathogenesis and metastatic properties similar to those of human ovarian cancer.

Mice received IP implants of approximately 2.4×10⁸ OVCAR-3 cells/mouse isolated from fresh OVCAR-3 mouse ascites. Cell viability was measured before and after injection, and was >90%. Four days after tumor cells were implanted, the mice received SQ transponders and were randomized via a lottery into four groups: NT—untreated controls that received IP injection of PBS; 7b group—receiving IP doses of the 7b diastereomeric mixture in PBS (average 0.4 mCi/mouse (14.8 MBq)); 6b group—receiving IP doses of the 6b diastereomeric mixture in PBS (average dose 0.36 mCi/mouse (13.3 MBq)); and ¹²⁵IUdR group—receiving IP doses of¹²⁵IUdR (average 0.43 mCi/mouse (15.9 MBq)) in PBS. Mice were monitored three times per week.

When the body weight and the gross observation of the mice, including the palpitation of the abdomen, indicated that some solid tumors are beginning to form, mice were given a boost dose of the compounds as follows: 26 days after the first dose mice in the 7b group were treated with additional 0.18 mCi/mouse (6.6 MBq) 7b diastereomeric mixture in PBS. Mice in the 6b group received 0.22 mCi (8.1 MBq) 6b diastereomeric mixture on day 27; and on day 28 mice in ¹²⁵IUdR group were treated with additional 0.2 mCi (7.4 MBq)¹²⁵1UdR. Slight differences in the dosing schedule and the administered doses are because of the required large quantities of the compounds. For these experiments, ˜20 mCi of no-carrier added and radiochemically pure compounds were required. Each agent was purified on the analytical reversed phase HPLC column. The use of preparative columns in this case is impractical because the products are recovered in large volumes of acetonitrile-containing solvent that must be evaporated to dryness before the use in mice. The resolution on the analytical column is superior and the collected volumes much smaller.

Overall, mice in the 7b group were treated with an average of 0.58 mCi (21.5 MBq) 7b diastereomeric mixture; mice in the 6b group were treated with an average of 0.58 mCi (21.5 MBq) 6b diastereomeric mixture, and mice in the ¹²⁵IUdR group were treated with an average of 0.63 mCi (23.3 MBq) ¹²⁵IUdR.

The therapy was terminated 7 weeks after the first dosing. Mice were sacrificed for the biodistribution study. Blood and solid tumor were taken for the evaluation of their radioactive content. The peritoneal cavity was lavaged with 2-mL aliquots of PBS, and PBS wash and ascites were collected. One mL of the ascites suspension with cancer cells was taken for gamma counting. The rest of the abdominal fluid was centrifuged and 0.5 mL of the supernatant was reserved for gamma counting. The weight of the cell pellet and the supernatant were determined.

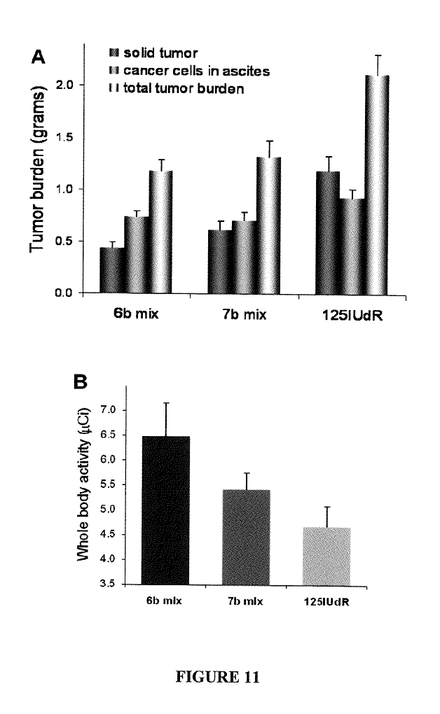

FIG. 11A summarizes the weights of solid tumors as the tumor burden in addition to the cell pellets recovered from the abdominal cavities of these mice. There is a significant reduction, ˜50%, in solid tumor deposits in the 6b-treated and 7b-treated mice as compared to ¹⁵IUdR. The statistical analyses of these data are shown in Table 2.

TABLE 2

| | | Weights | | % | % |
|---|---|---|---|---|---|
| | Weights | of Cell | % | ID/g in | ID/g in |
| | of Solid | Pellet from | ID/g in | Solid | Cells in |
| t-Test | Tumors | Ascites | Blood | Tumors | Ascites |
| 6b v. 7b | 0.93 | 0.39 | 0.016 | 0.14 | 0.78 |
| 6b v. ¹²⁵IUdR | 0.005 | 0.17 | 9.4E−10 | 4.4E−08 | 0.0047 |
| 7b v. ¹²⁵IUdR | 0.002 | 0.05 | 0.001 | 0.007 | 0.03 |

Two weeks after the first dose of radiolabeled compounds, the whole body radioactivity was measured to determine to what extent the additional doses of the compounds may increase the radiation burden and therefore may be detrimental to the animal health (FIG. 11B). The absolute amounts were low and ranged from ˜6.5 μCi for the 6b group to ˜4 μCi in the 7b group. It also appears that notwithstanding great similarities in the in vivo distribution of 6b fast, 6b slow and 7b fast, 7b slow, the whole body retention after 14 days is distinctly higher for the 6b group, with the following specific values: 1.87 (0.16) % ID (median 1.89% ID) for 6b; 1.21 (0.12) % ID (median 1.25% ID) for 7b; and 0.97 (0.11) % ID (median 1.08% ID) for ¹²⁵IUdR. The corresponding P values are 0.003, 0.0001, 0.166 for 7b vs. 6b, 6b vs. ¹²⁵IUdR, and 7b vs. ¹²⁵IUdR, respectively.

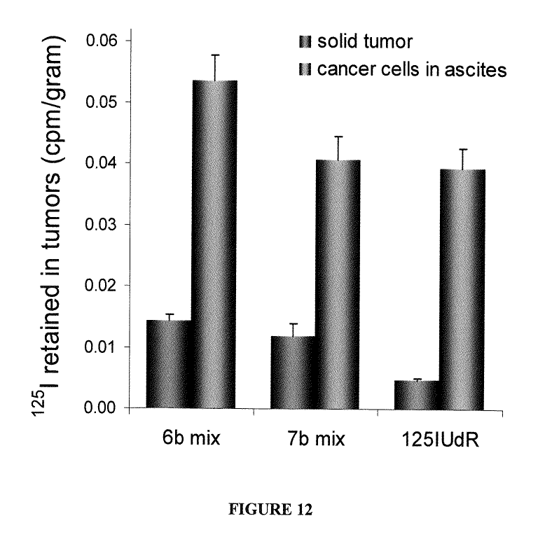

The data collected after necropsy conducted 4 weeks after the boost dose indicated that the tumor retention of the 6b and 7b was significantly higher compared to ¹²⁵IUdR (FIG. 12). Percent injected doses (ID) shown in FIG. 12 were calculated based on the cumulative administered dose and are uncorrected for decay.

### EXAMPLE 7

### Therapy with Fractionated Doses of Compound 8b-slow in Advanced OVCAR-3 Tumors

Biodistribution data for 8b showed a very favorable blood clearance curve (FIGS. 9 and 10). It also indicated that the liver is the major site of the normal tissue uptake. Therefore, it was expected that 8b slow might prove to be an excellent choice for the IP therapy. Having already established that four compounds are effective in the early stages of the OVCAR-3 tumor development on athymic mice, this therapy was designed to commence at the advanced stages of the disease.

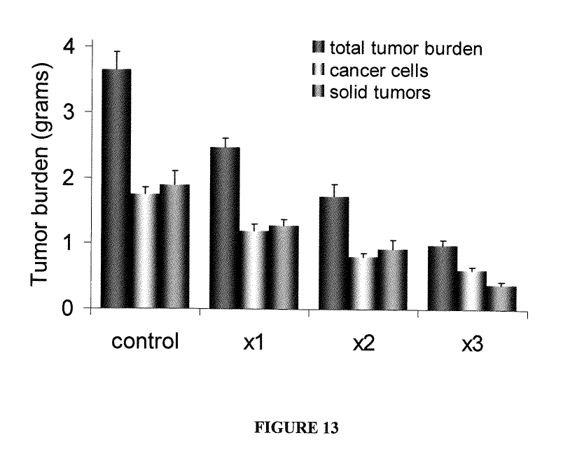

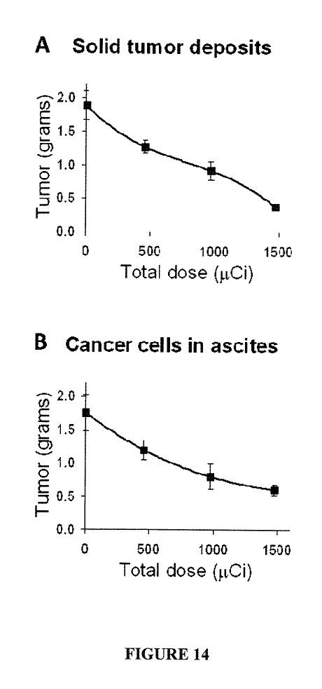

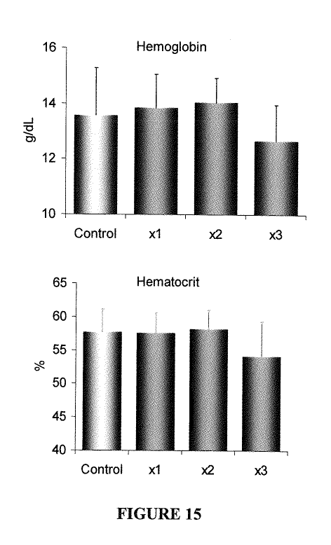

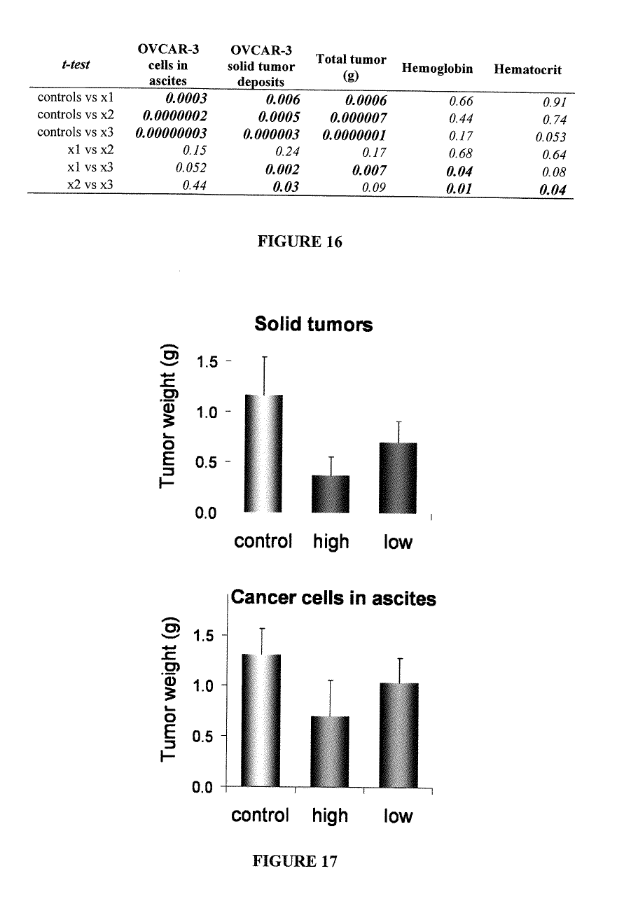

Female athymic NCr-nu/nu mice were received from NCI-Frederic (MD, USA). Identification transponders were implanted SQ in mice acclimated for one week. When mice reached the age of 8-weeks, all mice received IP implant of 5.6×10⁸ OVCAR-3 cells/mouse in 0.5 mL media without serum. This cancer cell load is double the size of implant described in Example 6 and allows for a rapid development of the advanced stages of the tumor. One weeks later mice were separated into three groups via a lottery as follows: mice in the control group were left untreated; mice the vehicle group received 0.3 mL vehicle (PBS containing 0.5% albumin and 5% DMSO); the remaining mice in groups ×1, ×2, and ×3 received an IP injection of 8b-slow in 0.3 mL vehicle; two weeks later mice in groups ×2 and ×3 received an IP injection of 8b-slow in 0.3 mL vehicle; and four weeks after the first dose mice in group ×3 received an IP injection of 8b-slow in 0.3 mL vehicle. Using the same schedule, mice in the vehicle group were given IP injections of 0.3 mL vehicle. The average total administered doses were as follows: group ×1:0.5 mCi (17.5 MBq); group ×2:1 mCi (37 MBq), and group ×3:1.5 mCi (55.5 MBq). All mice were killed six weeks after the administration of the first dose. FIG. 13 shows the weights of tumors extirpated from these mice and collected in the peritoneal lavage. FIG. 14 shows a unambiguous dose-response relationship between the tumor size and the total administered doses of 8b slow. The hemoglobin and hematocrit data shown in FIG. 15 indicates that these doses of radioactivity do not produce any significant hematological adverse effects. The data analyses confirmed that the observed differences are statistically significant. The table shown in FIG. 16 summarizes P values for all treatment groups.

### EXAMPLE 8

### Dose-Response Therapy in Early State OVCAR-3 Tumors

Female athymic NCr-nu/nu mice were received from NCI-Frederick (MD, USA) and were allowed to acclimate in Applicants' facilities until the age of 8 weeks. After this period of acclimation, 2.5×10⁸ OVCAR-3 cells were implanted into 36 mice. Four days later, each mouse received SQ transponders (micro-identification chips) and mice were divided into three groups at random: control mice receiving sham IP injections of 0.6 mL vehicle (n=12); mice in the high dose group were treated with one IP dose of 0.7 mCi (26 MBq) 8b slow in 0.6 mL vehicle (n=12); and mice the low dose group were treated with one IP dose of 0.29 mCi (10.7 MBq) 8b slow in 0.6 mL vehicle (n=12).

Five weeks after treatment, all mice were sacrificed and full necropsy was performed. A single dose of 8b slow given at the early stages of the disease reduces the tumor burden by >230%. The response of solid tumors seems better than the cells in ascites, >300% reduction in the weight of the solid tumor deposits (FIG. 17). The uptake and retention of radioactivity was measured in several tissues and in tumor. The radioactivity levels in blood are ˜30 times lower than in the tumor cells in ascites and ˜10 times lower than in the solid tumors. The hemoglobin and hematocrit levels indicated that the doses used in this therapy were not causing noticeable adverse effects. It is also encouraging that the liver uptake was low, <0.003% ID/g, indicating that the IP administration bypasses some of the hepatic metabolism of this compound.

### EXAMPLE 9

### Uptake Kinetics of Compound 7b in LS174T Human Colorectal Cancer Cells and in OVCAR-3 Human Ovarian Cancer Cells

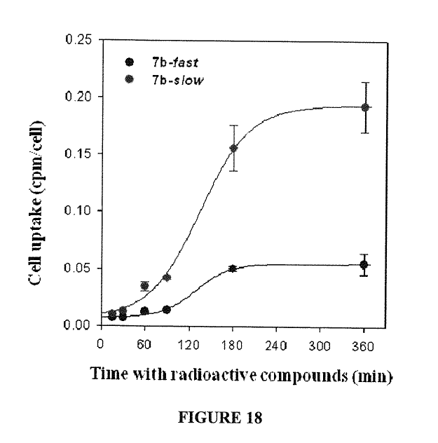

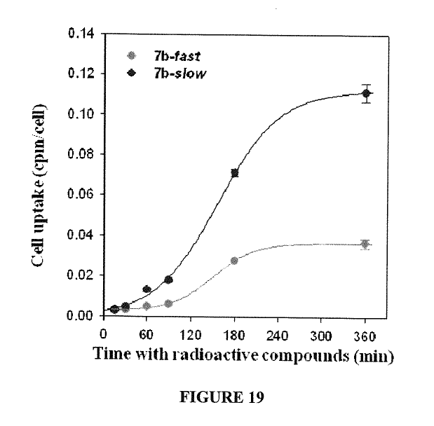

For each compound tested, LS174T cancer cells were plated in four 6-well plates at 2×10⁵ cells/well in 3 mL growth medium. After 24 h in culture, radioactive compounds were added to wells at predetermined times. The cells were incubated with compounds up to 360 min. Each point in time was tested in triplicate. Aliquots of media (0.5 mL) were removed from each well for gamma counting to determine the radioactive concentration in each well. At the end of incubation, the radioactive medium was aspirated and disposed. Cells were washed twice with 3 mL ice-cold PBS. Aliquots of wash PBS (0.5 mL) were also taken for gamma counting. Cells were trypsinized with 1.5 mL trypsin/EDTA and 1-mL aliquot of the cell suspension was counted in a gamma counter. FIGS. 18 and 19 demonstrate the uptake kinetics of 7b fast and 7b slow in OVCAR-3 ovarian cancer cells and LS174T colorectal cancer cells, respectively. The data indicates that the uptake is governed by the levels of BChE expression.

### EXAMPLE 10

### Clonogenic Assay Utilizing Compound 6b in Human Colorectal Adenocarcinoma Cells LS174T

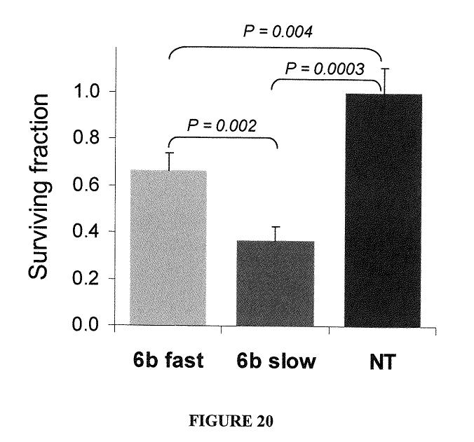

LS174T cells, 2×10⁶ cells/flask, were plated in T-75 flasks. After ˜18 hours in culture, the growth medium was removed from all flasks and replaced with either 15 mL fresh medium containing 6b fast (114.5±0.17 kBq/mL; 3.09±0.004 μCi/mL) or 15 mL fresh medium containing 6b slow (114.4±0.35 kBq/mL; 3.09±0.010 μCi/mL). Control cells were given 15 mL fresh nonradioactive medium. Triplicate 0.1-mL aliquots of medium were withdrawn from each flask and counted in a gamma counter to determine the concentration of radiolabeled compounds. After 4 hours in the incubator at 37° C., the growth medium was removed from all flasks. Monolayers were washed once with medium and 15 mL fresh medium was added to each flask. Cells were allowed to grow undisturbed for 24 hours at which time cells were trypsinized, counted, and their viability was determined. Cells were re-plated in T-25 flasks at plating densities of 500 cells/flask and 200 cells/flask. Each cell density was tested in quadruplicate. Seventeen to 21 days later, colonies were washed with 5 mL ice-cold PBS, followed by 5 mL PBS/methanol (1:1; v/v), and fixed in 5 mL methanol for 10 min. Methanol was discarded and flasks were left open to dry for a few hours. Crystal violet (5 mL; 0.25% in 1:1 PBS/methanol) was added to each flask to stain cells. After approximately 10 min, the dye was removed and flasks were rinsed first with tap water followed by distilled water, and were left to dry. Colonies were counted manually by two observers using the Wheaton colony counter (Wheaton, Millville, N.J., USA). FIG. 20 shows the surviving fractions calculated as the ratio of the number of colonies derived from cells treated with the radioactive compounds to the number of colonies derived from untreated control cells.

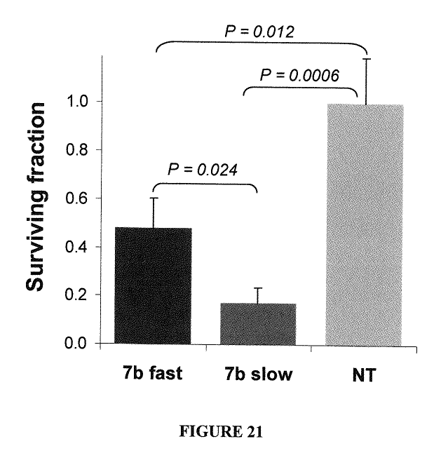

An alternative procedure with a higher concentration of the radioactive compounds and a shorter exposure time to the radioactive compounds was also employed. LS174T cells (2×10⁶) were plated in T75 flasks and allowed to grow for ˜18 hours, at which time the medium was removed and replaced with the medium containing radioactive compounds 7 fast and 7 slow at 5 μCi/mL concentration. Cells in control flasks received non-radioactive media. Triplicate 0.1-mL aliquots were taken from each flask for gamma counting. Cells were returned to the incubator for 4 hours. The medium was removed from all flasks, including controls, and the cell monolayer was washed once with fresh non-radioactive medium without FBS. Five mL 2.5% trypsin-EDTA was added each flask to dissociate the monolayer. Fresh FBS-containing medium was added to stop the action of trypsin and to form a single cell suspension. Cell numbers and cell viability were determined. All cell suspensions were centrifuged at 800 rpm for 10 min at 4° C. FBS-containing medium was added to cell pellets to produce 1×10⁶ cells/mL suspension. One mL of each suspension was counted in a gamma counter to determine cell-associated radioactivity. Cells were plated in duplicate T25 flasks at densities of 1,000 cells/flask, 500 cells/flask, and 100 cells/flask. The media was changed approximately once a week. After three weeks, colonies were processed as described above (FIG. 21). The cytotoxicity of these drugs appears to be BChE-dependent and it parallels the DNA uptake and retention.

### EXAMPLE 11

### Distribution of Radiolabeled Compounds in OVCAR-3 Cells Determined by Subcellular Fractionation

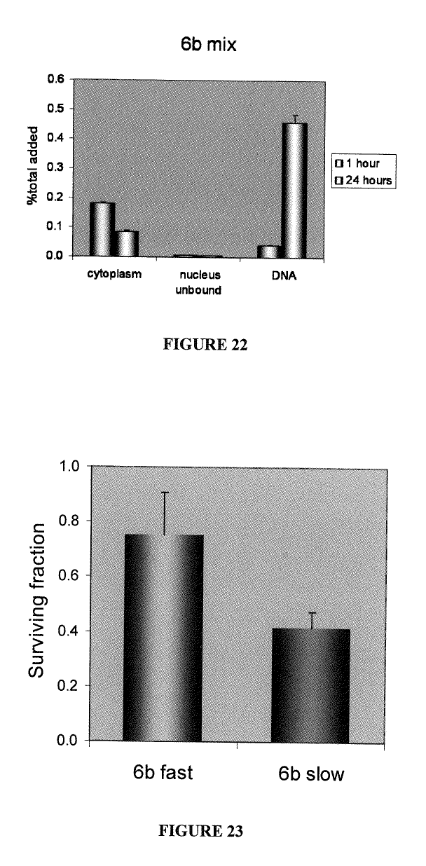

OVCAR-3 cells were plated into six flasks and allowed to attach overnight. The growth medium was removed and replaced with 10 mL fresh medium containing radioactive compounds. Cells were exposed to the compound for 1 hour after which time the radioactive medium was removed and replaced with 12 mL fresh medium. Aliquots of all radioactive growth media were counted in the gamma counter. Cells in three flasks were processed immediately. The cells in the remaining three flasks were cultured for 24 hours and then processed. The cell monolayer was rinsed with 5 mL PBS; trypsinized, cell were counted and their viability determined. Using NE-PER nuclear and cytoplasmic extraction reagents, cell content was fractionated and counted in a gamma counter to determine the compound distribution in various compartments of the cancer cell. FIG. 22 shows the subcellular distribution of 6b in OVCAR-3 cancer cells. This example illustrates how the selection of either fast or slow radiolabeled compounds can be tailored to the specific rate of cancer cell proliferation.

### EXAMPLE 12

### Cell Survival of U-87 Human Glioblastoma After Treatment with 6b

U-87 MG cell line is an epithelial cell line derived from a grade IV glioblastoma resected from the brain of a 44 years old, female Caucasian patient. U-87 cells were plated in four flasks for each isomer and allowed to attach for 24 h. The compounds 6b fast and 6b slow were added to U-87 cells at 1 μCi/mL (37 kBq/mL). Cells were incubated with the radioactive compounds for 24 h and the radioactive medium was removed. Cells were washed twice with fresh, nonradioactive medium and trypsinized. After appropriate dilutions in full growth medium, cells were re-plated in fresh medium (n=4 per compound). After 96 hours in culture, cells were harvested and their numbers counted using a Cellometer® disposable cell counting chamber (Nexcelom Bioscience, Lawrence, Mass.). Four flasks used as controls were sham-treated with a volume of PBS used to dissolve radioactive compounds. The control flasks were processed as described for the radioactive compound-treated cells. FIG. 23 demonstrates the surviving fractions of cells treated with both isomers of 6b. The surviving fraction is calculated as the ratio of cell number recovered from flasks treated with radioactive compounds to the cell number recovered from the control flasks. Averages and standard deviations are shown. The cell survival was also measured in a 96-well and 6-well formats.

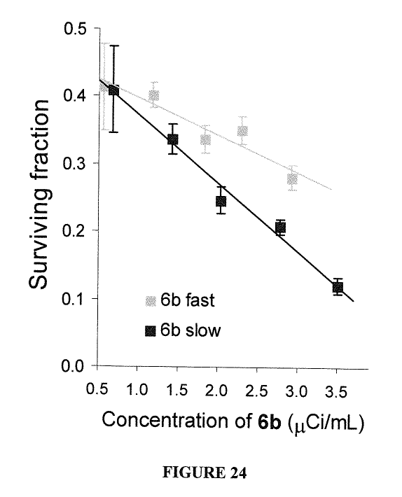

U-87 cells were plated in 6-well plates from suspension. The compounds 6b fast and 6b slow were added with media on day 0. Twenty-four hours later, radioactive medium was removed and fresh, full growth medium was added. Cells were grown for 72 h, medium was replaced and cells continued to grow for additional 72 h. Growth medium was removed; cell monolayers were washed with ice-cold PBS, followed by 1:1 (v/v) PBS-methanol. Cells were fixed with methanol for ˜10 min and plates were dried overnight. The cells were stained with 0.25% crystal violet for 10 min, rinsed with tap water followed by distilled water. Cells were dried at room temperature for ˜24 h. The stained cells were solubilized in 3.5 mM of an aqueous SDS solution and ethanol (SDS added first; followed by an equal volume ethanol). The OD was read at 560 nm. FIG. 23 illustrates a typical result of the present experiment. From FIG. 24, it can be deduced that when the concentration of either isomer of 6b is increased, the surviving fraction decreases at a constant rate. Moreover, data in FIG. 24 suggests that 6b slow is more cytotoxic to U-87 cells compared to the 6b fast isomer.

### EXAMPLE 13

### DNA Uptake of Compound 6b in U-87 Human Glioblastoma and the Subcellular

Distribution of Radioactivity Therein

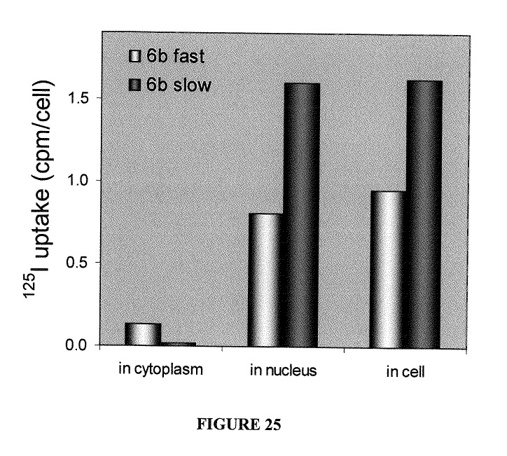

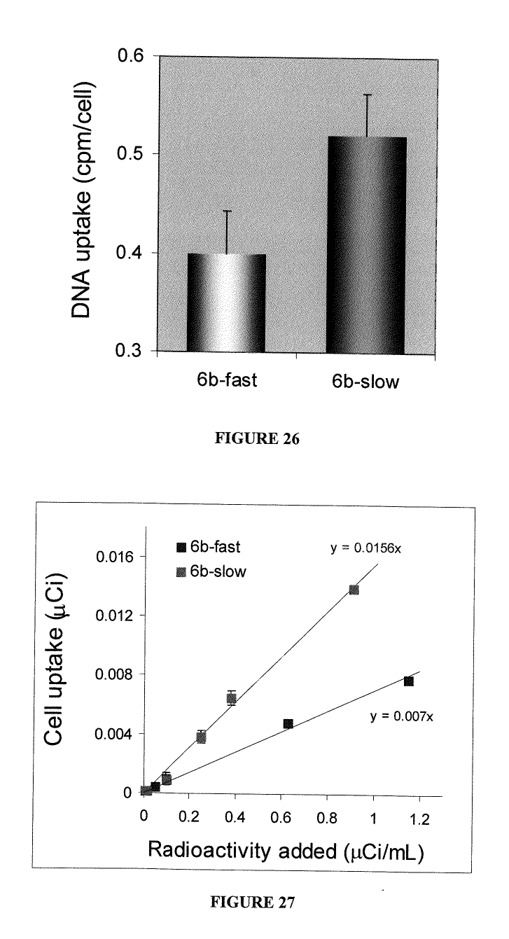

U-87 cells were plated in T75 flasks and allowed to attach for 24 h. Compounds 6b-fast and 6b slow were added to cells at 1 μCi/mL (37 kBq/mL) and 5 μCi/mL (185 kBq/mL). Cells were incubated with radioactive compounds from 24 h to 120 h. The radioactive medium was removed. Cells were washed twice with fresh, nonradioactive medium. Cells were trypsinized and their numbers and radioactive content determined. A similar study was also conducted in 96- and 24-well formats, which allowed more rapid analyses of several concentrations of 6b. Harvested cells were processed using NE-PER nuclear and cytoplasmic extraction reagents (Thermo Fisher Scientific, Rockford, Ill.). FIG. 25 shows the uptake and subcellular distribution of both diastereomers of 6b expressed in terms of the ¹²⁵I radioactivity (cpm/cell) in cytoplasm, in nucleus (i.e., DNA), and the total radioactivity in cell. U-87 cells plated in T75 flasks and allowed to grow for 24 h. Cells were treated with 0.75 μCi/mL 6b fast and 6b slow for 40 h at which time the radioactive medium was removed and replaced with fresh media. Cells were grown in fresh medium for additional 24 h and 72 h. Cells were trypsinized and DNA was extracted using the Qiagen method (Qiagen Genomic-tip 20/G; Qiagen, Valencia, Calif.). FIG. 26 shows the DNA content expressed in cpm/cell. The experiment demonstrates that following the uptake of the diastereomers into the cell, both isomers preferentially reside in the nucleus. Moreover, 6b slow demonstrates a greater overall cellular and nuclear uptake when compared to 6b fast.

### EXAMPLE 14

### Concentration-Dependent Uptake of Compound 6b by U-87 Human Glioblastoma Cells

U-87 cells were plated in 96-well plates and allowed to attach for ˜48 hours. The used medium was replaced with 6b-containing medium and the cells were grown in the presence of radioactivity for 40 h. The radioactive media was removed after 40 h of exposure. Aliquots (0.05 mL) of the radioactive medium were counted in a gamma counter. Cell monolayers were washed with PBS, PBS-methanol (2 min), and fixed in methanol (10 min). Fixed cells were allowed to dry and were stained with 0.25% crystal violet (10 min). To each well 0.1 mL ethanol was added (2 h) followed by 0.1 mL 3.5 mM SDS in water. OD at 560 nm was read. Solubilized cells were transferred into gamma counter tubes to determine their radioactive content. The plate was divided into individual wells and these were also added to the gamma counter tubes. FIG. 27 illustrates the concentration-dependent uptake. The experiment demonstrates that of the two diastereomers of 6b, 6b slow displayed almost a two-fold greater uptake by U-87 human glioblastoma cells compared to 6b fast.

### EXAMPLE 15

### Clonogenic Assay and Measurement of 6b Cellular Uptake in U-87 Human Glioblastoma Cells

U-87 cells were plated for 24 h before treatment and then treated for 24 h with concentrations of 6b fast and 6b slow ranging from 0.5 μCi/mL to 5 μCi/mL (18.5 to 185 kBq/mL). Cells were harvested, washed, and their numbers counted using a Cellometer® cell counter. Cells were diluted in fresh medium. Cells from each concentration of 6b fast and 6b slow were plated in three T25 flasks at two densities, 100 cells/flask and 500 cells/flask. Control cells were sham-treated with PBS and processed in a manner identical to cells treated with 6b. Cells were periodically examined and fresh medium was added every 5-7 days. Three weeks after plating, media was removed from the control flasks, cells were washed with 5 mL ice-cold PBS, followed by 5 mL PBS/methanol (1:1; v/v), and fixed in 5 mL methanol for 10 minutes. The methanol was discarded and flasks were left open to dry for a few hours. Crystal violet (5 mL; 0.25% in 1:1 PBS/methanol) was added to each flask to stain the cells. After approximately 10 min, the dye was removed and flasks were rinsed first with tap water followed by distilled water and were left to dry. The 6b-treated cells were monitored for additional 4 weeks during which time colonies were not formed in either 100 cells/flask or 500 cells/flask plating densities. This finding indicates the extraordinarily effective killing of U-87 cells by 6b.

### EXAMPLE 16

### Synthesis of Additional cycloSaligenyl Monophosphate Analogs and Diphosphate Analogs of 5-[¹²⁵I]-iodo-2′-deoxyuridine (cycloSal-[¹²⁵I]IUdRMP) and 5-[¹²⁵I]iodo-3′-fluoro-2′,3′-dideoxyuridine (cycloSal-[¹²⁵I]FIUdRMP)

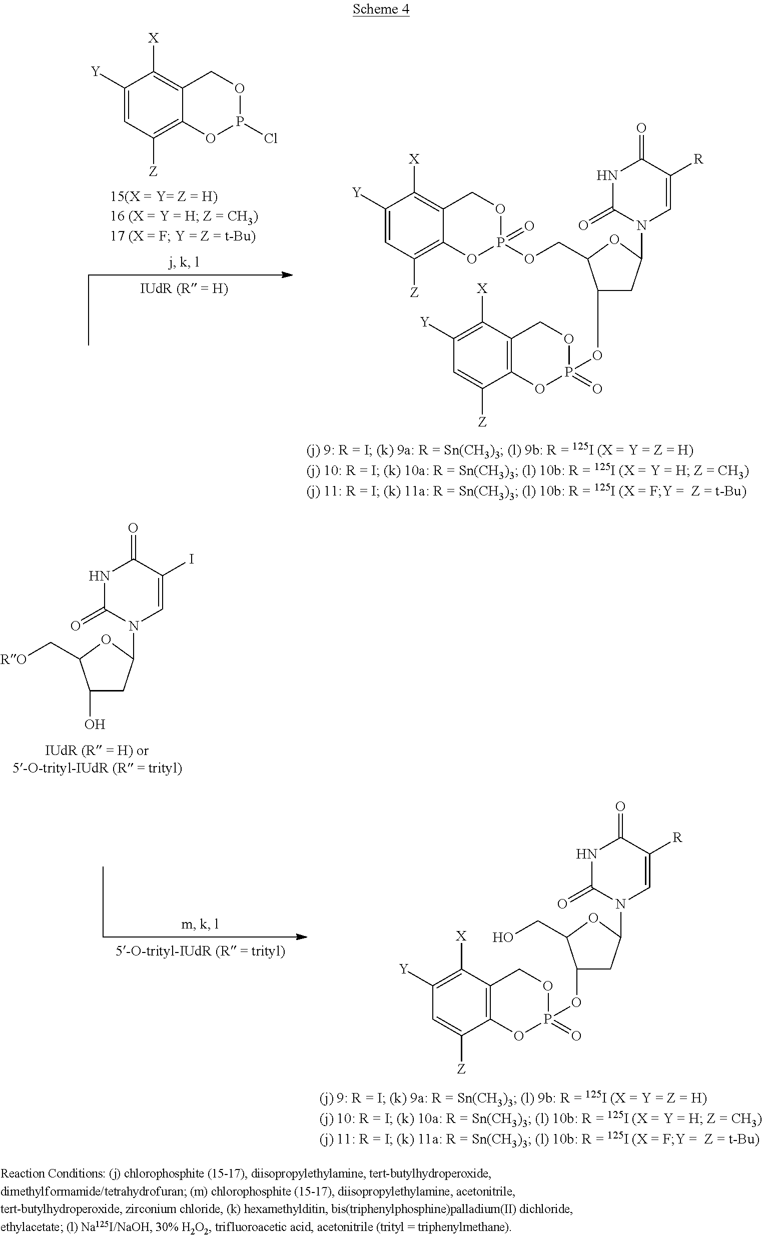

### Synthesis of 5-Iodo-3′-O-cycloSaligenyl-2′-deoxyuridine Monophosphate (12)

This compound was obtained in two ways: (1) as the side product (611 mg, 37% yield) in the preparation of 6 using Method II, or (2) by General Procedure C conducted with 5′-O-trityl IUdR (3.52 g, 5.90 mmol), DIPEA (1.8 mL, 1.33 g, 10.3 mmol) and crude chlorophosphite 15 (1.3 g, ˜8 mmol). Both synthetic pathways furnished 12 with the identical analytical data. General Procedure C was performed for 45 min in MeCN (30 mL), with the subsequent oxidation using a solution of t-BuOOH (2.2 mL, ≧11 mmol). After workup, the crude solid residue dissolved in MeCN (34 mL) was treated with ZrCl₄ (1.65 g, 7.0 mmol) and gave 12 (1.21 g, 39%) as colorless rigid foam; R_{f} value 0.58 (DCM/MeOH, 10:0.7). The HPLC analysis has shown a diastereomeric mixture (44:56 ratio): 12 fast, t_{R}=37.9 min and 12 slow, t_{R}=38.6 min (≧98% pure, UV at 280 nm); column: ACE C18, 100 Å (5 μm, 4.6×250 mm); eluent: solvent A 10% MeCN in water, solvent B MeCN; eluted at 0.8 mL/min with a linear gradient of B from 0-25% over 40 min, and a gradient of B from 25-95% B for 20 min. ¹HNMR (DMSO-d₆) δ=10.61 (s, 1H, NH), 10.58 (s, 1H NH), 8.36 (s, 1H, uridine-H6), 8.34 (s, 1H, uridine-H6), 7.13 (m, 1H, aryl-H4), 7.06-6.98 (m, 3H, aryl-H3, aryl-H5, aryl-H6), 6.21 (t, 1H, H1′, J=7.0 Hz), 5.97 (t, 1H, H1′, J=7.0 Hz), 5.34-5.28 (m, 2H, benzyl), 4.24-4.22 (m, 1H, H3′), 3.82-3.76 (m, 2H, H4′, H5′), 3.94-3.89 (m, 1H, H5″), 3.55 (bd, C5′-OH, J=4.0 Hz), 2.56-2.44 (m, 1H, H2′), 2.34-2.26 (m, 1H, H2″) ppm. ³¹P NMR (DMSO-d₆) δ=−9.74, −9.77 ppm. MSFAB-HR (m/z): [M+Li]⁺calcd for C₁₆H₁₆N₂O₈PILi, 528.9849, found 528.9844 The ¹³C isotope was observed at 529.9893 within 1.9 ppm of expected 529.9883.

### Synthesis of 5-Iodo-3′-O-cyclo(3-methylSaligenyl)-2′-deoxyuridine Monophosphate (13)

Compound 13 was obtained in two ways: (1) as the side product (1.17 g, 31%) during the preparation of 7 using Method II, or (2) by General Procedure C conducted with 5′-O-trityl IUdR (2.34 g, 3.93 mmol), DIPEA (1.5 mL, 1.11 g, 8.6 mmol) and crude chlorophosphite 16 (1.1 g, ˜6 mmol). Isolated product 13 in both synthetic pathways, showed the identical analytical data. General Procedure C was carried out in 20 mL of MeCN for 60 min, and the oxidation with a solution of t-BuOOH (1.6 mL, ≧8 mmol) subsequent to the phosphitylation. After workup, the crude solid dissolved in MeCN (40 mL) was treated with ZrCl₄ (1.0 g, 4.3 mmol) and gave 13 (842 mg, 40%) as colorless rigid foam; R_{f} value 0.64 (DCM/MeOH, 10:0.7). Further HPLC purification (23 mg of 13, ˜4 mg per injection) was performed on Columbus C18, 100 Å (5 μm, 10×250 mm) column; eluent: solvent A 20% MeCN in water and solvent B MeCN; eluted at 2.5 mL/min with a linear gradient of B from 0-65% over 35 min. Product 13 (11.9 mg, ≧98% pure, UV at 280 nm) was collected within 28-30 min after the injection. The HPLC analysis showed a mixture of diastereomers: 13 fast, t_{R}=21.8 min; 13 slow, t_{R}=22.4 min (≧98% pure, UV at 280 nm); column: ACE C18, 100 Å (5 μm, 4.6×250 mm); eluent: solvent A 20% MeCN in water, solvent B MeCN; eluted at 1 mL/min with a linear gradient of B from 0-60% over 45 min, and the using a linear gradient of B from 45-95% B for 15 min. ¹HNMR (DMSO-d₆) δ=10.82 (s, 1H, NH), 10.65 (s, 1H NH), 8.31 (s, 1H, uridine-H6), 8.28 (s, 1H, uridine-H6), 7.23-7.06 (m, 3H, aryl-H4, aryl-H5, aryl-H6) 6.20-6.17 (m, 1H, H1′), 5.33-5.27 (m, 2H, benzyl), 4.30-4.23 (m, 1H, H3′), 3.83-3.76 (m, 2H, H4′, H5′), 3.94-3.89 (m, 1H, H5″), 3.61 (bd, C5′-OH, J=4.0 Hz), 2.56-2.44 (m, 1H, H2′), 2.34-2.26 (m, 4H, H2″, aryl-C3-CH₃) ppm. ³¹P NMR (DMSO-d₆) δ=−9.34, −9.38 ppm. MSFAB-HR (m/z): [M+H]⁺calcd for C₁₇H₁₉N₂O₈PI, 536.9924, found 536.9940. The ¹³C isotope peak was measured at 537.9981; 4.3 ppm of the expected value.

### Synthesis of 5-Iodo-3′-O-[cyclo-3,5-di-(tert-butyl)-6-fluoroSaligenyl]-2′-deoxyuridine Monophosphate (14)

Compound 14 was obtained in two ways: (1) as the side product (3.31 g 36% yield) during the preparation of 8 using Method II, or (2) by conducting General Procedure C with 5′-O-trityl IUdR (3.52 g, 5.90 mmol), DIPEA (2 mL, 1.48 g, 11.5 mmol) and crude chlorophosphite 17 (2.6 g, ˜8 mmol). Isolated products 14 from both synthetic pathways showed the identical analytical data. General Procedure C was carried on for 90 min in 30 mL of MeCN and the oxidation with a solution of t-BuOOH (1.5 mL, ≧6 mmol) was following the phosphitylation. After the workup, the crude solid dissolved in MeCN (50 mL) was treated with ZrCl₄ (1.72 g, 7.38 mmol) and gave 14 (1.65 g, 43%) as colorless rigid foam (93% pure by HPLC analysis); R_{f} value 0.66 (DCM/MeOH, 10:0.7). Further HPLC purification (31 mg of 14, ˜5 mg per injection) was performed on Columbus C18, 100 Å (5 μm, 10×250 mm) column; eluent: solvent A 50% MeCN in water, solvent B MeCN; eluted at 2.5 mL/min with a linear gradient of B from 0-40% over 15 min, and 40% B (isocratic) for 30 min. Product 14 (18.2 mg, ≧98% pure, UV at 280 nm) was collected within 25-27 min after the injection. The HPLC analysis showed a mixture of diastereomers: 14 fast, t_{R}=31.1 min and 14 slow, t_{R}=33.3 min; column: Columbus C8, 100 Å (5 μm, 4.6×250 mm); eluent: solvent A 50% MeCN in water, solvent B MeCN; eluted at 1 mL/min with A for 45 min (isocratic) and then a linear gradient of B from 0-95% B over 5 min, and 95% B for 10 min. ¹HNMR (DMSO-d₆) δ=10.89 (s, 1H, NH), 10.84 (s, 1H NH), 7.89 (s, 1H, uridine-H6), 7.84 (s, 1H, uridine-H6), 7.29-7.24 (m, 1H, aryl-H4), 6.27-6.22 (m, 1H, H1′), 5.56-5.44 (m, 2H, benzyl), 4.35-4.22 (m, 1H, H3′), 4.19-4.15 (m, 1H, H4′), 3.43 (bd, C5′-OH, J=4.0 Hz), 2.54-2.46 (m, 2H, H5′, H5″), 2.27-2.23 (m, 1H, H2′), 2.15-2.11 (m, 1H, H2″), 1.39, 1.35, 1.32, 1.31 (overlapped s, 18H, aryl-C3 -3 ×CH₃t-Bu and aryl-C5-3×CH₃-t-Bu) ppm. ³¹P NMR (DMSO-d₆) δ=−8.90, −8.77 ppm. MSFAB-HR (m/z): [M+H]⁺calcd for C₂₄H₃₂N₂O₈PFI, 653.0925, found 653.0930. The ¹³C isotope peak measured 654.0845; −2.0 ppm of the expected value.

### Synthesis of 5-Trimethylstannyl-3′-O-cyckSaligenyl-2′-deoxyuridine Monophosphate (12a)

General Procedure D was carried out with 5-iodo-3′-O-cycksaligenyl-2′-deoxyuridine monophosphate 12 (193 mg, 0.37 mmol), hexamethylditin (157 mg, 0.48 mmol) and the palladium(II) catalyst (26 mg, 0.037 mmol) in EtOAc (30 mL) until starting 12 despaired on TLC (˜1.5 h). The crude product was initially separated and purified on a silica gel column (DCM/MeOH, 10:0.6). Further purification was done using the HPLC equipped with a semi preparative Columbus C18, 100 Å (5 μm, 10×250 mm) column; eluent: solvent A 45% MeCN, solvent B MeCN; eluted at 2.5 mL/min with a linear gradient of B for 60 min. Combined fractions, after repetitive evaporation from dried MeCN, gave pure stannane 12a (119 mg, 57%) as colorless amorphous solid; R_{f} value 0.64 (DCM/MeOH, 10:0.3). The HPLC analysis showed a mixture of diastereomers: 12a fast, t_{R}=47.0 min and 12a slow, t_{R}=47.4 min (≧96% pure, UV at 280 nm); column: ACE C18, 100 Å (5 μm, 4.6×250 mm); eluent: solvent A 10% MeCN in water, solvent B MeCN; eluted at 1 mL/min with a linear gradient of B from 0-95% over 45 min, and then 95% B for 15 min. 1HNMR (CDCl₃) δ=8.79 (s, 1H, NH), 8.63 (s, 1H NH), 7.49 (s, 1H, uridine-H6, ³J_{H,Sn}=8.5 Hz), 7.37 (s, 1H, uridine-H6, ³J_{H,Sn}=18.5 Hz), 7.17 (t, 1H, aryl-H4, J=7.6 Hz), 7.14-7.07 (m, 3H, aryl-H3, H5, H6), 6.23-6.16 (m, 1H, H1′), 5.42-5.36 (m, 2H, benzyl), 4.31-4.28 (m, 1H, H3′), 4.25-4.21 (m, 1H, H4′), 3.97-3.85 (m, 2H, H5′, H5″, H4′), 3.58 (bd, 1H, C5′-OH, exchangeable with D₂O, J=4.4 Hz), 2.61-2.45 (m, 2H, H2′, H2″), 0.32 (t, 3×3H, 3×CH₃Sn, ²J_{H,Sn}=29.0 Hz) ppm. ³¹P NMR (CDCl₃) δ=−9.80, −9.82 ppm. ¹¹⁹Sn NMR (CDCl₃) δ=−1.47 ppm. MSFAB-HR (m/z): [M+Li]⁺calcd for C₁₉H₂₅N₂O₈PSnLi, 567.0531, found 567.0549. The ¹³C isotope measured 568.0559; −0.9 ppm of the expected 568.0565 value.

### Synthesis of 5-Trimethylstannyl-3′-O-cyclo(3-methylSaligenyl)-2′-deoxyuridine Monophosphate (13a)

General Procedure D was carried out with iodo-3′-O-cyclo(3-methylsaligenyl)-2′-deoxyuridine Monophosphate 13 (241 mg, 0.45 mmol), hexamethylditin (192 mg, 0.58 mmol) and the palladium(II) catalyst (32 mg, 0.046 mmol) in EtOAc (30 mL) until starting 13 despaired on TLC (˜2 h). The crude product was separated and purified at first on a silica gel column (DCM/MeOH gradient, 10:0.6-0.9). Final purification proceeded on the HPLC equipped with a semi preparative Columbus C18, 100 Å (5 μm, 10×250 mm) column; eluent: solvent A 45% MeCN, solvent B MeCN; eluted at 2.5 mL/min with a linear gradient of B for 60 min. Combined and dried fractions, gave pure stannane 13a (139 mg, 54%) as colorless amorphous solid; R_{f} value 0.71 (DCM/MeOH, 10:0.8). The HPLC analysis showed a mixture of diastereomers: 13a fast, t_{R}=38.5 min and 13a slow, t_{R}=39.7 min (≧ 98% pure, UV at 280 nm); column: ACE C18, 100 Å (5 μm, 4.6×250 mm); eluent: solvent A 15% MeCN in water, solvent B MeCN; column eluted at 1 mL/min with a linear gradient of B from 0-25% over 50 min, then a gradient of B from 25-95% B over 5 min, and the 95% for 5 min. ¹HNMR (CDCl₃) δ=8.73 (bs, 1H, NH), 7.52 (s, 1H, uridine-H6, ³J_{H,Sn}=18.6 Hz), 7.43 (s, 1H, uridine-H6, ³J_{H,Sn}=18.6 Hz), 7.18-7.03 (m, 3H, aryl-H4, H5, H6), 6.27-6.18 (m, 1H, H1′), 5.46-5.38 (m, 2H, benzyl), 4.30-4.25 (m, 1H, H3′), 4.23-4.21 (m, 1H, H4′), 3.97-3.85 (m, 2H, H5′, H5″), 3.63 (bd, 1H, C5′-OH, exchangeable with D₂O, J=4.3 Hz), 2.64-2.43 (m, 5H, H2′, H2″, aryl-C3-CH₃), 0.37 (t, 3×3H, 3×CH₃Sn, ²J_{H,Sn}=29.0 Hz) ppm. ³¹P NMR (CDCl₃) δ=−9.84, −9.77 ppm. ¹¹⁹Sn NMR (CDCl₃) δ=−1.41 ppm. MSFAB-HR (m/z): [M+H]⁺calcd for C₂₀H₂₈N₂O8PSn, 575.0605, found 575.0624. The ¹³C isotope measured 576.0624; −2.5 ppm of the expected value.

### 5-Trimethylstannyl-3′-O -(cyclo-3,5-di-(tert-butyl)-6-fluoroSaligenyl)-2′-deoxyuridine Monophosphate (14a)

General Procedure D was carried out with 5-iodo-3′-O-(cyclo-3,5-di-(tert-butyl)-6-fluorosaligenyl)-2′-deoxyuridine monophosphate 14 (306 mg, 0.47 mmol), hexamethylditin (205 mg, 0.62 mmol) and the palladium(II) catalyst (34 mg, 0.048 mmol) in EtOAc (40 mL) until starting 14 despaired on TLC (˜3 h). The crude product was separated on a silica gel column (DCM/MeOH, 10:0.4) and the purification repeated (using EtOAc/hexanes, 10:5). Final purification was conducted using the HPLC equipped with a semi preparative Columbus C18, 100 Å (5 μm, 10×250 mm) column; eluent: solvent A 45% MeCN, solvent B MeCN and a column eluted at 2.5 mL/min with a linear gradient of B from 0-95% over the period of 60 min. Combined fractions were evaporated under vacuum and the residue dried by the repetitive evaporation from dry MeCN, gave pure stannane 14a (206 mg, 63%) as colorless ridged foam; R_{f} value 0.79 (DCM/MeOH, 10:0.5). The HPLC analysis showed a mixture of diastereomers: 14a fast, t_{R}=26.4 min and 14a slow, t_{R}=26.9 min (≧98% pure, UV at 220 and 280 nm); column: ACE C18, 100 Å (5 μm, 4.6×250 mm); eluent: solvent A 50% MeCN in water, solvent B MeCN; eluted at 1 mL/min with a linear gradient of B from 0-95% over 40 min, then 95% B for 20 min. ¹HNMR (CDCl₃) δ=8.63 (bs, 1H NH), 7.44 (s, 1H, uridine-H6, ³J_{H,Sn}=18.5 Hz), 7.40 (s, 1H, uridine-H6, ³J_{H,Sn}=18.5 Hz), 7.19 (t, 1H, aryl-H4, J=7.6 Hz), 6.20-6.14 (m, 1H, H1′), 5.44-5.32 (m, 2H, benzyl), 4.33-4.29 (m, 1H, H3′), 4.27-4.24 (m, 1H, H4′), 3.94-3.82 (m, 2H, H5′, H5″), 3.66 (bd, 1H, C5′-OH, exchangeable with D₂O, J=4.4 Hz), 2.64-2.47 (m, 2H, H2′, H2″), 1.325, 1.315, 1.312, 1.311 (overlapped s, 18H, aryl-C3-3×CH₃-t-Bu and aryl-C5-3×CH₃-t-Bu), 0.24 (t, 3×3H, 3×CH₃Sn, ²J_{H,Sn}=29.0 Hz) ppm. ³¹P NMR (CDCl₃) δ=−9.72, −9.62 ppm. ¹¹⁹Sn NMR (CDCl₃) δ=−1.43 ppm. MSFAB-HR (m/z): [M+H]⁺calcd for C₂₇H₄₁N₂O₈PFSn, 691.1607, found 691.1635. The ¹³C isotope measured 692.1648; 1.1 ppm of the expected value.

### Synthesis of 5-[¹²⁵O]-Iodo-3′-O-cycloSaligenyl-2′-deoxyuridine Monophosphate (12b)

Four consecutive radioiodinations were carried out according to General Procedure E within the 0.25-7.1 mCi range, to give overall 9.74 mCi of 12b. An average isolated yield of the product was 87%. The latest radiolabeling was performed with the diastereomeric stannane 12a (˜100 μg, ˜97% pure, UV at 280 nm) and [¹²⁵I]NaI/NaOH (65 μL, 7.1 mCi). The HPLC purification of the product proceeded on ACE C18, 100 Å (5 μm, 4.6×250 mm) column; eluent: solvent A 10% MeCN in water, solvent B MeCN; eluted at 1.0 mL/min with a linear gradient of B from 0-40% over 40 min, followed by a gradient of B from 40-95% for the period of 20 min. The radioactivity peak of 12b (6.32 mCi, 89%) was collected in four fractions, within 21-25 min after the injection of ˜500 μL (6.92 mCi) of the reaction mixture. An excess of the unreacted tin precursor 12a was separated from the radioiodinated product, eluting ˜5 min later (t_{R}=30.2 min). Combined fractions containing 12b were evaporated, reconstituted in MeCN (˜1.8 mCi/mL) and 10 μL volume (˜18 μCi) was re-injected on the HPLC: ACE C18, 100 Å (5 μm, 4.6×250 mm) column; eluent: solvent A 10% MeCN in water, solvent B MeCN; eluted at 0.8 mL/min with a linear gradient of B from 0-40% over 45 min, then a gradient of B from 40-95% for a period of 15 min. Analysis showed a mixture (44:56 ratio) of diastereomers: 12b fast, t_{R}=26.4 min and 12b slow, t_{R}=26.7 min (≧98% pure, Bioscan NaI(T)). Diastereomers of 12b were not separated preparatively. Co-injected solutions (in 50% MeCN in water) of the purified 12b and the corresponding nonradioactive analog 12, with a parallel monitoring of the radioactivity and UV signal, has shown the identical elution mobility of both analogs. Diastereomer 12b fast eluted at t_{R}=26.3 min and 12b slow at t_{R}=26.7 min, together with 12 fast; t_{R}=26.1 min and 12 slow; t_{R}=26.4 min.

### 5-[¹²⁵I]Iodo-3′-O-cyclo(3-methylSaligenyl)-2′-deoxyuridine Monophosphate (13b)

The total amount of prepared 13b was 12.5 mCi, obtained in five successive radioiodinations of 13a, carried out within 0.25-5.1 mCi range. Each reaction proceeded according to General Procedure E. An average isolated yield of the product was 73%. The latest radiolabeling was performed with the diastereomeric stannane 13a (˜100 μg) and [¹²⁵I]NaI/NaOH (45 μL, 4.71 mCi). The HPLC purification proceeded efficiently on Jupiter C18, 300 Å (5 μm, 4.6×250 mm) column; eluent: solvent A 10% MeCN in water, solvent B MeCN. A column was eluted at 1.0 mL/min with a linear gradient of B from 0-95% over 45 min, then 95% B for the period of 15 min. The radioactivity peak of 13b (3.35 mCi, 71%) was collected within three fractions (24-27 min) after the injection of ˜300μL (4.35 mCi) of the reaction mixture. An excess of the unreacted tin precursor 13a was separated from the radioiodinated product, eluting ˜6 min later (t_{R}=29.6 min). Combined fractions containing the purified 13b were evaporated, reconstituted in MeCN (˜1.2 mCi/mL) and 10 μL, volume (˜12 μCi), was re-injected on the HPLC column: Jupiter C18, 300 Å (5 μm, 4.6×250 mm); eluent: solvent A 10% MeCN in water, solvent B MeCN; eluted at 1.0 mL/min with a linear gradient of B from 0-95% over 45 min, then 95% B for the period of 15 min. The analysis showed a mixture (47:53 ratio) of diastereomers 13b fast, t_{R}=23.4 min and 13b slow, t_{R}=23.7 min (≧98% pure, Bioscan NaI(T)). Diastereomers of 13b were not separated preparatively. Co-injected solutions (in 50% MeCN) of the purified 13b (˜18 μCi, 15 μL) and the nonradioactive analog 13 (˜17 μg, 25 μL) were analyzed, with monitoring the radioactivity (Bioscan NaI(T)) and UV at 280 nm, on ACE C18, 100 Å (5 μm, 4.6×250 mm) column; eluent: solvent A 10% MeCN in water, solvent B MeCN; eluted at 1.0 mL/min with a linear gradient of B from 0-95% over a period of 60 min. The analysis confirmed the identical elution of both: [¹²⁵I]- and [¹²⁷I]-iodoanalogs.

### 5-[¹²⁵I]-Iodo-3′-O-(cyclo-3,5-di-(tert-butyl)-6-fluoroSaligenyl)-2′-deoxyuridine Monophosphate (14b)

General Procedure E was carried out within 0.5-5.7 mCi range, to give after four conducted radioiodinations, 12.6 mCi of 14b in an average yield of 86%. The latest radiolabeling proceeded with stannane 14a (˜110 μg) and [¹²⁵I]NaI/NaOH (60 μL, 5.71 mCi). The HPLC purification of the crude product was achieved on Jupiter C18, 300 Å (5 μm, 4.6×250 mm) column; eluent: solvent A 40% MeCN in water, solvent B MeCN. A column was eluted at 1.0 mL/min the flow rate, with a linear gradient of B from 0-95% over 35 min, followed by 95% B for the period of 25 min. The product (5.02 mCi, 88%) collected within 21-23 min after the injection of 425 μL (˜5.5 mCi) of the reaction mixture was separated from an excess of the unreacted tin precursor 14a, which eluted ˜10 min later (32.8-33.6 min). Appropriate fractions were combined, evaporated with a stream of nitrogen, and the residue further dried in high vacuum. The mixture of purified 14b (˜12 μCi, 10 μL) and its corresponding nonradioactive analog 14 (˜15 μg, 20 μL) in acetonitrile, was reinjected onto the HPLC, using the same settings as during the separation of the crude product. The analysis has shown 14b fast at t_{R}=21.2 min and 14b slow; t_{R}=21.7 min, co-eluting with diastereomers of the [¹²⁷I]-iodoanalog: 14 fast, t_{R}=20.9 min and 14 slow, t_{R}=21.6 min. Diastereomers of 14b were most efficiently separated on ACE C18, 100 Å (5 μm, 4.6×250 mm) column, eluted with 50% MeCN in water at the 0.8 mL/min flow rate. Each isomer, 14b fast (t_{R}=41.2 min) and 14b slow (t_{R}=45.7 min), was ≧98% pure (Bioscan NaI(T) detector). The complete separation of diastereomers in the single HPLC run was attained, if the total amount of 14b loaded onto a column was ≦320 μCi. Larger lots of single diastereomers were obtained in multiple injections.

The examples provided, above, indicate that compounds of formula (I) bind to a malignant tumor cell marker, BChE, access the nucleus, incorporate into the DNA of a tumor cell, and selectively deliver a radioisotopically labeled moiety that kills malignant tumors. Compounds of formula I can also be imaged in xenografted tumors in mice after delivery, thereby allowing for diagnostic studies of a cancer in vivo. Additionally, of the diastereomers of formula I that were tested, the slow eluting isomers are the more active therapeutics.

Several of the compounds described herein have exhibited activity for the treatment and diagnosis of Alzheimer's disease. See U.S. Patent Publication No. 2009/0117041, which is commonly owned with the present application.

A number of patent documents and non-patent documents are cited in the foregoing specification in order to describe the state of the art to which this invention pertains. The entire disclosure of each of the cited documents is incorporated by reference herein.

While various embodiments of the present invention have been described and/or exemplified above, numerous other embodiments will be apparent to those skilled in the art upon review of the foregoing disclosure. The present invention is, therefore, not limited to the particular embodiments described and/or exemplified, but is capable of considerable variation and modification without departure from the scope of the appended claims.

Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All resonant sensors and methods of use thereof that embody the present invention can, in alternate embodiments, be more specifically defined by any of the transitional terms “comprising”, “consisting essentially of” and “consisting of”.

1. A method of treatment of a tumor in a patient in need of such treatment, said tumor comprising cancer cells characterized by at least one of butyrylcholinesterase expression and androgen binding affinity, said method comprising administering to said patient a therapeutically effective amount of at least one compound of the formula: 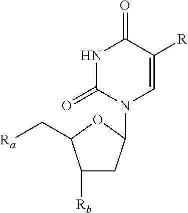
wherein Rₐ represents OH or: 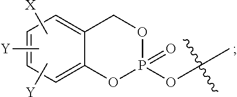
X represents H, F, Cl, or a C₁-C₈ alkyl, or C₁-C₈ alkoxy group;
Y represents H, C₁-C₈ alkyl, C₅-C₁₄ aryl, or a C₅-C₁₄ aryloxy group;
Z represents H, C₁-C₈ alkyl, C₅-C₁₄ aryl, or a C₅-C₁₄ aryloxy group;
R represents halogen, radiohalogen, or a C₁-C₈ alkyl, C₅-C₁₄ aryl, C₁-C₈ alkylthio, C₁-C₈ alkoxy, C2-C₆ alkenyl, C₂-C₆ alkynyl, C₃-C₁₂ cycloalkyl, or Sn(C₁-C₄ alkyl)₃ group;
R_{b} represents halogen, radiohalogen, or a C₁-C₆ alkoxy or C₁-C₈ alkanoate group, an androgen receptor binding ligand linked to the said compound via a cleavable linking moiety, or: 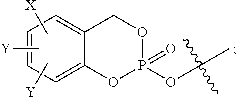
any of said alkyl, alkenyl, alkynyl, alkylthio, alkoxy and cycloalkyl group being optionally substituted by at least one halogen, OH, SH, NH₂, C₁-C₄ monoalkylamino, C₁-C₄ dialkylamino, COOH, CN, NO₂, alkyl, C₁-C₄ alkoxy or phenyl group, any of said aryl, aryloxy, and phenyl group being optionally substituted by at least one halogen, OH, SH, NH₂, C₁-C₄ monoalkylamino, C₁-C₄ dialkylamino, COOH, CN, NO₂, C₁-C₄ alkyl or C₁-C₄ alkoxy group; said radiohalogen represents ¹²³I, ¹²⁴I, ¹²⁵I, ¹³¹I, ²¹¹At, ¹⁸F, ⁷⁶Br, ⁷⁷Br, or ^{80m}Br; stereoisomeric forms and pharmaceutically acceptable salts of said at least one compound;
with the proviso that at least one of the Rₐ and R_{b} substitutents represents: 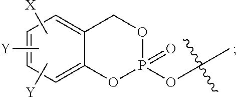
and
the wavy line indicating the point of attachment to the ribose moiety.

2. The method of claim 1, wherein said patient is administered at least one compound of the formula: 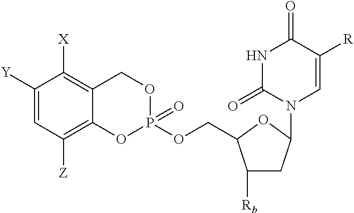
wherein X represents H, F, Cl, or a C₁-C₄ alkyl, or C₁-C₄ alkoxy group;
Y represents H or a C₁-C₄ alkyl group;
Z represents H or a C₁-C₄ alkyl group;
R represents halogen, radiohalogen, or a C₁-C₄ alkyl, C₁-C₄ alkoxy or phenyl group;
R_{b} represents halogen, radiohalogen, OH or a C₁-C₄ alkoxy group, or an androgen receptor binding ligand linked to the said compound via a cleavable linking moiety;
any of said alkyl, alkoxy and phenyl group being optionally substituted by at least one halogen, OH, SH, NH₂, C₁-C₄ monoalkylamino, C₁-C₄ dialkylamino, COOH, CN, NO₂, C₁-C₄ alkyl or C₁-C4 alkoxy; and said radiohalogen represents ¹²³I, ¹²⁴I, ¹²⁵I, ¹³¹I, ²¹¹At, ¹⁸F, ⁷⁶Br, ⁷⁷Br, or ^{80m}Br; and stereoisomeric forms and pharmaceutically acceptable salts of said at least one compound.

3. The method of claim 1, wherein the androgen receptor binding ligand is selected from the group consisting of an androgen receptor agonist and an androgen receptor antagonist.

4. The method of claim 3, wherein the androgen receptor agonist is selected from the group consisting of 4-dihydrotestosterone (DHT), testosterone, mibolerone, methyltrienolone, and methyltestosterone.

5. The method of claim 3, wherein the androgen receptor antagonist is selected from the group consisting of hydroxyflutamide, flutamide, cyproterone acetate, spironolactone, ketoconazole, and finasteride.

6. The method of claim 1, wherein said patient is administered a compound selected from the group consisting of:

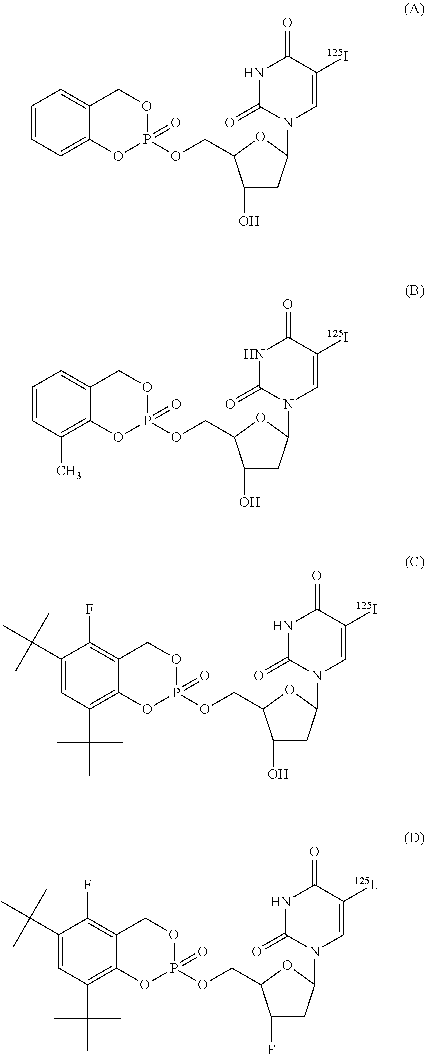

7. The method of claim 6, wherein said compound (A), (B), (C) or (D) is administered in the form of the fast isomer, the slow isomer, or a mixture thereof.

8. The method of claim 1, wherein said compound is administered in the form of the slow isomer thereof.

9. The method of claim 1, wherein the cancer cells are selected from the group consisting of ovarian, glioma, colorectal, breast, prostate, meningioma, head and neck, or pancreatic cancer cells.

10. The method of claim 1, wherein said at least one compound is administered by a method selected from the group consisting of intravenous, intraperitoneal, and intratumoral administration.

11. The method of claim 10, wherein said at least one compound is administered periodically for a term of years.

12. The method of claim 11, wherein said at least one compound is administered daily.

13. A method of diagnosing a patient for the presence of a tumor, said tumor comprising cancer cells characterized by at least one of butyrylcholinesterase expression and androgen binding affinity, said method comprising administering to said patient an effective amount for diagnostic imaging of a compound of the formula: 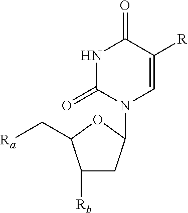
wherein Rₐ represents OH or: 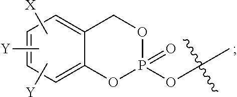
X represents H, F, Cl, or a C₁-C₈ alkyl, or C₁-C₈ alkoxy group;
Y represents H, C₁-C₈ alkyl, C₅-C₁₄ aryl, or a C₅-C₁₄ aryloxy group;
Z represents H, C₁-C₈ alkyl, C₅-C₁₄ aryl, or a C₅-C₁₄ aryloxy group;
R represents halogen, radiohalogen, or a C₁-C₈ alkyl, C₅-C₁₄ aryl, C₁-C₈ alkylthio, C₁-C₈ alkoxy, C₂-C₆ alkenyl, C₂-C₆ alkynyl, C₃-C₁₂ cycloalkyl, or Sn(C₁-C₄ alkyl)₃ group;
R_{b} represents halogen, radiohalogen, or a C₁-C₆ alkoxy or C₁-C₈ alkanoate group, an androgen receptor binding ligand linked to the said compound via a cleavable linking moiety, or: 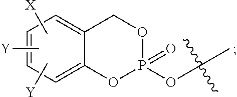
any of said alkyl, alkenyl, alkynyl, alkylthio, alkoxy and cycloalkyl group being optionally substituted by at least one halogen, OH, SH, NH₂, C₁-C₄ monoalkylamino, C₁-C₄ dialkylamino, COOH, CN, NO₂, C₁-C₄ alkyl, C₁-C₄ alkoxy or phenyl group, any of said aryl, aryloxy, and phenyl group being optionally substituted by at least one halogen, OH, SH, NH₂, C₁-C₄ monoalkylamino, C₁-C₄ dialkylamino, COOH, CN, NO₂, C₁-C₄ alkyl or C₁-C₄ alkoxy group; said radiohalogen represents ¹²³, ¹²⁴I, ¹²⁵I, ¹³¹I, ²¹¹At, ¹⁸F, ⁷⁶Br, ⁷⁷Br, or ^{80m}Br; stereoisomeric forms and pharmaceutically acceptable salts of said at least one compound;
with the proviso that at least one of the Rₐ and R_{b} substitutents represents: 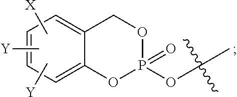
the wavy line indicating the point of attachment to the ribose moiety; and
performing imaging to diagnose the presence of said tumor.

14. The method of claim 13, wherein the androgen receptor binding ligand is selected from the group consisting of an androgen receptor agonist and an androgen receptor antagonist.

15. The method of claim 14, wherein the androgen receptor agonist is selected from the group consisting of 4-dihydrotestosterone (DHT), testosterone, mibolerone, methyltrienolone, and methyltestosterone.

16. The method of claim 14, wherein the androgen receptor antagonist is selected from the group consisting of hydroxyflutamide, flutamide, cyproterone acetate, spironolactone, ketoconazole, and finasteride.

17. The method of claim 13, wherein said imaging is selected from the group consisting of scintigraphic imaging and magnetic resonance spectroscopy.

18. The method of claim 17, wherein said scinintigraphic imaging is selected from the group consisting of positron emission tomography and single photon emission computed tomography.

19. The method of claim 13, wherein the cancer cells are selected from the group consisting of ovarian, glioma, colorectal, breast, prostate, meningioma, head and neck, or pancreatic cancer cells.

20. The method of claim 13, wherein said patient is administered a compound of the formula:

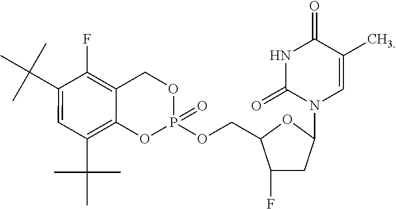 [SMILES: Cc1cn(C2CC(F)C(COP3(=O)OCc4c(F)c(C(C)(C)C)cc(C(C)(C)C)c4O3)O2)c(=O)[nH]c1=O]

21. A method for monitoring tumor activity in a subject, said method comprising administering an effective amount for diagnostic imaging of a compound of the formula: 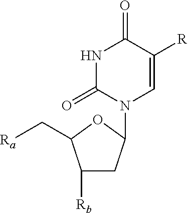
wherein Rₐ represents OH or: 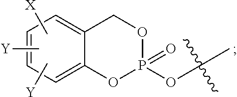
X represents H, F, Cl, or a C₁-C₈ alkyl, or C₁-C₈ alkoxy group;
Y represents H, C₁-C₈ alkyl, C₅-C₁₄ aryl, or a C₅-C₁₄ aryloxy group;
Z represents H, C₁-C₈ alkyl, C₅-C₁₄ aryl, or a C₅-C₁₄ aryloxy group;
R represents halogen, radiohalogen, or a C₁-C₈ alkyl, C₅-C₁₄ aryl, C₁-C₈ alkylthio, C₁-C₈ alkoxy, C₂-C₆ alkenyl, C₂-C₆ alkynyl, C₃-C₁₂ cycloalkyl, or Sn(C₁-C₄ alkyl)₃ group;
R_{b} represents halogen, radiohalogen, or a C₁-C₆ alkoxy or C₁-C₈ alkanoate group, an androgen receptor binding ligand linked to the said compound via a cleavable linking moiety, or: 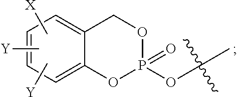
any of said alkyl, alkenyl, alkynyl, alkylthio, alkoxy and cycloalkyl group being optionally substituted by at least one halogen, OH, SH, NH₂, C₁-C₄ monoalkylamino, C₁-C₄ dialkylamino, COOH, CN, NO₂, C₁-C₄ alkyl, C₁-C₄ alkoxy or phenyl group, any of said aryl, aryloxy, and phenyl group being optionally substituted by at least one halogen, OH, SH, NH₂, C₁-C₄ monoalkylamino, C₁-C₄ dialkylamino, COOH, CN, NO₂, C₁-C₄ alkyl or C₁-C₄ alkoxy group; said radiohalogen represents ¹²³I, ¹²⁴I, ¹²⁵I, ¹³¹I, ²¹¹At, ¹⁸F, ⁷⁶Br, ⁷⁷Br, or ^{80m}Br; stereoisomeric forms and pharmaceutically acceptable salts of said at least one compound;
with the proviso that at least one of the Rₐ and R_{b} substitutents represents: 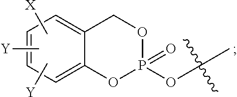
the wavy line indicating the point of attachment to the ribose moiety;
obtaining an image of said tumor to establish a baseline tumor size in said subject;
readministering said compound to said subject; and
obtaining at least one other image of said tumor producing a result which is indicative of the tumor activity in said patient.

22. The method of claim 21, wherein the androgen receptor binding ligand is selected from the group consisting of an androgen receptor agonist and an androgen receptor antagonist.

23. The method of claim 22, wherein the androgen receptor agonist is selected from the group consisting of 4-dihydrotestosterone (DHT), testosterone, mibolerone, methyltrienolone, and methyltestosterone.

24. The method of claim 22, wherein the androgen receptor antagonist is selected from the group consisting of hydroxyflutamide, flutamide, cyproterone acetate, spironolactone, ketoconazole, and finasteride.

25. The method of claim 21, wherein said subject undergoes therapy for treatment of said tumor between establishment of said baseline tumor size and obtaining said at least one other image of said tumor.

26. The method of claim 21, wherein said imaging is selected from the groupconsisting of scintigraphic imaging and magnetic resonance spectroscopy.

27. The method of claim 26, wherein said scintigraphic imaging is selected from the group consisting of positron emission tomography and single photon emission computed tomography.

28. The method of claim 25, wherein said treatment is at least one of surgical excision, radiation therapy and chemotherapy.

29.-31. (canceled)
